(19) United States
(12) Patent Application Publication
(10) Pub. No.: US 2009/0281386 A1
(43) Pub. Date: Nov. 12, 2009

(54) DEVICES, SYSTEM AND METHODS FOR MINIMALLY INVASIVE ABDOMINAL SURGICAL PROCEDURES

(76) Inventors: Pablo G. Acosta, Newark, CA (US); Dane A. Johnson, San Francisco, CA (US); Timothy A. Limon, Cupertino, CA (US); Beverly Huss, Menlo Park, CA (US); Shuji Uemura, San Francisco, CA (US); Brian K. Shiu, Sunnyvale, CA (US); Theodore M. Bender, Oakland, CA (US); Matthew B. Newell, Portola Valley, CA (US); Joshua Makower, Los Altos, CA (US); Crystine M. Lee, San Rafael, CA (US)

Correspondence Address: LAW OFFICE OF ALAN W. CANNON, 942 MESA OAK COURT, SUNNYVALE, CA, 94086, US

(21) Appl. No.: 12/474,070

(22) Filed: May 28, 2009

Related U.S. Application Data
(63) Continuation-in-part of application No. 11/716,985, filed on Mar. 10, 2007.
Application No. 11/716,985 is a continuation-in-part of application No. 11/716,986, filed on Mar. 10, 2007.
Application No. 11/716,986 is a continuation-in-part of application No. 11/407,701, filed on Apr. 19, 2006.
(60) Provisional application No. 61/130,244, filed on May 28, 2008.

(51) Int. Cl. A61B 1/01

(52) U.S. Cl. 600/114

(57) ABSTRACT

Apparatus, tools, device and methods provided for treating a patient including: making an incision or puncture though the patient's skin; establishing an initial tract through an opening formed by the incision or puncture; inserting a guide member having a flexible distal portion and a distal tip into the initial tract and extending the initial tract to form a delivery tract leading to a target location within the patient's body; wherein said distal tip and at least a portion of a remainder of said guide member are transparent and delivering an obturator and conduit assembly over said guide member to place a distal end of the conduit of said obturator and conduit assembly in a location at or near the target location, said obturator having been inserted into said conduit prior to delivering said assembly over said guide member such that a distal end of said obturator extends distally out of a distal opening of said conduit wherein said obturator comprises a central lumen adapted to closely follow said guide member while sliding thereover.

### CROSS-REFERENCE

This application is a continuation-in-part application of co-pending application Ser. No. 11/716,985, filed Mar. 10, 2007 to which application we claim priority and which application is incorporated herein, in its entirety, by reference thereto.

This application is a continuation-in-part application of co-pending application Ser. No. 11/716,986, filed Mar. 10, 2007, to which application we claim priority and which application is incorporated herein, in its entirety, by reference thereto.

This application is a continuation-in-part application of co-pending application Ser. No. 11/407,701, filed Apr. 19, 2006 to which application we claim priority and which application is incorporated herein, in its entirety, by reference thereto.

This application claims the benefit of U.S. Provisional Application No. 61/130,244, filed May 28, 2008, which application is hereby incorporated herein, in its entirety, by reference thereto.

This application also hereby incorporates herein by reference thereto, in their entireties, co-pending application Ser. No. (application Ser. No. not yet assigned, Attorney's Docket No. EXPL-008) filed on even date herewith, and titled “Minimally-Invasive Methods for Implanting Obesity Treatment Devices” and co-pending application Ser. No. (application Ser. No. not yet assigned, Attorney's Docket No. EXPL-012) filed on even date herewith, and titled “Minimally-Invasive Methods for Implanting Obesity Treatment Devices”.

### FIELD OF THE INVENTION

The present invention relates to the field of min ally invasive surgery, and more particularly to methods, devices and systems employing an endoscope for at least part of a procedure.

### BACKGROUND OF THE INVENTION

There is a current ongoing trend toward the advancement of minimally invasive surgical techniques. Such techniques not only reduce the amount of trauma to the patient but consequently reduce the amount of recovery time needed for healing, thereby reducing the lengths of hospital stays and, in some cases, even making it possible to perform procedures on an outpatient basis, such as in a physician's office.

Examples of existing procedures include laparoscopic procedures, wherein a procedure is conducted transdermally to reach an internal surgical target location. Typically this involves the formation of several (typically three or more) ports or openings through the skin and into the patient for placement of an endoscope through one opening and tools, instruments, devices through the other openings.

Other examples of existing procedures include those where an endoscope and or other instrumentation is inserted through a natural orifice, such as the mouth, anus, vagina, etc. The endoscope/instrument may be advanced along a natural pathway and then used to access the surgical site by piercing through a natural conduit forming the natural pathway. Alternatively, a procedure may be performed within the natural pathway, or on the natural conduit forming the natural pathway.

In any of these cases, the use of an endoscope may be limited when obstacles are present in a pathway leading to the surgical target location. Such obstacles may be fat or other soft tissue obstruction, tumors, or even the fact that the route from the insertion location of the endoscope/instrument to the surgical target location is very tortuous, making it difficult to establish a pathway to the surgical target location.

While the use of guidewires can improve the chance of successfully advancing an endoscope or other tool in such instances, it must first be successfully inserted and this requires external visualization guidance, such as by fluoroscopy or the like. Another current need is to reduce the use of fluoroscopy in minimally invasive procedures so as to reduce the amount of exposure of radiation to the patient and surgeon/surgical team. Also, a guidewire may not be very effective if blunt dissection is required at one or more locations along a delivery tract that is established for accessing the surgical target location.

It would therefore be desirable to provide a device or tool that is capable of establishing a tract from a location outside of a patient through an opening and to a surgical target location inside of the patient even when such tract is necessary to be curved or tortuous. It would further be desirable if such tool or device were configured to allow direct visualization while such tract is being formed.

It would still further be desirable to provide such tool or device with a steering function that can be controlled by an operator outside of the patient's body.

It would also be desirable to provide such a tool or device with a capability of changing the flexibility characteristics of at least the distal end portion of the tool or device. It would be desirable to provide such a tool or device with a capability to change at least a distal end portion thereof from being flexible to rigid and vice versa.

It would be further desirable to provide tools, instruments or devices configured to be placed along the tract, after establishment of the tract, to allow other tools and or implants to be delivered therethrough.

### SUMMARY OF THE INVENTION

The present invention provides apparatus, tools, devices and methods for treating an obese patient to facilitate weight loss.

A guide device is provided that is configured to be inserted through an opening in the skin to form a delivery tract to a target location within the body of a patient. In at least one embodiment, the guide device includes: an elongated tube, at least a portion of which is transparent a transparent distal tip closing a distal end of a main lumen of the elongated tube and having a blunt exterior surface; wherein a proximal end of the main lumen of the elongated tube is open and communicates with the main lumen, the main lumen extending through the elongated tube; and wherein a distal portion of the elongated tube is flexible.

In at least one embodiment, the distal tip comprises a lens.

In at least one embodiment, the lens comprises an exterior surface having a first curvature and an interior surface having a second curvature different from the first curvature.

In at least one embodiment the device includes a rigid tube configured to be sidably received within the elongated tube, a distal portion of the elongated tube being flexible, at least a portion of the rigid tube being slidable into the distal portion to temporarily rigidify the distal portion.

In at least one embodiment, the rigid tube comprises a central lumen configured to slidaby receive an endoscope therethrough.

In at least one embodiment, the device includes an actuator extending externally of the elongated tube, the actuator being actuatable to slide the rigid tube relative to the elongated tube.

In at least one embodiment a distal end portion of the elongated tube is pre-bent along a curvature, relative to a longitudinal axis of a straight main portion of the elongated tube, the pre-bent portion configured for steering the device.

In at least one embodiment, the guide device further includes an extension tube, a distal end portion of the extension tube being configured to be slid over a proximal end portion of the elongated tube and the actuator in the inactive configuration.

An assembly for providing access to a target location within the body of a patient is provided, including: a guide member comprising an elongated tube, at least a portion of which is transparent and at least a portion of which is rigid, or stiffened by receiving a stiffening member therein, and a transparent distal tip closing a distal end of a main lumen of the elongated tube and having a blunt exterior surface, a proximal end of the main lumen of the elongated tube being open; a conduit having a length sufficient to extend from the target location, at a distal end of the conduit through an opening in the body such that a proximal end of the conduit extends out of the body, at least a portion of the conduit being flexible, the conduit configured to allow at least one device or tool to be delivered therethrough; and an obturator having a length greater than an length of the conduit, the obturator being flexible and configured to be inserted through the conduit, a distal end portion of the obturator configured to extend distally of a distal end of the conduit and seat against the distal end, the obturator further comprising a central obturator lumen, the central obturator lumen configured to closely follow over the guide member, so that when inserted into the conduit the obturator and the conduit form an obturator and conduit assembly and when the obturator and conduit assembly is passed into the body over the guide member, the distal end portion of the obturator, together with the guide member substantially prevents bodily fluids and tissues from entering the conduit.

These and other features of the invention will become apparent to those persons skilled in the art upon reading the details of the apparatus, tools, devices and methods as more fully described below.

### BRIEF DESCRIPTION OF THE DRAWINGS

FIGS. 1A-1B illustrate an embodiment of a guide according to the present invention.

FIGS. 2A-2C illustrate an embodiment of a guide according to the present invention in which a distal end portion of a tube is flexible, while the proximal end portion of the tube is rigid.

FIG. 3A illustrates an embodiment of a guide having a single, flexible, transparent tube and an outer sleeve that is rigid.

FIG. 3B is a longitudinal-sectional view of FIG. 3A.

FIG. 3C is cross-sectional view of FIG. 3A taken along line 3C-3C.

FIGS. 4A-4K show another embodiment (and portions thereof) of a guide according to the present invention.

FIGS. 4L-4P illustrate a variation of the assembly shown and described above with regard to FIGS. 4A-4K.

FIGS. 4Q and 4R show a stylet lock installed on a guide assembly, in the locked and unlocked configurations, respectively, according to an embodiment of the present invention.

FIGS. 5A-5I show an embodiment of tip arrangement useable with any of the embodiments of guide described herein.

FIGS. 5J-5K show an alternative connection arrangement for connecting a tip to a tube.

FIGS. 6A-6E show embodiments of a tip arrangement useable with any of the embodiments of guide described herein.

FIG. 7A is an inverted, perspective view of another embodiment of tip arrangement useable with any of the embodiments of guide described herein.

FIG. 7B illustrates a view through the tip of arrangement of tip 7A, but when tip 7A is not inverted, but is top side up.

FIG. 7C is a side view of the lip shown in FIG. 7A, which is shown in the upright orientation in FIG. 7C.

FIG. 7D shows an end view of a tip having an orientation marker according to an embodiment of the present invention.

FIG. 7E illustrates how the orientation marker of FIG. 7D appears to a user in the field of view.

FIG. 7F shows an end view of a tip having an orientation marker according to another embodiment of the present invention.

FIG. 7G illustrates how the orientation marker of FIG. 7F appears to a user in the field of view.

FIG. 8A illustrates an embodiment of a guide provided with a transparent elastic, inflatable balloon (shown in a deflated configuration in FIG. 8A).

FIG. 8B shows the embodiment of FIG. 8A with the balloon in an inflated state.

FIG. 8C shows an embodiment of a guide provided with a key or guide rail structure.

FIG. 8D illustrates an embodiment of a portion of a device, tool or instrument being guided over the key or guide rail structure of the guide of FIG. 8C.

FIG. 8E illustrates an embodiment of guide with balloon, and wherein the balloon includes a “trap door” portion or thickened portion.

FIG. 8F illustrates an endoscope inserted into a balloon to perform a visualization function while the distal end portion of a tool or instrument is placed outside of the balloon between the balloon and issues inside the body of a patient.

FIGS. 9A-9F illustrate an embodiment of use of a dilator and large cannula/introducer with guide to enlarge an opening.

FIGS. 10A-10E show another embodiment of a dilator and large cannula or introducer that can be used in any of the same manners described with regard to the dilator and large cannula described with regard to FIGS. 9A-9F.

FIGS. 11A-11C show another embodiment of a dilator and large cannula introducer according to the present invention.

FIG. 11D is a side view of the handle of the dilator shown in FIG. 11B.

FIG. 11E is a proximal end view of a handle usable with the dilator of FIG. 11B, showing avariation that includes multiple endoscope ports.

FIG. 12 illustrates an embodiment of a conduit that can be inserted through a large cannula described herein, to extend distally far past the distal end of the large cannula.

FIGS. 13A-13C illustrate another embodiment of a conduit in which at least a distal end portion thereof is flexible.

FIGS. 13D and 13E are enlarged, partial views of a proximal end portion of the conduit of FIGS. 13A-13C.

FIGS. 14A-14B illustrate a plan view and a proximal end view of an embodiment of an obturator that is configured to be placed in a conduit and used to deliver the conduit through a large cannula and over a guide to deliver a distal end portion of the conduit far distally of the large cannula, according to the present invention.

FIG. 14C illustrates an alternative embodiment of an obturator in which the shaft thereof is made of corrugated tubing.

FIG. 14D illustrates an alternative embodiment of obturator according to the present invention, in which the shaft is made of rigid links.

FIG. 14E is a perspective view of one of the links of the obturator shown in FIG. 14D.

FIG. 14F is a distal end view of the link shown in FIG. 14E.

FIG. 14G is a proximal end view of the link shown in FIG. 14E.

FIG. 14H shows the obturator of FIG. 14D installed in a conduit.

FIG. 14I shows the conduit of FIG. 14H, absent the obturator.

FIG. 14J is a partial, proximal end view of the obturator shown in FIG. 14H.

FIGS. 14K-14N illustrate another alternative embodiment of an obturator according to the present invention.

FIGS. 14O-14R illustrate another alternative embodiment of an obturator according to the present invention.

FIG. 15 illustrates an embodiment of an obturator having been inserted into a conduit according to the present invention.

FIGS. 16A-16C illustrate an alternative embodiment of conduit according to the present invention.

FIGS. 17A-17D illustrate further alternative embodiments of conduit according to the present invention.

FIGS. 18A-18D illustrate alternative embodiments of conduit and obturator according to the present invention.

FIG. 19 illustrates an optional feature that may be provided with a conduit according to the present invention to resist stretching of the conduit and/or to resist axial compression of the conduit.

FIG. 20A is a partial view of an embodiment of an endoscope that may be inserted into a guide according to the present invention.

FIG. 20B shows a longitudinal sectional view of the endoscope in FIG. 20A.

FIGS. 21A-21U illustrate an example of a single access procedure and variations thereof for percutaneously implanting an extra-gastric, paragastric device according to an embodiment of the present invention.

### DETAILED DESCRIPTION OF THE INVENTION

Before the present devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and or materials in connection with which the publications are cited.

It must be noted that as used herein and in the appended claims, the singular forms “a”, “an”, and “the” include plural referents unless the context clearly dictates otherwise. Thus, for example, reference to “a tool” includes a plurality of tools and reference to “the handle” includes reference to one or more handles and equivalents thereof known to those of ordinary skill in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

A “proximal” end of an instrument is the end that is nearer the surgeon when the surgeon is using the instrument for its intended surgical application.

A “distal” end of an instrument is the end that is further from the surgeon when the surgeon is using the instrument for its intended surgical application.

### Devices, Systems and Methods

The preferred embodiments of the present invention facilitate minimally-invasive establishment of a tract from an opening in a patient that opens to the outside of the patient, to a surgical target location located internally of the patient in the abdominal cavity. Direct visualization through a preferred device is possible during the establishment of such tract.

Preferred embodiments herein further provide devices that are advanceable over a device used to establish the tract, to temporarily place a device through which an implant and/or other tools can be inserted and delivered to the surgical target location.

In preferred embodiments, a minimally-invasive procedure does not require putting the patient under general anesthesia and insufflation is not required. Preferably, only a single small opening is required for insertion of the tools/devices and optionally, an implantable device. The small opening will generally be less than about 2.5″ in diameter, or less than about 2.2″ in diameter, or less than about 2″ in diameter, or less than about 1.5″, less than about 1.25″ or less than about 1.0″ in diameter. Alternatively, more than one opening may be used for viewing through and/or inserting additional instruments. Further alternatively, minimal amounts of insufflation may be used. Also, the surgeon always has the option of using general anesthesia, though it may not required by methods described herein.

Weight loss is achieved by restriction of the stomach and filling of the space into which the stomach normally expands into in the abdominal cavity when filled with food. An implantable device expands outwardly when filled to occupy space within the abdominal cavity such that when food is ingested the stomach is restricted from being able to hold any more than a small volume of food. The implantable, outwardly expandable device is implanted outside of the stomach in the left upper quadrant of the abdominal cavity to achieve these functions. The expandable portion of the implantable device does not pierce or encircle nerve tissue or other tissue. The implantable, expandable device may be positioned with direct visualization (i.e., using an endoscope) and/or fluoroscopic visualization. No dissection, suturing, attachment or other massive manipulation or trauma into or on the stomach is required in order to implant the implantable, expandable device. By appropriate placement of the implantable, expandable device, the device can achieve restriction of the stomach. Further, the volume of the implantable, expandable device is adjustable so that the amount of restriction of the stomach can be adjusted. This can be advantageous over time, as the patient may be able to accept, or require, additional restriction of the stomach as weight loss progresses. Likewise, the loss of fat in the abdominal cavity may require the implantable, expandable device to be increased in volume to occupy additional space that is freed up by the weight loss. Both the shape of the implantable, expandable device and its fill volume, in combination, cause the desired stomach compression. Implant materials are chosen that are compatible with magnetic resonance imaging (MRI), computed tomography (CT) imaging, fluoroscopy, and X-ray imaging.

Implantation of the implantable, outwardly expandable device is carried out so as not to encircle any muscle or nerve tissue with the expandable member. Various implantable, outwardly expandable device sizes are provided, so that the present invention can treat a wide range of patients, with BMI's ranging from about 35 to about 50 and above, and including different rib cage dimensions. The present invention minimizes stress to the stomach.

For each of the embodiments of blunt-tipped guide described herein, the blunt-tipped guide either alone, or together with a stiffener such as a stylet and or endoscope, has sufficient column strength to enable the guide to be pushed through the abdominal cavity between fascia and bowel, through fat, etc.

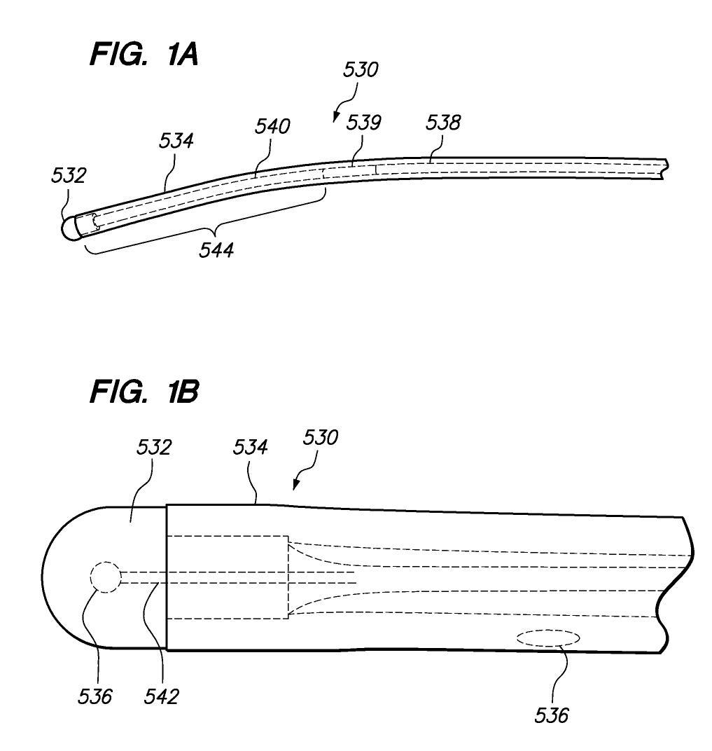

FIGS. 1A-1B illustrate an embodiment of a guide according to the present invention. Guide 530 is provided with a blunt, atraumatic distal tip 532 with bluntness provided by the curvature of the distal end of the tip 532. Guide 530 includes an elongated, flexible tube 534 that has a flexible portion at at least its distal end portion (excluding distal tip 532) when in an unreinforced configuration, as illustrated in FIG. 1A. Tube 534 may be formed of polyvinyl chloride (PVC) to ensure that the tube is transparent for maximizing visualization via an endoscope 330 inserted therein. Alternatively, polyethylene, polyurethane, PEBAX or MILIFLEX® (thermoplastic elastomer, thermoplastic olefin, Melitek, Dusseldorf, Germany) may be used. Tube 534 typically has a length of about eighteen inches to about twenty-six inches, typically about twenty inches to about twenty-four inches, although this length may vary depending upon the tract length along which guide 530 is to occupy, which will, of course vary with such factors including, but not limited to: surgical target location, location of the external opening through which guide 530 is inserted, age of the patient (e.g., child vs. adult), etc. In one particular example, tube 534 has a length of about 22.5″ and is a single flexible tube, wherein a stylet or rigid endoscope can be slid within the tube to rigidify it during use, when needed. In another embodiment a distal end portion (e.g., distal most length of about three inches to about eight inches, typically about four inches to about seven inches, in one particular embodiment about five and a half inches) may be flexible while the remaining proximal portion is stiff or relatively rigid so that it does not bend under use and therefore does not require the use of a stylet or rigid endoscope 330 to rigidify it.

One advantage of this embodiment is that a flexible endoscope 330 can be inserted into guide 530 without the need for a stylet. This arrangement can be advanced without a stylet due to the stiffness of the stiff proximal tube portion of guide 530. Flexible endoscope 330 can be advanced up into the flexible distal portion of guide 530 to provide views along a curved pathway of a tract leading to a surgical target location, for example. FIGS. 1A-1B illustrate an embodiment of guide 530 in which the entire length of tube 534 is flexible and of the same material and construction.

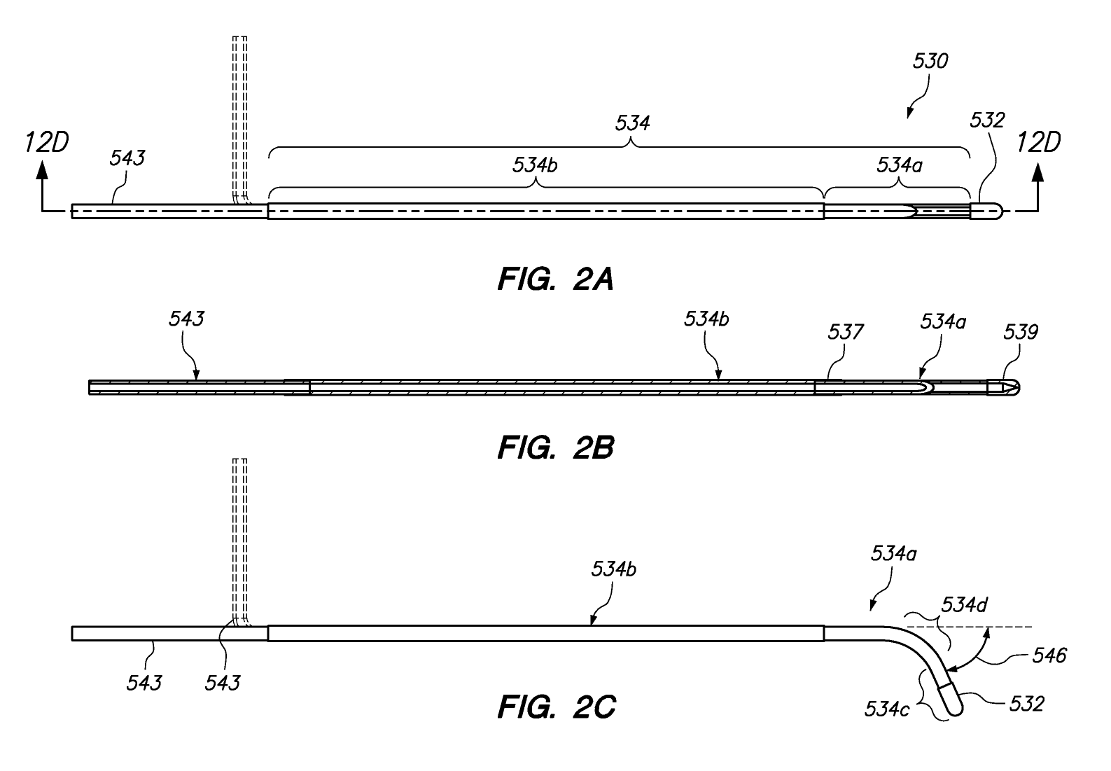

FIGS. 2A-2C illustrate an embodiment of guide 530 in which a distal end portion 534a of tube 534 is flexible, while the proximal end portion 534b of tube 534 is rigid. The tube portions 534a and 534b may be made of the same material composition, but where the hardness of the material composition used to make portion 534b is greater than the hardness of the material composition used to make portion 534a. In one particular embodiment portion 534b was made from PVC (polyvinylchloride) having a Shore hardness of 100 A, while portion 534a was made from PVC having a Shore hardness of 80 A. The clear tip 532 was also formed of PVC. In the embodiment of FIGS. 2A-2C, tip 532 does not have a lumen or opening to allow a guide wire 502 to pass through it, but is closed off, thereby preventing inflow of fluids or tissues into the tube 534. Thus, the distal end of tube 534 is closed by tip 532. Alternatively, this configuration may be provided with a lumen 536 that passes through the distal tip 532 to allow guide 530 to be passed over a guidewire 502. Likewise, embodiments of guide 530 comprising a tube 534 that is flexible over its entire length need not be provided with an opening through tip 532 or at any location of the distal end portion, but may be closed off to prevent fluid inflow, alternative to the embodiment shown in FIGS. 1A-1B. Although not shown, embodiments of guide 530 of the type shown in FIGS. 2A-2C may include one or more radiopaque markers along any locations thereof to facilitate tracking of the guide under fluoroscopy.

The longitudinal sectional view of FIG. 2B illustrates the interconnection of the tube portions 534a and 534b at joint 537. Joint 537 maybe a lap joint, a sleeve joint or other known mechanical configuration and or joined with adhesive, ultrasonic welding, heat welding, etc. Tip 532 is joined to the distal end of tube 534 at joint 539 which may be any of the same types and/or methods of joining described with regard to joint 537. Rigid portion 534b, in one embodiment, had an outside diameter of about 0.5 inches and an inside diameter (formed by the lumen passing therethrough) of about 0.225 to about 0.25 inches.

Optionally, any embodiment of guide 530 described herein may be provided with an extension tube 543 like that illustrated in FIGS. 2A-2C. Extension tube 543 may have a length of about four inches to about ten inches, typically about five to about eight inches and may be rigid or flexible. Extension tube 543 is configured to be maintained outside of the patient's body at all times, but provide an additional length for grasping by the user in instances where nearly all of the tube 534 is inserted into the body. Extension 543 further facilitates introducing a tool or implant device over the guide 530, particularly when there is not much length of the tube 534 extending out of the patient's body. Optionally, extension tube 543 may be provided to be easily removable, such as by a screw threaded joint with the proximal end of tube 534, for example, to allow installation or removal during use of the guide 530. In instances where extension tube 543 is flexible, it may be bent transversely to the longitudinal axis of the guide 530, as illustrated in phantom lines in FIGS. 2A and 2C. This may be desirable for example for use as an endoscope port, particularly when a flexible endoscope is used. In the particular example shown, extension tube 543 has a length of about six inches, is flexible, and is made of PVC having a Shore hardness of about 80 A.

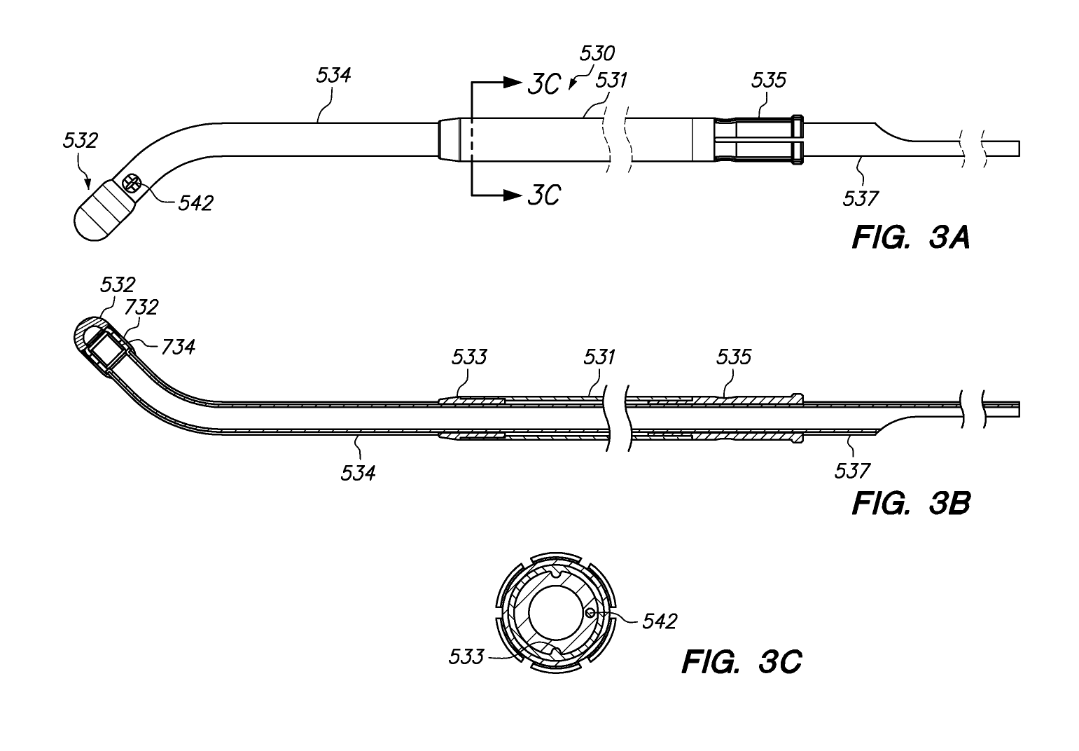

In another embodiment where tube 534 is a single, flexible, transparent tube (e.g., see embodiment of FIGS. 3A-3B), an outer sleeve 531 is provided that is rigid, thin-walled and fits closely over tube 534 while still allowing tube 534 to freely slide relative to sleeve 531. In this way, sleeve 531 can be slid over tube 534 (whether or not a flexible endoscope 330 has been inserted into the guide 530) to function like the stylet or rigid endoscope described in the embodiment above. Sleeve 531 can be translucent or opaque, but is preferably transparent, and, for example, can be made of PVC. Sleeve 531 may be keyed to tube 534 via one or more keys 533 as illustrated in the cross-sectional view of FIG. 3C and the longitudinal sectional view of FIG. 3B. A keyed collet shaft 535 may also be provided to couple and key a shaft extender 537 to the rigidizer 531.

Key 533 allows torque to be transferred to guide 530 by the user torquing on sleeve 531, which is useful for steering guide 530 as well as applying other rotational forces for repositioning and/or controlling movements of guide 530. Likewise, key 535 allows torque transfer between extender 537 and shaft 531.

The outside diameters of tube 534 and tip 532 are typically in the range of about 0.35 inches to about 0.7 inches, typically about 0.5 inches. In one example, tube 534 and tip 532 each have an outside diameter of about 0.4 inches. Tip 532 is blunt and formed of a polymer, such as PVC or acrylic polymer, to ensure that guide 530/tip 532 will not penetrate tissues such as bowel or other internal body structures not intended to be penetrated, and will not cause trauma to any of these tissues or structures. Tip 532 and/or tube 534 may optionally be provided with one or more radiopaque markers 536 at any locations) therealong, to aid fluoroscopic visualization. Rod 538 will typically be made of a material that is visualizable under fluoroscopy and thus will not require a radiopaque marker since it can be visualized without the need for one.

A stiffening rod stylet 538 is provided that is sidable through lumen 540 of tube 534 for the embodiment of FIG. 1A. Accordingly, when the distal end portion of tube 534 does not contain rod 538, it is flexible and floppy and functions similar to a guidewire, albeit with a less traumatic tip 532. However, in situations such as when there is too much resistance from fatty tissues or other tissues or obstruction to allow tube 534 to be pushed along the intended tract rod 538 (or a rigid endoscope 330 or sleeve 531) can be slidably advanced into (or over, e.g., when sleeve 531 is used in the embodiment of FIG. 3B) the distal end portion to increase the stiffness of the distal end portion. Rod 538 is continuously positionable so that the distal tip 539 thereof can be located anywhere along tube 534 with lumen 540. Likewise, sleeve 531 is continuously positionable. Accordingly, the amount of stiffness of the distal end portion of tube 534 is also continuously variably adjustable. In one embodiment, rod 538 is formed of aluminum. Alternatively, rod 538 may be formed of any other rigid, biocompatible metal, alloy, polymer and/or ceramic/composite, or the rod 538 can be a rigid endoscope, for example a glass scope with a steel sleeve for rigidity. Rod 538 can be advanced within tube 534 as described, and this runs no risk of damaging any tissues, since rod 538 is contained entirely within tube 534 and tip 532. Also, the blunt configuration of tip 532 ensures that no tissues such as bowels, diaphragm, or other soft tissues will be penetrated or traumatized even when rod 538 has been inserted all the way distally, into distal tip 532, where guide 530 is in its stiffest configuration. Further since blunt tip 532 is transparent, viewing through it via endoscope 330 is also possible. Accordingly, guide 530 also functions as a blunt introducer, and further provides visualization capabilities.

Tube 534 may optionally be provided with a lumen 542 that runs alongside the main lumen of tube 534 to facilitate delivering guide 530 over a guide wire 502 in an optional alternative procedure, or to deliver anesthetic or other fluids, as described above as well as in examples below. Lumen 542 may be provided in a separate tube, but is preferably a secondary lumen in tube 534, like that described with regard to 541 below. Alternatively, the lumen 542 can allow for an exchange with a guidewire 502. In this embodiment the guide 530 would enable placement of a guidewire 502 in a desired location by first enabling the user to place the guide 530 in the desired location. The guidewire 502 would be pre-assembled in the lumen 542, or it could be inserted into the lumen by the user. The guidewire 502 would be pushed out the front of the lumen, while the guide would be retracted from the patient. This exchange would leave the guidewire 502 at the desired location, were it would not have otherwise been able to access without the assistance of the guide 530. The guidewire 502 could then be used to guide placement of an implant.

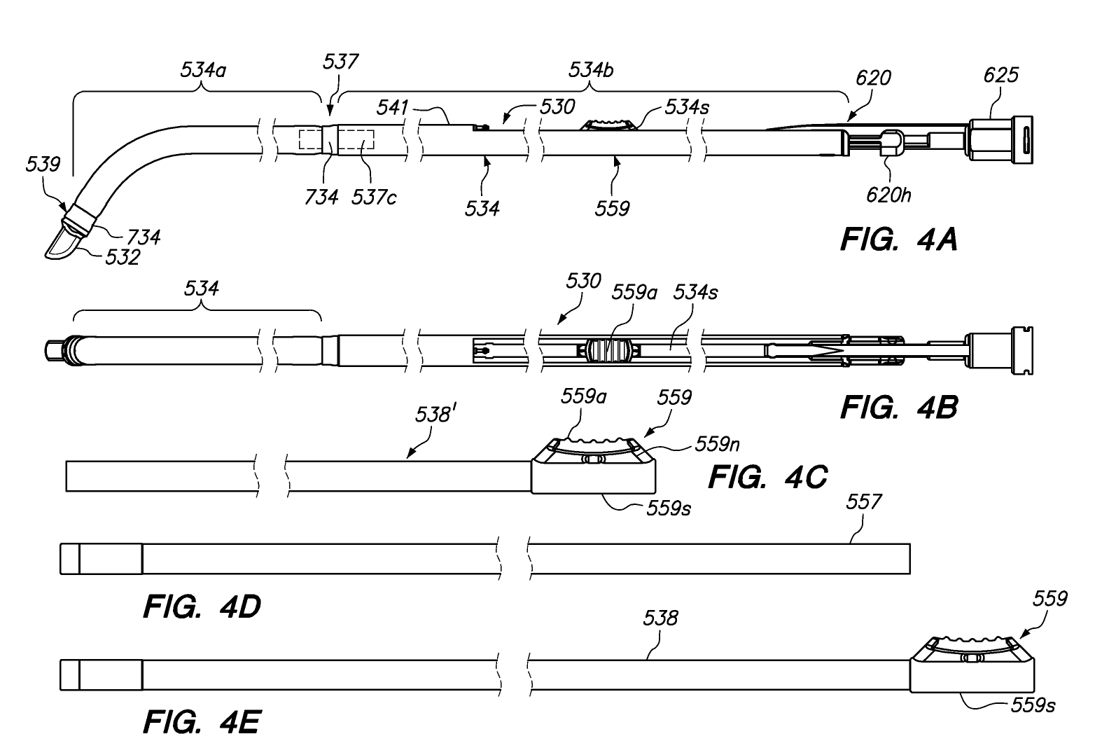

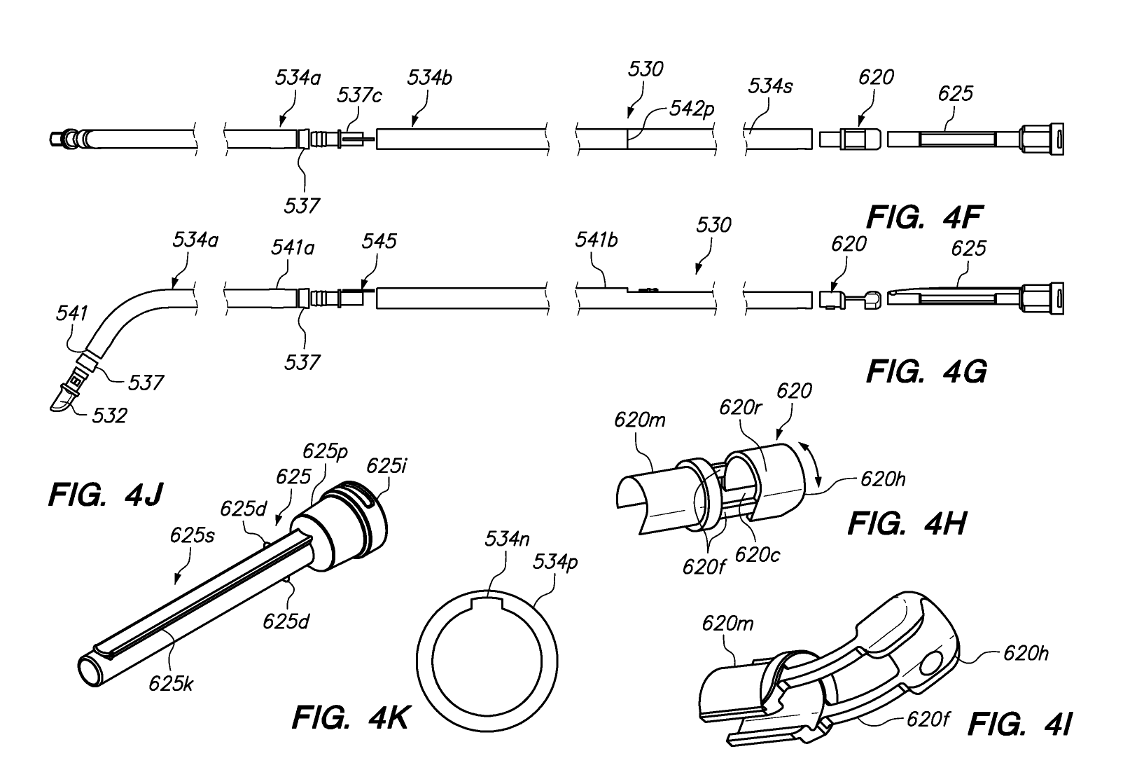

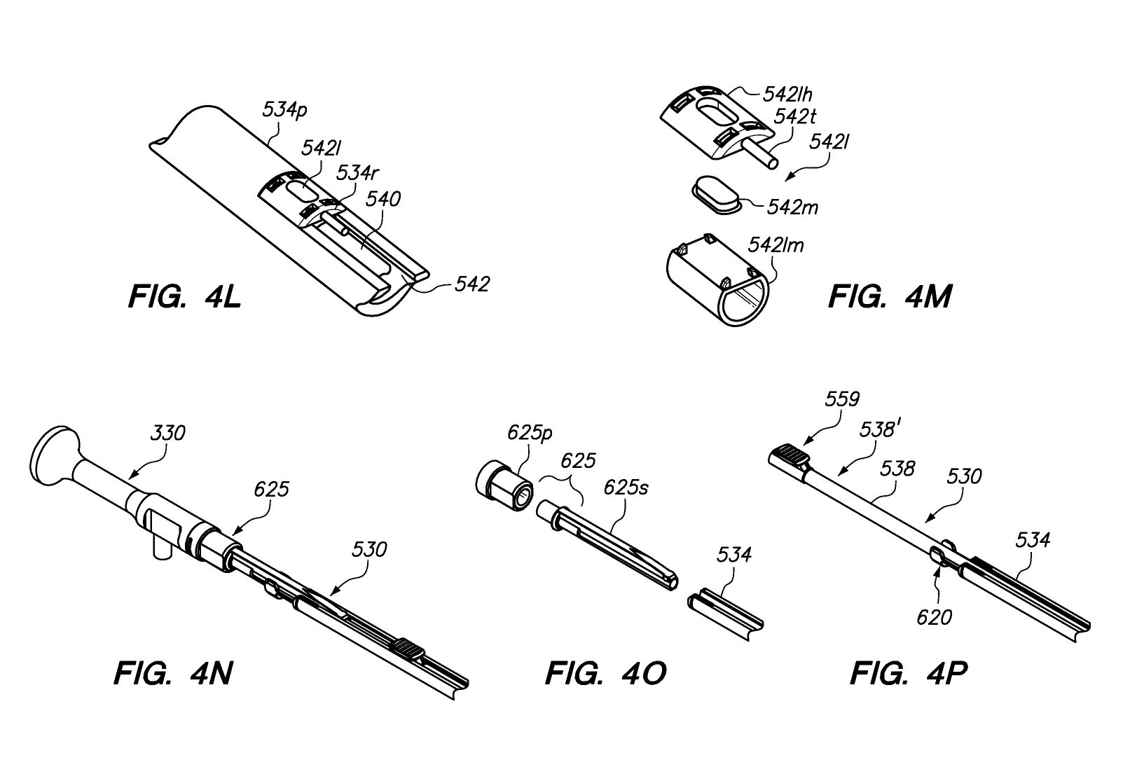

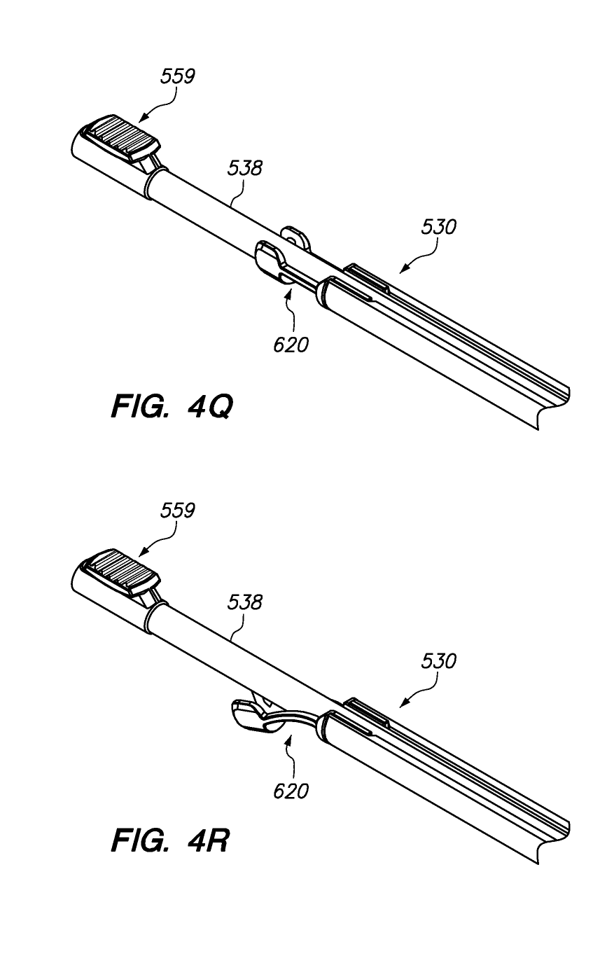

FIGS. 4A-4K show another embodiment (and portions thereof) of a guide 530 according to the present invention. FIG. 4A shows a side view of guide assembly 530 and FIG. 4B shows a view of the assembly 530 of FIG. 4A after rotating the assembly 530 ninety degrees counterclockwise about its longitudinal axis, as viewed from the proximal end of the assembly. In this embodiment the distal end portion 534a of tube 534 is flexible, while the proximal end portion 534b of tube 534 is rigid. The tube portions 534a and 534b may be made of the same material composition, like the embodiment of FIG. 2A, and may have about the same length ranges. In one particular embodiment, distal portion 534a had a length of about seven inches and proximal portion 534b had a length of about thirty-two inches, with the entire assembly 530 having an overall length of about forty-one inches. Alternatively, flexible portion 534a may be formed of a first material and rigid portion 534b may be formed of a second material. For example, flexible portion 534a may be formed from PVC having a hardness of about 78A to about 85A, and rigid portion 534b may be made of polycarbonate. The clear tip 532 may also be formed of PVC. Radiopaque marker bands 734 also function as lock collars to maintain connections between the tip 532, tube 534a and coupling 537c used in forming joint 537.

In the embodiment of FIGS. 4A-4K, tip 532 does not have a lumen or opening to allow a guide wire 502 to pass through it, nor does it have any other opening on its distal surface, but is closed off, thereby preventing inflow of fluids or tissues into the tube 534. Thus, the distal end of tube 534 is closed by tip 532. However, a secondary lumen 541 is provided externally of the main lumen of tube 534 and extends parallel thereto (or to follows the contour thereof when tube 534 is bent) The distal end of lumen 541 is open to allow delivery of medications, irrigation, suction, etc. therethrough. Note that the lumen 541 does not extend through or into tip 532. Assembly 530 may also be provided with an injection port 542p on the surface of tube 534 that is in fluid communication with lumen 541 and that is configured to allow a user to insert a blunt tip medical hypodermic needle into, to inject medication, saline, or other fluid for delivery out of the distal opening of lumen 541.

Alternatively, the embodiment of FIG. 4A may be provided with a lumen 536 that passes through the distal tip 532 to allow guide 530 to be passed over a guidewire 502, although the preferred embodiment employs a closed tip 532.

Joint 537 is formed by coupling 437c inserted into the proximal end of tube 534a and the distal end of tube 534b, see also the exploded views of FIGS. 4F and 4O which correspond to the orientations of FIGS. 4B and 4A respectively.

As already noted, one or more lock collars 734 may be employed to provide compression of the tube portion 534a or 534b against collar 537 to help maintain the joint. As also already noted, collars 537 may be radiopaque, made from stainless steel or some other rigid, biocompatible, radiopaque material. FIG. 40 illustrates a coupling 545 used to connect the proximal portion 541b of tube 541 to the distal portion 541a. Coupling 545 may be a stainless steel tube or rigid plastic tubing, for example.

Tip 532 is joined to the distal end of tube 534 at joint 539, using a lock collar 734 like that described above. Rigid portion 534b, in one embodiment had an outside diameter of about 0.5 inches and an inside diameter (formed by the lumen passing therethrough) of about 0.3 inches, flexible portion 534a had an outside diameter of about 0.438 inches and an inside diameter of about 0.318 inches, and tube 541 had an outside diameter of about 0.04 inches to about 0.05 inches.

A stiffening rod stylet assembly 538′ is provided that is slidable through lumen 540 of tube 534, see FIG. 4C. Assembly 538′ may have the same length dimension as earlier described embodiments, or may be varied according to the overall length of tube 534. In the example shown in FIG. 4C, assembly 538′ has a length of about 30.5 inches. Stylet/rod assembly 538′ includes a slide actuator 559 that includes a slider 559s connected to the proximal end of the rod/stylet 538 (see FIG. 4E) and that is dimensioned to slide within the lumen 540 of tube 534. The actuatable portion 559a of slide actuator 559 rides externally of tube 534 as stylet/rod assembly 538′ is slid relative to tube 534 and necked portion 559n has a reduced sectional dimension and rides in a slot 534s formed in tube 534 as stylet/rod assembly 538′ is slid relative to tube 534. Accordingly, a user can slide the actuator 559 by applying a thumb to the actuatable portion 559a, for example and slide the actuator 559 while holding the tube 534 to prevent axial advancement of the tube 534. When the distal end portion of tube 534 does not contain rod 538, it is flexible, except for the less traumatic tip 532. However, in situations such as when there is too much resistance from fatty tissues or other tissues or obstruction to allow tube 534 to be pushed along the intended tract, or to change the curvature of the distal end portion 534a (note that tube 534 may be formed with a preset curve to form an angle a of about 100 to about 130 degrees, typically about 110 to 120 degrees, about 115 degrees in the embodiment shown in FIG. 4A) rod 538 (or a rigid endoscope 330 or sleeve 531) can be sidably advanced into the distal end portion to increase the stiffness of the distal end portion. Accordingly, the amount of stiffness of the distal end portion of tube 534 is also continuously variably adjustable. In one embodiment rod 538 is formed of stainless steel tubing. In one particular example, rod 538 is a stainless steel hypotube having a outside diameter of about 0.219″ and an inside diameter of about 0.205 inches. Alternatively, rod 538 may be formed of any other rigid, biocompatible metal, alloy, polymer and/or ceramic/composite.

Rod 538 is preferably provided with an external jacket or coating 557 to reduce the force required to slide the assembly 538′ through the tube 534 and also allows the stylet lock (described below) to have a deformable portion to grip and lock onto. In the example of FIG. 4D, jacket 557 is made from FEP (fluorinated ethylene propylene) tubing having an outside diameter of about 0.24 inches. Rod 538 can be advanced within tube 534 as described, and this runs no risk of damaging any tissues, since rod 538 is contained entirely within tube 534 and tip 532. Also, the blunt configuration of tip 532 ensures that no tissues such as bowels, diaphragm, or other soft tissues will be penetrated or traumatized even when rod 538 has been inserted all the way distally, into distal tip 532, where guide 530 is in its stiffest configuration. Blunt tip also prevents fluids and debris from entering the lumen of tube 534, which is desirable, as fluids and/or debris could impair the functioning of the stylet making it difficult to slide. Further since blunt tip 532 is transparent, viewing through it via endoscope 330 is performed. Accordingly, guide 530 also functions as a blunt introducer, and further provides visualization capabilities.

In some circumstances it is desirable to extend the overall length of the assembly. If the user wants to maintain the position of the tip within the body and be able to pass something of significant length over the guide 530, it desirable to have the guide lengthen and securely hold position. For this reason a stylet lock 620 is provided to releasably lock the position of the stylet assembly 538′ relative to the tube 534 at any desired location that the stylet assembly 538′ is capable of sliding to. In use the stylet 538 and jacket or coating 557 are slidable through the open channel 620c provided in stylet lock 620. The isolated view of stylet lock 620 in FIG. 4H shows channel 620c clearly. The main body 620m of the stylet lock 620 is connected to head 620h via flexures 620f Flexures 620f allow head 620h to be slightly bent away from the stylet 538/coating 577 when in an unlocked configuration as shown in FIG. 41, which allows the stylet to be slid relative to tube 534. In FIG. 4A, the style assembly is show approximately midway of its stroke, making the proximal half of tube 534 rigid. The rigidized/stiffened portion becomes longer as the stylet lock is spaced further away from the stylet assembly. Corresponding to FIGS. 4H-4I, FIGS. 4Q and 4R show the stylet lock installed on the guide assembly, in the locked and unlocked configurations, respectively. When it is desired to lock the stylet 538 to prevent its axial movement relative to tube 534, the head 620h is pressed to rotate it back into alignment with the main body 620m causing rib, tooth, or other engagement member 620r to engage against coating 557 and/or stylet 538, thereby forming a friction lock. When a coating such as jacket 577 is present engagement member presses or “bites” into the jacket 577, temporarily deforming it and enhancing the braking action. The endoscope is removed when this locking engagement is carried out.

FIG. 4J is an enlarged, isolated view of an endoscope lock 625 that may be provided with guide assembly 530. Endoscope lock 625 includes an enlarged proximal end portion 625p and an elongated shaft portion 625s extending distally from the proximal end portion 625p. The shaft portion 625s may be keyed 625k to provide an interlocking fit with a notch 534n formed in a proximal end portion of proximal tube portion 524p, see FIG. 4K. The opening 625i of the proximal portion 625p is dimensioned to form a friction fit with a proximal end portion of endoscope 330. The endoscope is affixed with two radial protrusions (or bayonets) which interlock into the grooves shown in 625i. The width of the grooves narrow as the endoscope is rotated, thus locking it in place. This frictional lock combined with the lock provided between key 625k and notch 534n prevents endoscope 330 from rotating relative to tube 534 once it has been inserted therein and locked by the scope lock 625. Accordingly, the field of view provided by the scope 330 maintains a constant orientation attitude relative to the orientation of tube 534 over the entire course of use, even when the stylet is slid back and forth. The scope lock and endoscope are removed from the guide when it is extended for purposes of passing elongated device (e.g., the conduit 600 and obturator 630 over the guide 530).

FIGS. 4L-4P illustrate a variation of the assembly shown and described above with regard to FIGS. 4A-4K. hi FIG. 4L a septum 542L is provided in the wall of tube 534p, alternative to the port 542p shown in FIG. 4F. Note that lumen 542 is alternatively configured between the external and internal walls of tube 534, rather than as an external tube as described above with regard to FIGS. 4A-4K. However, this configuration can also be provided alternatively with a port 542p. FIG. 4M shows an exploded view of septum 542L showing a main housing 542LM, a membrane 542M (e.g., silicone, or the like) and a secondary housing portion 542LH that includes a tube 542T that connects with lumen 542 to configure septum 542L in fluid communication therewith. Septum 542L is received in a recess 534r in tube 534. It is adhesively bonded in place and is low profile, to fit within the wall thickness of the rigid main outer tube.

FIG. 4N illustrates a partial (proximal end portion) view of the guide assembly 530 with an endoscope 330 having been inserted therein. Scope lock 625 includes two parts that snap together (see FIG. 40) in this embodiment and allow rotation of the endoscope 330 relative thereto, but prevent rotation of the lock 625 relative to tube 534 in the manner described above. Part 625p rotates freely with respect to 625k (see FIG. 4J), while portion 625k is friction fit into the proximal part of the slot in guide 530. The slot acts like a spring, clamping shut on the raised portion of 625k and providing stiff resistance to axial movement of 625k relative to 530. Axial motion if prevented by the frictional force between the slot and the raised portion.

The length of assembly 530 may be extended when needed, such as for guiding a conduit 600 and obturator 630 thereover, or in other situations where an extended length is desirable. FIG. 4P illustrates that lengthening may be accomplished by removing the endoscope 330 from assembly 530 and retracting the stylet assembly 538′ so that a portion of the stylet 538 extends proximally of the proximal end of tube 534. When stylet assembly 538′ has been retracted sufficiently to meet the needs of the user, the stylet lock 620 can then be locked down against the stylet 538 and or coating or jacket 557.

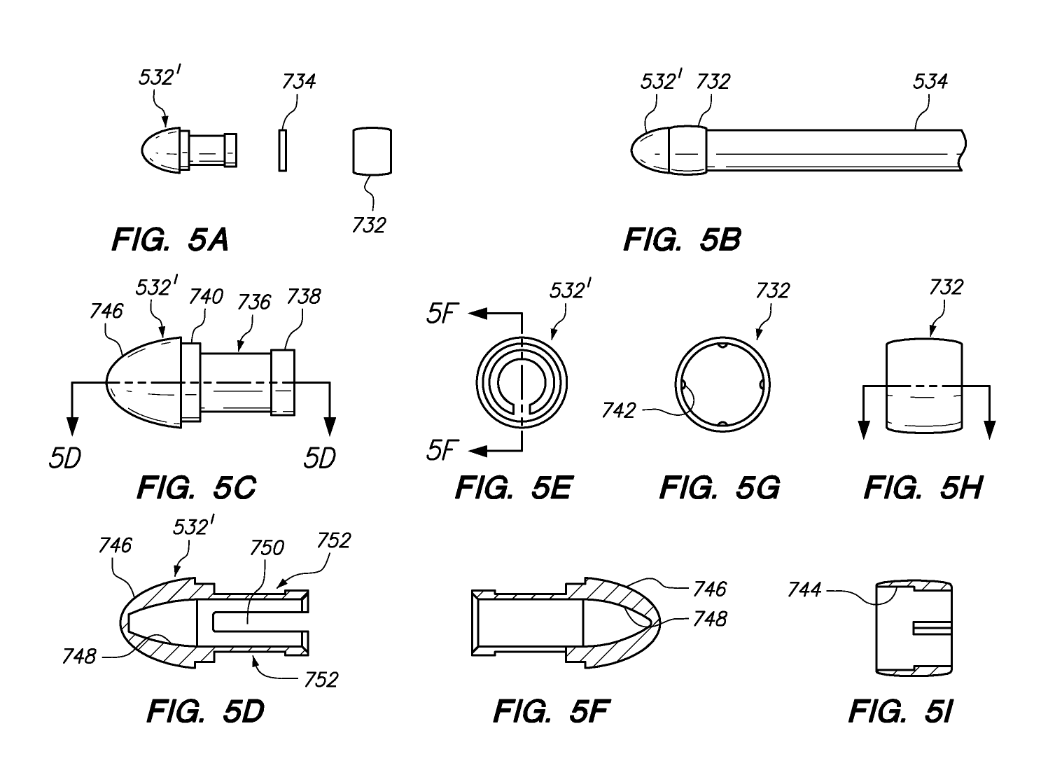

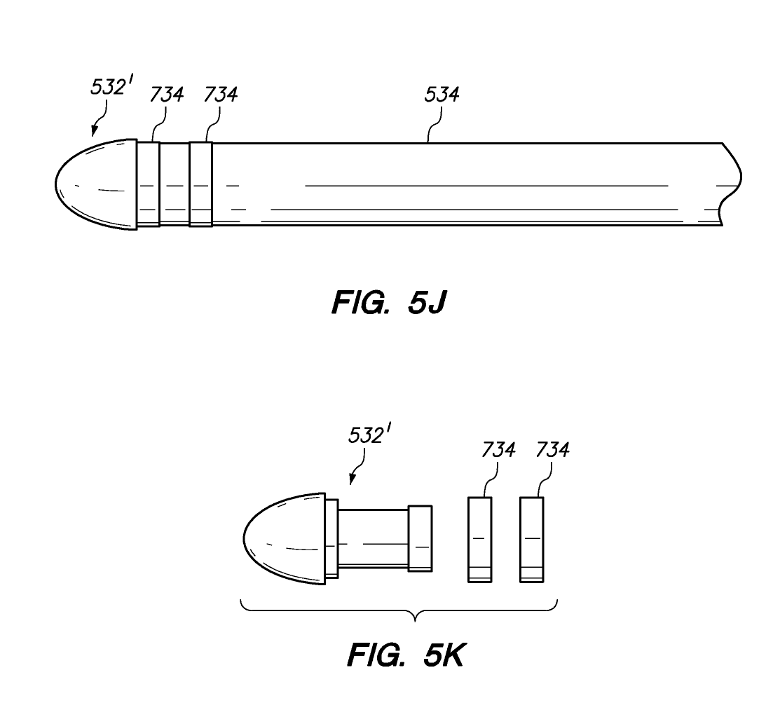

FIGS. 5A-5I show an embodiment of a tip useable with any of the embodiments of guide 530 described herein. Tip 532′ is attached to tube 534 via a compression fit using band 732. Band 732 may be radiopaque to also function as a radiopaque marker 536. Optionally, adhesive may be used to enhance the connection of tip 532′ and/or to seal the connection. Tip 532′ is provided with a blunt exterior curvature 746 that is symmetrical about 360 degrees, i.e., same curvature from the distal tip to the proximal end of the curve, regardless of the location about the circumference of the tip, as the line of curvature extends in the direction of the longitudinal axis of the tip 532′. The exterior curvature 746 is a curved, generally conical shape. Tip 532′ includes a recessed segment 736 proximal of the curved portion 746. Recessed segment 736 is bordered by shoulders 738 and 740, each of which has an outside diameter greater than the outside diameter of recessed segment 736. This arrangement is configured to receive band 732 over the location of recessed segment 736, where shoulders 738, 740 prevent axial migration of the band 732. Of course, tube 534 is positioned between band 732 and segment 736, and over shoulders 738. Band 732 and section 736 thereby maintain compressive forces against tube 534 to keep it stationary relative to tip 532′.

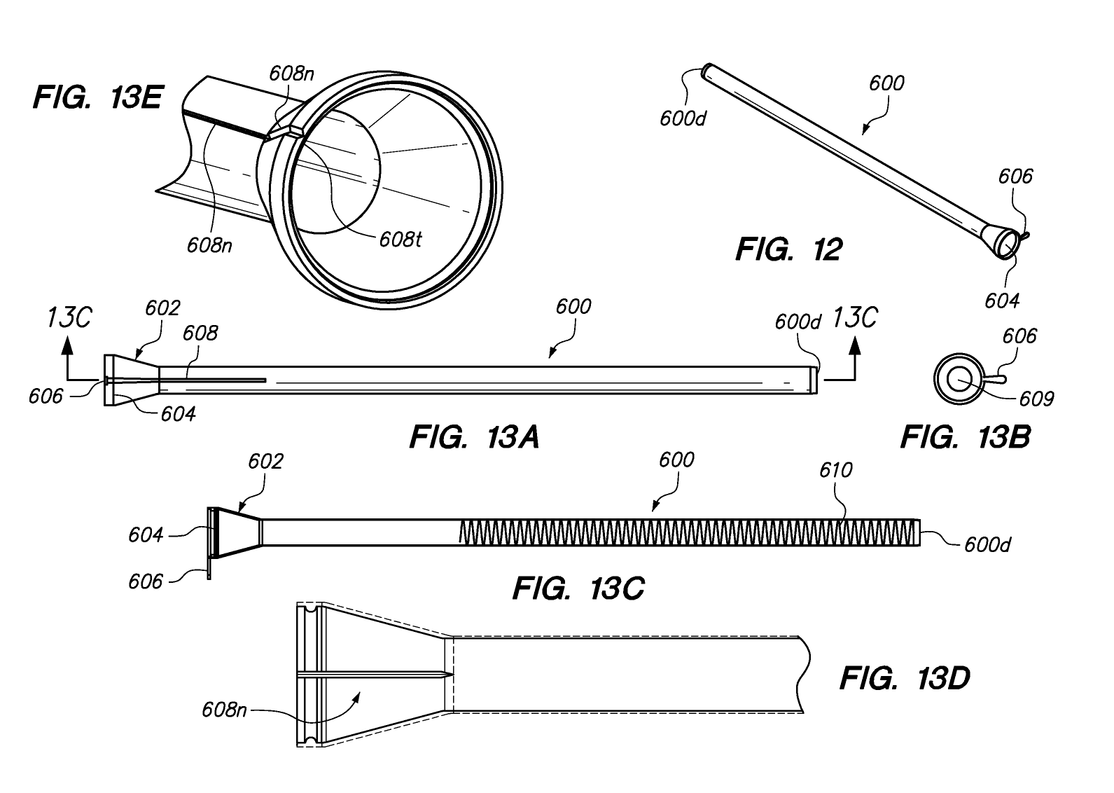

Additionally, a secondary retainer ring 734 may be provided to slide over tube 534 and reside over a portion of segment 736. This secondary retainer ring may also function as a radiopaque marker, and may thus be made of stainless steel or may be a polymer having embedded barium, for example. When secondary retainer ring 734 is used, band 732 is recessed 744 along its inside diameter at a distal portion thereof (see FIGS. 12V and 12W) to receive secondary retainer ring 734 and maintain contact therewith, as ring 734 contacts tube 534 to hold it in compression against segment 736. Likewise, the proximal portion of band 732, which is not internally recessed and therefore has a smaller inside diameter than portion 744, acts as a stop or shoulder against ring 734 and also maintains contact against tube 534, holding it in compressive contact against segment 736. The proximal portion of band 732 may further be provided with protrusions 742 (See FIG. 5S) which extend radically inwardly and further enhance the friction between band 732 and tube 534.

The proximal end portion of tip 532′ optionally includes a slot 750 that separates at least shoulder 738 and segment 736 into at least two portions. This allows the portions separated by slot 750 to be flexed toward one another, thereby temporarily reducing the outside diameters of these portions. This facilitates the ease with which the proximal end portion of tip 532′ can be slid into the lumen of tube 534 at the distal end thereof When tube 534 abuts shoulder 740 (or overlies the same, alternatively) release of compression on the portions of the proximal end portion of tip 532′ allow them to resiliently spring back to the configuration shown in FIGS. 5C and 5D. Sliding of band 732 (and optionally ring 734) over the tubing to reside in their respective locations over segment 736, completes the assembly of guide 530, as shown in FIG. 5B.

To improve the optics of the tip 532′, a secondary curvature 748 is provided on the internal surface of the tip. The secondary curvature 748 does not match the curvature 746 on the external surface of tip 532′. In one preferred embodiment, surface 748 is formed to be “duckbill-shaped”. That is, the curvature in one plane (see FIG. 5F, taken in the plane C-C of FIG. 5E, which is a proximal end view of tip 532′) is bullet-shaped, which is similar to the curved conical shape of surface 746, but with a sharper angle of curvature, and where the distal tip of the curve is not atraumatic, but much sharper than the external distal tip. In a plane perpendicular to the aforementioned plane, the surface 746 does not come to a point at the internal distal tip, or even an atraumatic soft curve, but rather is flat, e.g., a straight line generally perpendicular to the longitudinal axis, see the sectional view of FIG. 5D taken along line A-A of the side view of tip 532′ in FIG. 5C. This arrangement reduces artifacts, such as the “halo effect” and other reflections that would otherwise be observed through the tip by an endoscope inserted therein.

FIGS. 5J-5K show an alternative connection arrangement for connecting a tip 532, 532′ to a tube 534. In this example, two rings 734 are applied directly over tube 534 to compress it against segment 736. Thus, band 732 is not employed in this arrangement. As in the previous arrangement adhesive may be optionally be used to enhance the connection of tip 532′, 532 to tube 534 and/or to seal the connection.

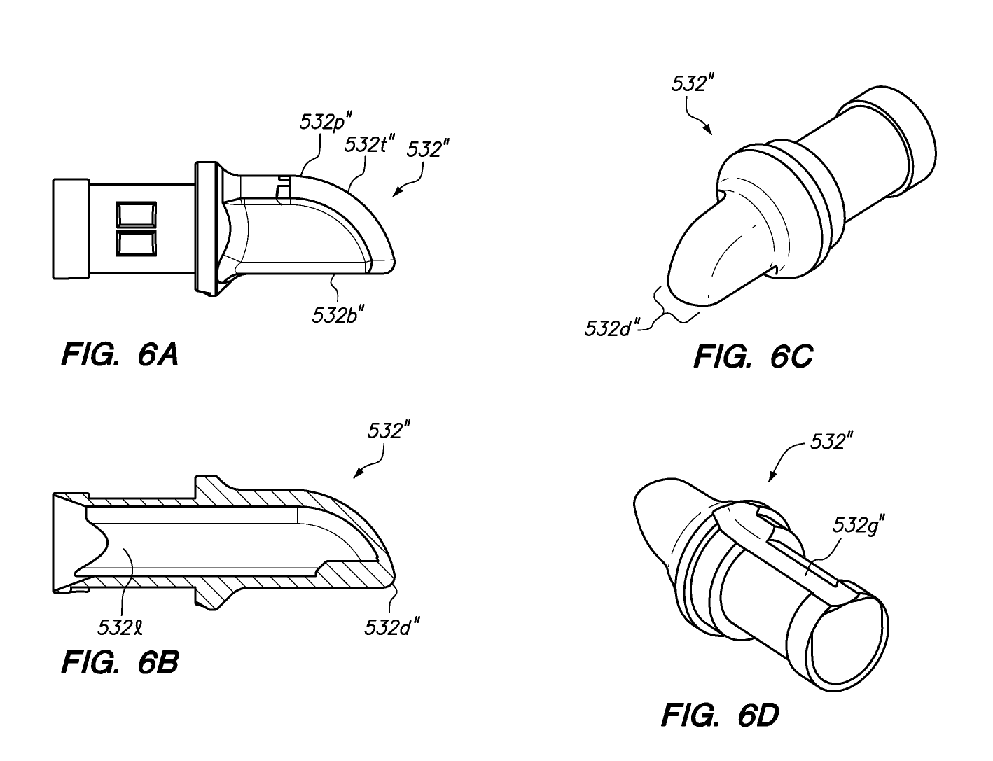

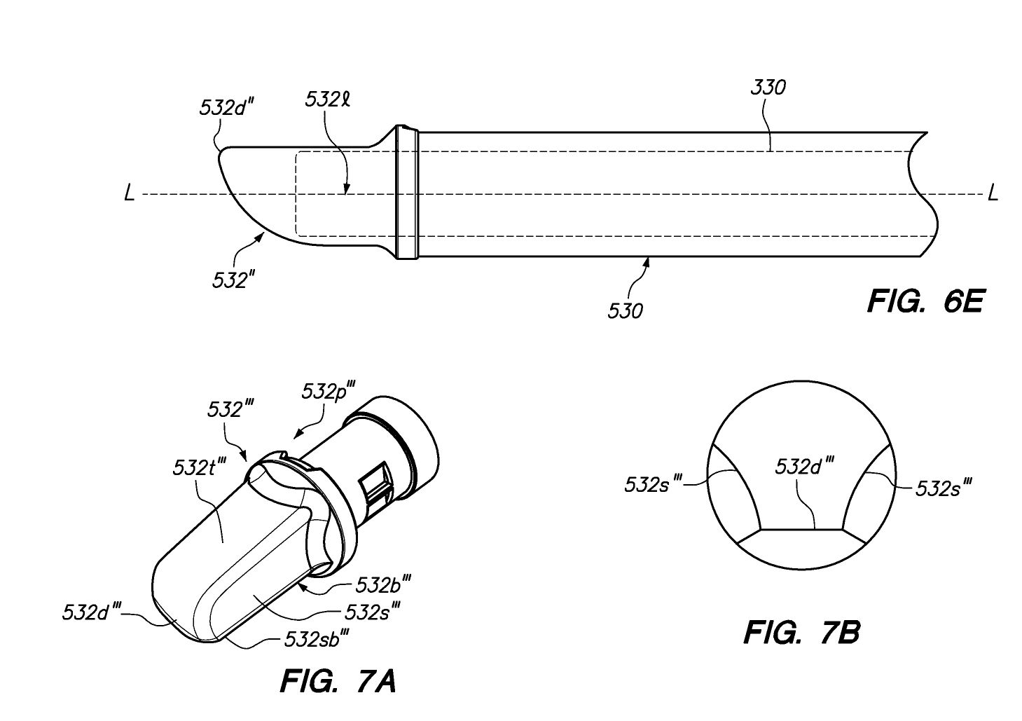

FIGS. 6A-6B show an embodiment of tip arrangement useable with any of the embodiments of guide 530 described herein. Tip 532″ may be attached to tube 530 in any of the same manners described above with regard to tip 532′. Tip 532″ may be made of any of the same transparent materials described above with regard to previously described tips 532 and 532′. Tip 532″ however, does not have a conical exterior shape, unlike the shapes of lips 532 and 532′. Rather, the outer surface of the bottom portion of tip 532″ has a tapering curvature that tapers from the circular cross-section of the proximal portion 532p″ to a blunt curved transversely extending segment 532d″ (see FIG. 6C) at the distal end of the tip, where the outer surface of the bottom portion 532t″ joins the outer surface of the top exterior portion 532b″. The outer surface of the top portion 532b″ is substantially flat (substantially planar). The curved transversely extending segment 532d″ is formed to one side of the central longitudinal axis L-L of the lumen formed in tip 532″ and therefore also extends transversely above the central axis of the lumen of the tube 534 to which it is attached, and also therefore extends transversely and above the central axis of an endoscope 330 inserted in a tube 534 to which tip 532″ is attached.

Unlike the embodiment of FIGS. 5A-5I, the inner surfaces of the tip 532″ do not have a different curvature than the outer surfaces, but generally follow the same contours. Thus, the thickness of the tip walls is substantially constant thereover, as the upper inner surface is substantially flat or planar and the inner lower surface has a curvature that substantially corresponds to the curvature of the outer lower surface. The sides of tip 532″ in this embodiment are generally vertical, with the inner surfaces having substantially the same generally vertical orientation as the outer surfaces. Because of the asymmetric configuration of the lower portion 532t″ relative to the upper portion 532b″, reflections and artifacts are greatly reduced. Also, because the curved transversely extending segment 532d″ is below the central longitudinal axis (viewing axis) of an endoscope 330 inserted into guide 530 (and optionally into tip 532″), and distortion caused by 532d″ is below the main field of view of the endoscope 330 and establishes a horizon reference line therefore. Viewing can also be accomplished below this horizon line, through upper portion 532b″.

Optionally, tip 532″ (or any of the other tips described herein) may be provided with a recess or groove 532g″ (see FIG. 6D) that is aligned with the longitudinal axis of the tip and is recessed into the external surface thereof Groove 532″ may function for alignment with a secondary lumen 542, which may be formed within the main wall of the tube 534 for example, or by an additional small tube running externally of the tube 534, and to facilitate delivery of a fluid through the secondary lumen and out of the device 530,532″. However, tip 532″ does not have an opening joining the inside of the tip to the outside of the tip once the proximal end of the lumen 5321 is closed off by mounting tube 530 thereover (see FIG. 6E).

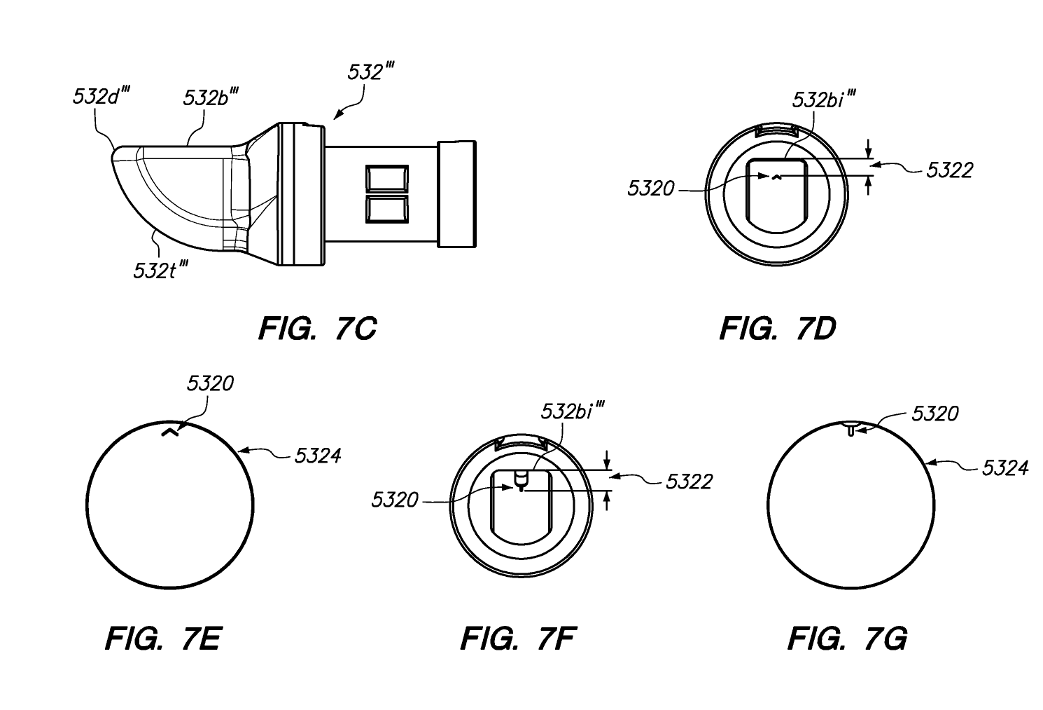

FIGS. 7A-7B show an embodiment of tip arrangement useable with any of the embodiments of guide 530 described herein. FIG. 7A shows the tip 532′″ in an inverted, perspective view to better show the details of the bottom portion 532t′″. FIG. 7B shows the view that a user would have by looking out through the distal end of tip 532′″ when the tip 532′″ is not inverted (i.e., after inverting the orientation shown in FIG. 7A). Tip 532′″ may be attached to tube 530 in any of the same manners described above with regard to tip 532′. Tip 532′″ may be made of any of the same transparent materials described above with regard to previously described tips 532, 532′ and 532″. Tip 532′″, like tip 532″ does not have a conical exterior shape, unlike the shapes of tips 532 and 532′. Rather, the outer surface of the bottom portion of tip 532′″ has a tapering curvature that tapers from the circular cross-section of the proximal portion 532p′″ to a blunt curved transversely extending segment 532d′″ at the distal end of the tip, where the outer surface of the bottom portion 532t′″ joins the outer surface of the top exterior portion 532b′″. The outer surface of the top portion 532b″ is substantially flat (substantially planar). Additionally, in this embodiment side portions 532s′″ are substantially flat. Accordingly, blunt, straight, axially extending segments 532sb′″ formed at the junctions of the side portions 532s′″ and the top portion 532b′″ extend distally from the ends of the curved transversely extending segment 532d′″ from the locations where the segments meet. The curved transversely extending segment 532d′″ and segments 532sb′″ are formed above the level of the central longitudinal axis of the lumen 5321 formed in tip 532′″. These segments are visible in the viewing field of an endoscope 330 inserted into a guide 530 fitted with tip 532′″ in a manner as illustrated in FIG. 7B. Thus, segments 532d′″ and 532sb′″ allow the user to easily identify the orientation of the tip 532′″ even when tip is inserted within the body, by viewing through endoscope 330.

Like the embodiment of FIGS. 6A-6B, the inner surfaces of the tip 532′″ do not have a substantially different curvature than the outer surfaces, but generally follow the same contours. Thus, the thicknesses of the tip walls are substantially constant thereover, as the upper inner surface is substantially flat or planar and the inner lower surface has a curvature that substantially corresponds to the curvature of the outer lower surface. The sides of tip 532′″ in this embodiment are also substantially flat, with the inner surfaces being substantially flat and thus having substantially the same conformation as the outer surfaces to maintain the wall thicknesses substantially constant. Because of the asymmetric configuration of the lower portion 532t′″ relative to the upper portion 532b′″ and sides 532s′″, reflections and artifacts are greatly reduced. Also, because the curved transversely extending segment 532d′″ and segments 532sb′″ are above the central longitudinal axis (viewing axis) of an endoscope 330 inserted into guide 530 (and optionally into tip 532′″), and distortion caused by 532d′″ and segments 532sb′″ is above the main field of view of the endoscope 330, this establishes a horizon reference line therefore. Viewing can also be accomplished above this horizon line, through top portion 532b′″.

Optionally, tip 532′″ (or any of the other tips described herein) may be provided with a recess or groove 532g″ that is aligned with the longitudinal axis of the tip and is recessed into the external surface thereof Groove 532″ may function for alignment with a secondary lumen 542, which may be formed within the main wall of the tube 534 for example, and to facilitate delivery of a fluid through the secondary lumen and out of the device 530,532″. However, tip 532′″ does not have an opening joining the inside of the tip to the outside of the tip once the proximal end of the lumen 5321 is closed off by mounting tube 530 thereover.

FIG. 7C is a side view of lip 532′″ in the upright orientation. Tip 532′″ may be provided with a marker 5320 located on the inside surface of the tip lens that is located in font and along the curvature of the lens near the top 532b′″ flat portion separated by a distance 5322 from the inner surface 532bi′″ of the top of the lens 532′″, as shown in the end view of FIG. 7D. In one embodiment distance 5322 is about 0.056″ although this distance may vary. One example of an orientation marker 5320 is a chevron-shaped orientation marker 5320 as shown in FIG. 7E which represent how the chevron 5320 would appear to a user during use. In one embodiment the line segments of the chevron 5320 are about 0.002″ to about 0.005″ wide, about 0.015″ in length and about form an angle between them of about eighty degrees and the chevron can be raised or lowered from the inner surface of the lens by a height or depth of about 0.005″, although any and all of these specifications may vary. Preferably, the chevron 5320 points to the top of the lens 532′″. When viewed by a user, the chevron 5320 appears near the top edge of the field of view 5324 of the tip 532′″.

In another embodiment, the orientation marker 5320 is in the form of a vertical line as shown in the end view of FIG. 7F. Vertical line 5320 is located as described above with regard to chevron 5320. In on embodiment the end of the vertical line 5320 furthest away from the top inner surface 532bi′″ was about 0.056″ from the inner surface 532bi′″ and line 5320 was about 0.015″ in length, with the other end connecting to the inner surface 532bi′″, the line width was about 0.005″ and was raised about 0.005′ above the inner surface of the tip lens.

However, any and all of the foregoing dimensions may vary. FIG. 7O illustrates the appearance of the line 5320 when viewed by a user. Line 5320 appears near the top edge of the field of view 5324 of the tip 532′″.

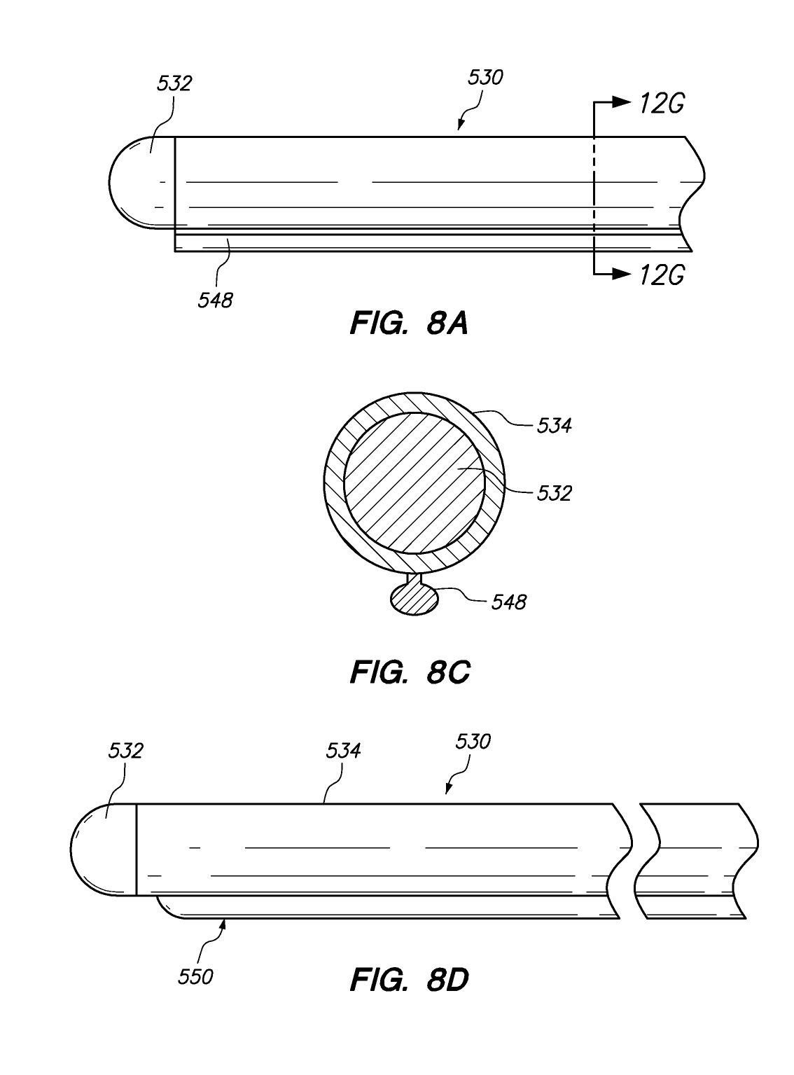

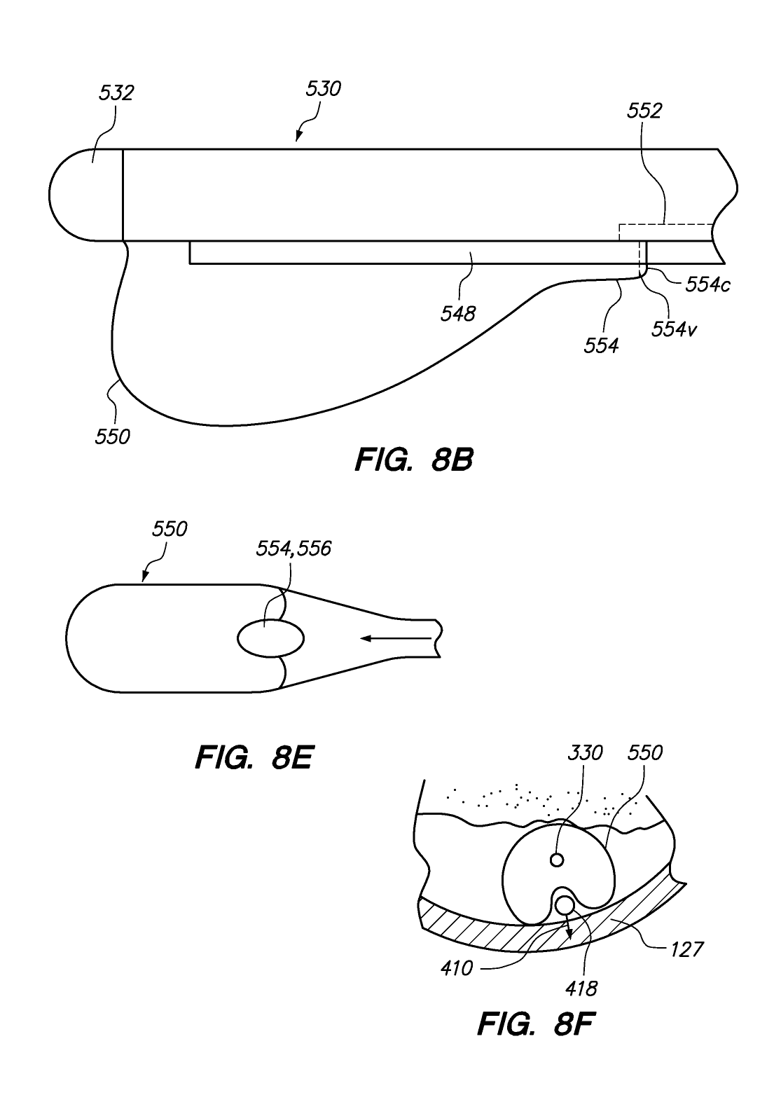

FIG. 8A illustrates an embodiment of a guide 530 provided with a transparent elastic, inflatable balloon 550 (shown in a deflated configuration in FIG. 8A). FIG. 8B shows the embodiment of FIG. 8A with balloon 550 in an inflated state. A lumen 552 extending either within tube 534, or externally alongside tube 534 allows pressurized fluid to be inputted from a location outside of the patient 1 to inflate balloon 550. An opening 554 is provided in the proximal end of the inflated balloon, to allow a tool to be inserted therein. Extending proximally from opening 554 is a conduit 554c with a valve 554v that seals around the tool after it has been inserted, in order to create a seal for inflating the balloon 550. The tool received in the balloon 550 may be configured to slidably receive an endoscope 330 therein or may be configured with an integral endoscope 330.

Guide 530 may be additionally or alternatively provided with a guide structure 558 (FIG. 8C) such as a rail, key structure or other structure (see FIGS. 8B-8D) and tools, instruments and/or devices can be provided with a feature 549 having a slotted lumen, slotted rings, or other mating component 548 to be guided along structure 558 (as illustrated in phantom lines in FIG. 8D) to provide guidance of the tool, instrument or device as it is slid thereover.

FIG. 8E illustrates an embodiment of guide 530 with balloon 550 inflated, and wherein balloon 550 includes a “trap door” portion 554 or thickened portion 556 that allows drivers of a tool or other actuation member of a tool or instrument, having been inserted within balloon 550, to be driven out of the balloon 550, where, upon retraction of drivers or other actuators back within balloon 550, the majority of the inflation fluid used to inflate balloon 550 is retained in balloon 550, either by the trap door 554 closing back up (after having been opened by the driving forces of the driver or other actuator) or the thickened portion 556 self sealing after retraction of removal of the driver(s)/actuator(s).

Further alternatively, an endoscope 330 that is independent of a tool or instrument may be inserted into balloon 550 to perform a visualization function while the distal end portion 418 of the tool or instrument is placed outside of balloon 550 between balloon 550 and the tissues 127 inside the body of the patient as illustrated in FIG. 8F. Alternatively, the tool or instrument may be configured to sidably receive endoscope 330 and may be configured so that the portion of the tool or instrument that receives endoscope 330 can be inserted into (or abutted against) balloon 550, while distal end portion 418 is positioned externally of the inflated balloon 550.

Once a tract has been established from an opening to the outside of the patient (such as by using guide 530 and optionally other instruments, more detailed examples of which are described below) toward a surgical target location within the patient, it may be desirable with some procedures to enlarge the opening to facilitate passage of one or more tools or instruments, and, optionally, one or more implantable devices along the tract.

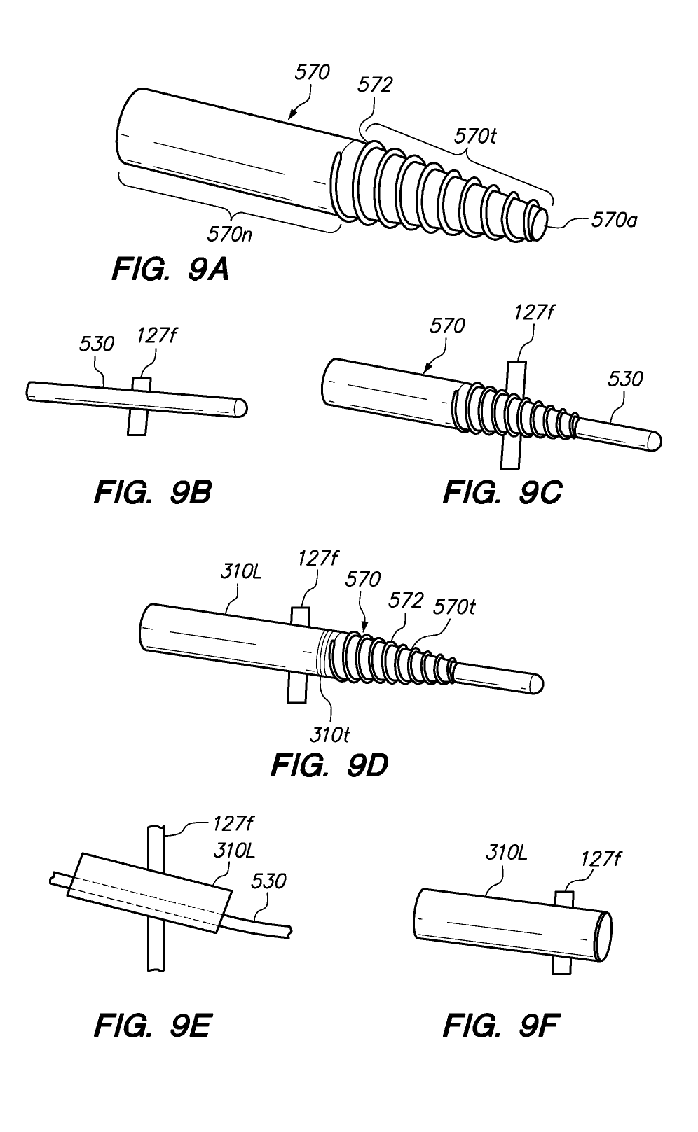

FIG. 9A-9F illustrate one embodiment of use of dilator 570 and large cannula 310L with guide 530 to enlarge an opening. In this embodiment, an opening through the fascia 127 leading into the abdominal cavity is enlarged. However, these techniques are not limited to enlarging an opening into the abdominal cavity, as they can also be used to enlarge an opening into the thoracic cavity, or to enlarge another opening leading into the patient.

FIG. 9A illustrates a dilator 570 that may be used to perform the dilation of the opening through the fascia 127f and or abdominal muscle 127. Dilator 570 is tapered, with a large thread form 572 along the tapered portion 570t and transitioning to the non-tapered portion 570n. In at least one embodiment the thread form 572 is about 1.5 threads per inch, has a pitch of about 2.67 and wherein the tapered portion has a taper of about eight degrees. In another embodiment, the thread form 572 is about 2.67 threads per inch, has a pitch of about 0.375 and the tapered portion has a taper of about eight degrees. Each of these specification may vary, but the threadform should remain large (e.g. about 1.1 to about 3.3 threads per inch) and the threads should extend sufficiently from the surface of the taper, e.g., about 0.065″ to about 0.125″, typically about 0.080″, but be blunt (rounded) so as to grab the tissues to drive the dilator into the abdominal cavity as the dilator 570 is rotated, without cutting the tissues that the threadform 572 contacts. Dilator 70 has a central annulus or lumen 570a extending therethrough which has a diameter slightly larger than the outside diameter of guide 530. Accordingly, annulus 570a may have a diameter of about 0.5″ or slightly larger. In one particular embodiment dilator 570 has an inside diameter of about 0.505″ formed by annulus or lumen 570a, and an outside diameter of the non-tapered portion is about 0.995″. In another embodiment, the outside diameter of the non-tapered portion is about 1.588″ and the inside diameter is about 0.505″. The distal end of dilator 570, where the tapered portion begins has an outside diameter of slightly greater than the annulus diameter, e.g., about 0.6″ to about 0.7″ and tapers to the cross-sectional dimension of the non-tapered section 570n, which may, for example, have an outside diameter of about 1.0 inches to about 1.7 inches. In another example, the outside diameter of the non-tapered portion 570n was about 1.2 inches. The profile of the threadform 572 can be radiused so that there are no sharp edges on the threadform 572, thereby greatly reducing the risk of trauma. Dilator 570 (including threadform 572) may be made of a relatively rigid, but lubricious polymer, such as DELRIN® (acetal copolymer) or other acetal copolymer, or other suitable biocompatible polymer, such as an injection moldable polycarbonate with or without a radiopaque filler or marker band.

FIGS. 9B-9E schematically illustrate use of dilator 570 to increase the size of the opening in the fascia 127f and/or abdominal muscle or other tissue so as to make it easier to insert an implantable device and/or tool therethrough. FIG. 9B illustrates guide 530 positioned through the fascia/abdominal muscle 127f after establishing a tract therethrough. Although not shown in the schematic illustration of FIG. 9B for reasons of simplicity of illustration and clarity, at least the portion of guide 530 passing through the opening 127f and proximally thereof at least until exiting the patient are rigid, or made at least temporarily rigid by any of the techniques described herein, so as to maintain the orientation of the guide 530 while also providing a low profile arrangement that allows the dilator to be easily passed over the proximal end of guide 530.

Dilator 570 is then slid over the proximal end of guide 530, distal end first and advanced into the opening in the patient. Dilator 570, upon reaching the fascia 127 for even prior thereto, can be rotated (clockwise if threadform 572 is arranged in a right-handed thread or counter clockwise if the threadform 572 is arranged in a left-handed thread) to draw the tapered portion through the fat layer (when rotated prior to reaching the fascia 127f) and through the fascia abdominal muscle 127f The distal tip of the dilator 570, having the smallest outside dimension, can enter the opening through the fascia 127f by slight pushing (and manipulation such as “wigging”) on the dilator 570, for example. By further rotating the dilator, the blunt edged threadform 572, threads its way into and through the fascia/abdominal muscle 127 without cutting it, but drawing the tapered portion of the dilator 570 along with it, thus gradually dilating the opening in the fascia 127f Thus, the threadform 572 provides mechanical advantage for enlarging the opening through the fascia/abdominal muscle 127f without cutting, but rather by dilating. Alternatively, the tapered surface of the dilator 570 between the threads could have a texture like a file, which would serve to help break the facial tissues during dilation. FIG. 9C illustrates dilator 570 being turned to draw the tapered portion 570t through the fascia 127f via the action of the threadform 572 on the fascia 127f.

Continued turning of the dilator 570 continues the drawing of the dilator 570 through the hole in the fascia 127f and or abdominal muscle. A large cannula 310L can be slid over the non-tapered portion of dilator 570 (or be pre-mounted thereon) to follow the dilator 570 as it is drawn in through the opening in the fascia, as illustrated in FIG. 9D. Large cannula 310L may have a tapered distal tip 310t that facilitates it following the dilator 570 through the opening in the fascia 127f In addition, the large cannula 310L may also have threadforms similar to the thread forms 572 on the dilator. Once large cannula 310L has been successfully placed through the opening and across the walls of the fascia and or abdominal muscle, dilator 570 can be slid out of large cannula 310L and therefore out of the patient leaving the cannula 310L and guide 530 in place, as illustrated in FIG. 9E. If endoscope 330 was removed during the dilation process illustrated in FIGS. 9B-9D, it may then be reinserted into guide 530, if desired by the surgeon during the part of the process illustrated in FIG. 9E. Alternatively, guide 530 can also be removed along with dilator 570 at this stage, leaving only the cannula 310L extending through the opening in the fascia, as illustrated in FIG. 9F. This will depend upon whether it is desired to view with an endoscope 330 inserted into guide 530 as it extends alongside another tool or implantable device advanced along the tract or if an endoscope is to be used in another tool extended along the tract. Further alternatively, other visualization schemes may be used, during which the guide 530 may be removed from the patient. while the example of FIGS. 9A-9F has been directed to dilating an opening in the fascia and or abdominal muscle, it is again emphasized here that neither the dilator nor any of the other tools and devices described herein are limited to placement through the fascia of the abdominal cavity, but may be used through other openings in the body, such as openings made by cutting, puncture or the like.

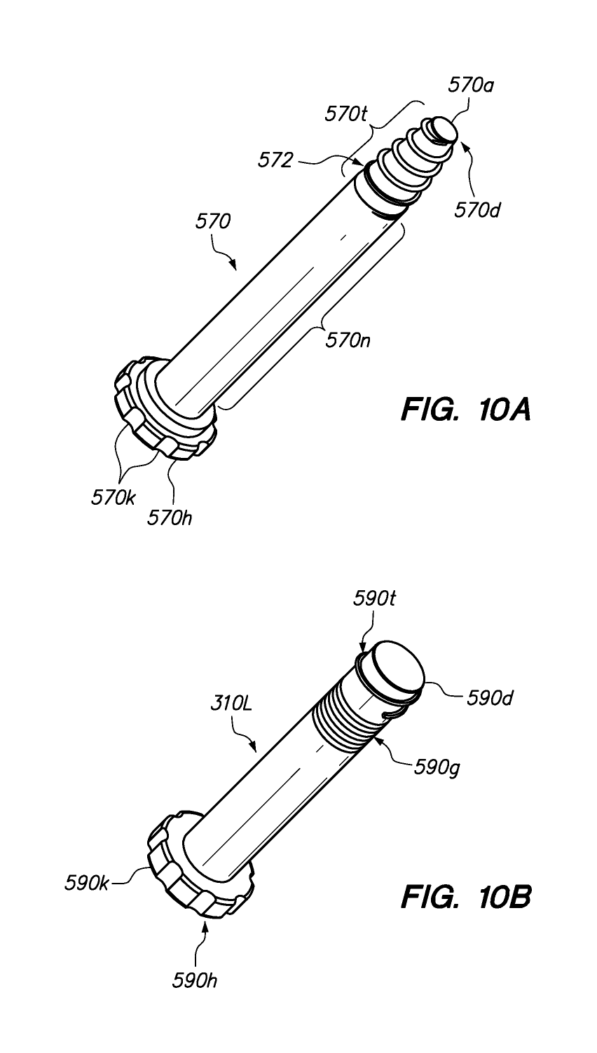

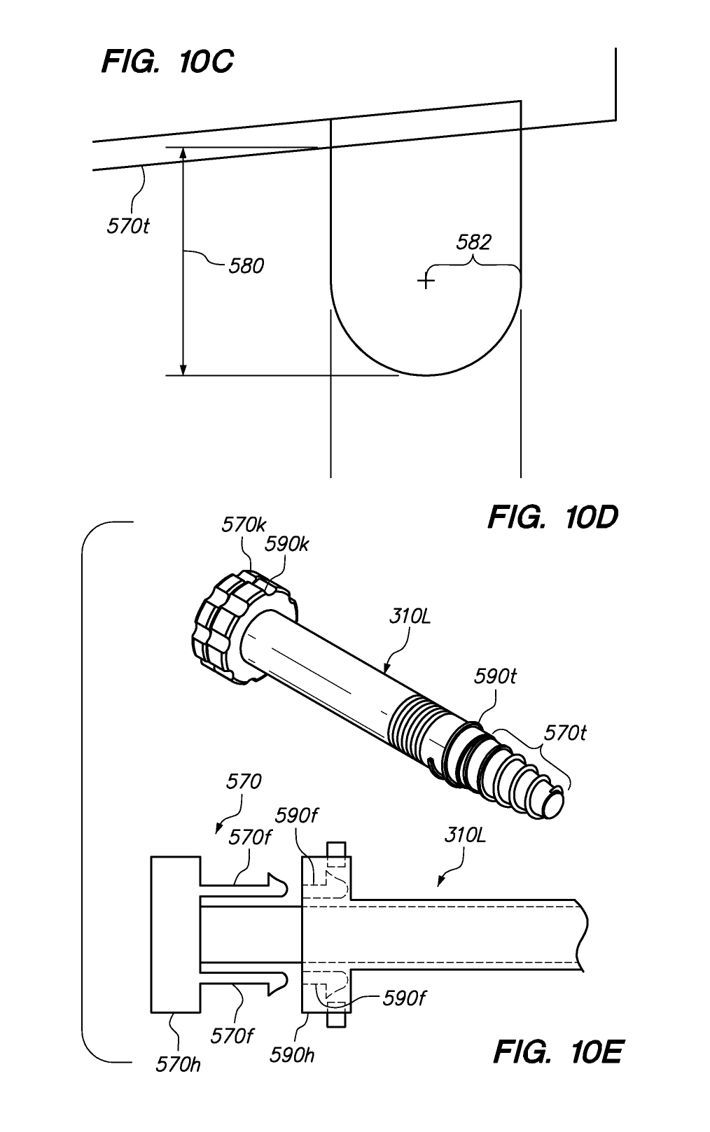

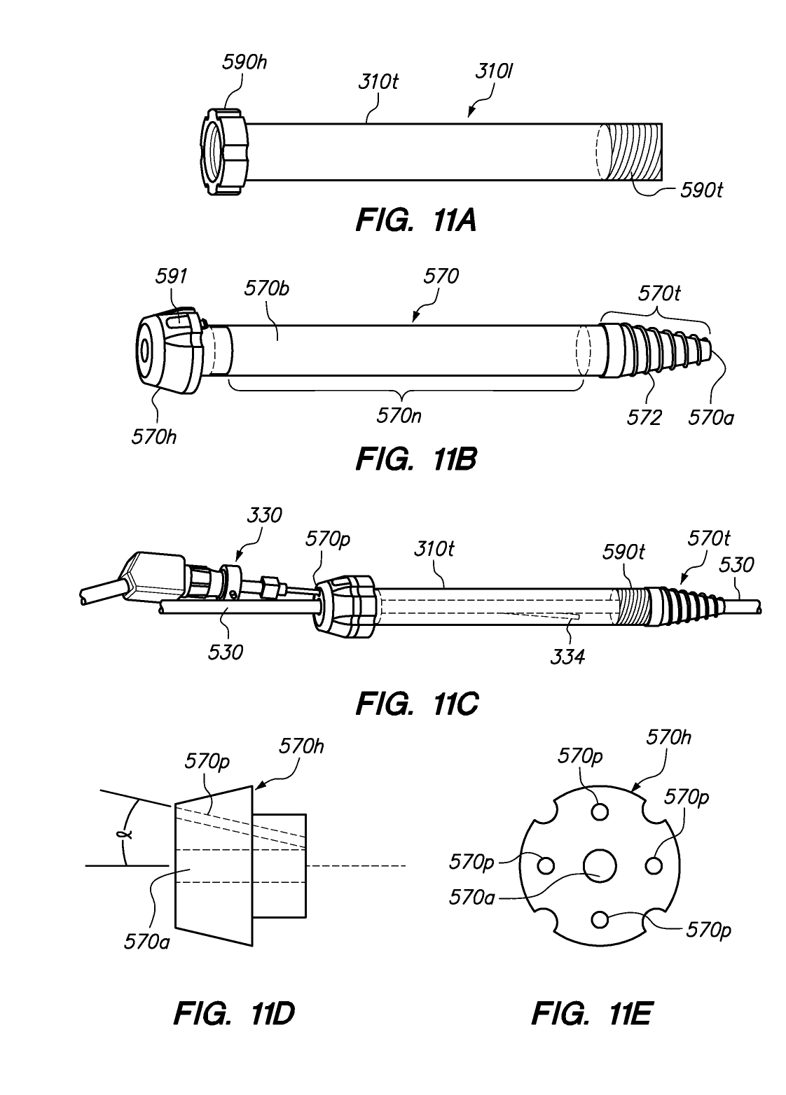

FIGS. 10A-10E show another embodiment of a dilator 570 and large cannula or introducer 310L that can be used in any of the same manners described above with regard to the dilator 570 and large cannula 310L described previously with regard to FIGS. 9A-9F, including use for delivery and placement of a conduit through which an implantable device and or tool can be delivered to a target surgical location. The tools of FIGS. 10A-10E, like those of FIGS. 9A-9F, can be made from one or more of the following materials: polycarbonate, glass-filled polycarbonate, glass-filled nylon, Grilamid® (semi-lubricious nylon product) Grivory® (semi-lubricious nylon product), polyetheretherketone (PEEK), Teflon® (polytetrafluoroethylene) and or Delrin® (acetal resin) or other injection molded, biocompatible plastic.

Like the embodiment of FIG. 9A, the dilator 570 of FIG. 10A is tapered, with a large threadform 572 along the tapered portion 570t and transitioning to the non-tapered portion 570n. In at least one embodiment the threadform 572 is about 1.5 threads per inch, has a pitch of about 2.67 and wherein the tapered portion has a taper of about eight degrees. In another embodiment, the threadform 572 is about 2.67 threads per inch, has a pitch of about 0.375 and the tapered portion has a taper of about eight degrees. Each of these specification may vary, but the threadform should remain large (e.g. about 1.1 to about 3.3 threads per inch) and the threads should extend sufficiently from the surface of the taper, e.g., about 0.065″ to about 0.125″, typically about 0.080″, but be blunt (rounded) so as to grab the tissues to dive the dilator into the abdominal cavity as the dilator 570 is rotated, without cutting the tissues that the threadform 572 contacts. Dilator 70 has a central annulus or lumen 570a extending therethrough which has a diameter slightly larger than the outside diameter of guide 530. Accordingly, annulus 570a may have a diameter of about 0.5″ or slightly larger. In one particular embodiment, dilator 570 has an inside diameter of about 0.505″ formed by annulus or lumen 570a, and an outside diameter of the non-tapered portion is about 0.995″. In another embodiment, the outside diameter of the non-tapered portion is about 1.588″ and the inside diameter is about 0.505″. The distal end of dilator 570, where the tapered portion begins has an outside diameter of slightly greater than the annulus diameter, e.g., about 0.6″ to about 0.7″ and tapers to the cross-sectional dimension of the non-tapered section 570n, which may, for example, have an outside diameter of about 1.0 inches to about 1.7 inches. In another example, the outside diameter of the non-tapered portion 570n was about 1.2 inches. The profile of the threadform 572 can be radiused so that there are no sharp edges on the threadform 572, thereby greatly reducing the risk of trauma. Dilator 570 (including threadform 572) may be made of a relatively rigid, but lubricious polymer, such as DELRIN® (acetal copolymer) or other acetal copolymer, or other suitable biocompatible polymer, such as an injection moldable polycarbonate with or without a radiopaque filler or marker band. FIG. 10C illustrates one specific embodiment of a threadform 572 that extends from the surface of the taper 570t by a distance 580 of about 0.080 inches and wherein the free or exposed edge of the threadform 572 has a radius of curvature 582 of about 0.030″.

Dilator 570 has a central annulus or lumen 570a extending therethrough which has a diameter slightly larger than the outside diameter of guide 530. Accordingly, annulus 570a may have a diameter of about 0.5″ or slightly larger. In one particular embodiment, dilator 570 has an inside diameter of about 0.505″ formed by annulus or lumen 570a, and an outside diameter of the non-tapered portion is about 0.995″, with a length of the overall dilator 570 being about 8.7″. In another particular embodiment, the inside diameter and length were the same, but the outside diameter of the non-tapered portion 570n was about 1.060″. In still another embodiment, the inside diameter is the same, but the length of the dilator 570 is about 16.16″ and the outside diameter of the non-tapered portion 570n is about 1.588″. Thus, the inside diameter of dilator 570 at the distal end 570d closely matches the outside diameter of tube 534 being only slightly larger (e.g., about 0.005″±about 0.002′) to allow free sliding of the dilator 570 over the guide 530, but fitting closely to prevent this interface from grabbing tissues as the dilator 570 is advanced over guide 530. The distal end of dilator 570, where the tapered portion begins has an outside diameter of slightly greater than the annulus diameter, e.g., about 0.6″ to about 0.7″ and tapers to the cross-sectional dimension of the non-tapered section 570n, which may, for example, have an outside diameter of about 0.8 inches to about 1.7 inches.

In FIG. 10A, dilator 570 additionally includes an enlarged handle 570h at a proximal end thereof that is configured to be grasped by a user to facilitate an increase in the amount of torque the user can apply to the dilator 570 by rotating handle 570h. Thus, handle 570h has a larger outside diameter than the non-tapered cylindrical portion 570n of dilator 570. Further, handle 570h can be provided with knurls 570k or other features that render handle 570h less smooth or otherwise increase friction, to prevent the user's hand from slipping during torquing.

The large cannula 310L of FIG. 10B is configured to slide over dilator 570 with a close, but freely sliding fit (e.g., inside diameter of large cannula 310L is about 0.005″±about 0.002″ greater than outside diameter of portion 570n) and large cannula 310L has a length such that when handle 590h contacts handle 570h, the threaded, tapered portion 570t of dilator 570 extends distally of the distal end of large cannula 310L as shown in the assembled view of FIG. 10D. In another embodiment, the close, but freely sliding fit is provided wherein the inside diameter of large cannula 310L is about 0.012″±about 0.005″ greater than outside diameter of portion 570n In one embodiment where the dilator had a length of about 8.67″, and inside diameter of about 0.505″ and the portion 570n had an outside diameter of about 0.995″, the large cannula 310L had a length of about 6.375″, an inside diameter of about 1.055″ and an outside diameter of about 1.105″. In another embodiment where the dilator had a length of about 16.16″, and inside diameter of about 0.505″ and the portion 570n had an outside diameter of about 1.588″, the large cannula 310L had a length of about 11.855″, an inside diameter of about 1.610″ and an outside diameter of about 1.690″. In another particular embodiment the dilator had the a length of about 8.67″ and the same inside diameter as the previous embodiments, but an outside diameter of about 1.060″ and the large cannula had a length of about 6.375″, an inside diameter of about 1.065″ and an outside diameter of about 1.115″. In all embodiments, the inside diameter of large cannula 310L forms a close fit with the outside diameter of the cylindrical portion 570 to allow free sliding between the components, but to prevent snagging of tissue between the distal end of large cannula 310L and dilator 570 as these components are inserted into the body. The distal end portion of large cannula 310 L may comprise a radiopaque material or may be provided with a radiopaque feature for enhanced visibility under fluoroscopy. Likewise, the distal end portion of dilator 570 may comprise a radiopaque material or may be provided with a radiopaque feature for enhanced visibility under fluoroscopy.

Large cannula 310L may be provided with a first threadform 590t that matches the pitch of the threadform 570t and extends from the surface of the cylindrical main body of large cannula 310L by a distance equal or similar to the distance that threads 570t extend from the conical portion of the dilator 570. In this way, threads 590t can be aligned with threads 570t so that the threadform 590t acts as a continuation of threadform 570t by extending smoothly and substantially continuously therefrom as illustrated in FIG. 10D. However, it is not critical that the threads 570t and 590t are aligned in this manner, as threads 590t can start independently of the thread 570t after the thread 570t has passed through the fascia or other opening being enlarged. Further alternatively, the threads 590t may, but need not match the thread height of the thread 570t of the dilator 570. The threads 590t of the large cannula 310L can alternatively have a different threadform and pitch than threads 570t of the dilator 570. In one embodiment where the height of threads 570t (measured from the peak of the thread to tapered outer surface of tapered portion) was about 0.085″, the height of threads 590t (measured from the peak of the thread 590t to the non-threaded surface of the large cannula 310L) was about 0.065″. The threads 590t can be alternatively replaced by a series of spaced, parallel ribs that extend around the circumference of the introducer in a direction substantially normal to the longitudinal axis thereof, or such ribs can be provided in addition to the threads 590t. To assist in alignment of the threads 570t, 590t and maintenance of the alignment handle pattern 590k is provided that both assists grip by the user, and matches up with the pattern 570k on the handle 570h of the dilator. Accordingly, as shown in FIG. 10D, when threads 570t are aligned with threads 590t the knurling pattern 590k aligns with knurling pattern 570k. By maintaining alignment of the patterns 570k, 590k (the user can maintain alignment by grasping both 570k and 590k in his or her hand) during torquing, threads 570t, 590t can be seamlessly threaded in through an opening, e.g., in the fascia, muscle, diaphragm or other tissue.

Alternatively or additionally, handle 570h may be provided with at least one fastening component 570f and handle 590h may be provided with at least one mating fastening component 590f, one for each respective fastening component 570h. As shown in FIG. 10E, handle 570h includes two male fastening components 570h and handle 590h includes two corresponding mating female components 590f However, one or more than two such components may be provided on handle 570h and, correspondingly, in handle 590h. Further, the male components) can be provided on handle 590h and the female components can be provided in handle 570h. Still further, although bayonet couplings 570f and mating female receptacles 590f are shown, alternative mating components may be used, such as shafts with ball and detent arrangements, or any of a number of mating, releasable mechanical fixtures. The mating mechanical members 570h and 590h, when connected, maintain the large cannula 310L fixed relative to the dilator 570, both in the axial direction, as well as rotationally. Accordingly, these fixtures can be arranged so that when they are connected together, the threads 570t and 590t are aligned, and the distal end of the large cannula 310L is properly axially aligned with the distal end portion of the dilator 570 as intended. A release mechanism 591 may be provided that the user can actuate, once the cannula 310L has been properly positioned so that the distal portion including threads 590t has been threaded through the opening in the fascia, to release the mechanical fixation member 570h, 590h and then the operator can remove the dilator 570 from the large cannula 310L and the patient by withdrawing on handle 570h while holding handle 590h stationary relative to the patient. In the example shown in FIG. 10E, the release mechanism 591 comprises a pair of release buttons 591 that the operator can press on to release the bayonet male members 570f from the receptacles 590f Handles 570h, 590h can have substantially the same size/outside diameter, as shown in FIG. 10D, but this is not necessary.

The distal end 590d of large cannula 310L may be chamfered so that it tapers towards the dilator 570 when assembled thereover, thereby further reducing the risk of snagging tissue (e.g., fascia) as the tools are threaded into the body. Alternatively, the tip 590d may be flexible and tapered to a smaller diameter to create intimate contact and smooth transition with the dilator 570. In this embodiment, the tip 590d could be composed of an elastomeric material or a more rigid material where the tip 590d is radially interrupted to allow the stiffer material to flex radially outwards to allow an interference fit that slides under low force. This same type of transition could be applied to the dilator tip 570d, to provide a smooth transition to the guide tube 530. In addition to aiding in the dilation procedure, threads 590d provide tactile feedback to the user to let the user know when the distal end of large cannula 310L has been threaded into the abdominal cavity through the hole in the fascia, as the user can feel the cannula 310L being drawn in through the hole in the fascia by the threads 590t as the cannula 310L is rotated. Further, the threadforms allow the user to feel when they have passed through the facial hole such that the large cannula 310 can then translate forward more easily. This tactile feedback allows the user to feel when the end of the large cannula 310 has appropriately passed beyond the fascia. Further, the distal threads 590t on the introducer 310L are configured to help prevent the large cannula 310L from accidentally pulling out of the abdominal cavity. Coarse ridges 590g may be provided on the distal end portion of large cannula 310L proximal of threads 590t. The coarse ridges 590g function to increase friction between them and the surrounding tissues to help prevent movement of the large cannula 310L relative to the patient's body, once it has been inserted in the desired position. As shown, the coarse ridges are parallel to one another and closely spaced. Once the distal end portion of large cannula 310L has been installed through the opening in the fascia, dilator 570 can be withdrawn from the cannula 310L and the patient 1 leaving the large cannula 310L in place to provide access to the abdominal cavity by tools and/or implants. Guide 530 may also be left in place to guide tools and/or implants. Alternatively, guide 530 may be removed to provide greater cross-sectional area of the large cannula 310L, such as for insertion and use of one or more tools and or implantable devices.

FIGS. 11A-11C show another embodiment of a dilator 570 and large cannula or introducer 310L that can be used in any of the same manners described above with regard to the dilator 570 and large cannula 310L described previously with regard to FIGS. 9A-9F as well as the embodiment described with regard to FIGS. 10A-10E, including use for delivery and placement of a conduit through which an implantable device and/or tool can be delivered to a target surgical location. In the embodiment shown in FIG. 11A, large cannula/introducer 310L includes a transparent main body tube with a handle portion 590h and may include threads 590t and/or ribs on the distal end portion thereof. Like the previous embodiments, the handle 590h and distal end portion of introducer 310L in FIG. 11A are opaque, but alternatively, can be transparent.

Like the previous embodiments, the dilator 570 of FIG. 11B is tapered, with a large threadform 572 along the tapered portion 570t and transitioning to the non-tapered portion 570n. Like the previous embodiments, the angle of taper of the outer surface of the tapered portion 570t relative to a central longitudinal axis of the dilator 570 is in the range of about seven degrees to about 13 degrees, typically about eight degrees to about 12 degrees. In one embodiment the angle was about 10.5 degrees (or 21 degrees measured from outer surface to opposite outer surface of the cone).

In this embodiment non-tapered portion 570n is transparent. Tapered portion 572 is opaque, like in previous embodiments. Dilator 570 has a central annulus or lumen 570a having at its distal end a diameter slightly larger than the outside diameter of guide 530. Accordingly, annulus 570a may have a diameter of about 0.5″ or slightly larger. Annulus 570a expands to an enlarged annulus 570b within the non-tapered portion that is only slightly smaller than the inside diameter of tube 310t.

Handle 570h fits in the annulus 570b to close the proximal end thereof Handle 570h extends the annulus 570b via annulus 570a, which is the same dimension of the annulus 570a at the distal end of dilator 570 and therefore closely follows over guide 530. Additionally, handle 570h may be provided with one or more endoscope ports 570p dimensioned and configured to allow an endoscope 330 (typically a rigid endoscope) to be inserted therethough, such that the endoscope shaft 332 and tip 334 are inserted at an angle α relative to the longitudinal axis of the handle 570h and dilator 570. In one embodiment, port 570p has a diameter of about 0.295″ to about 0.305″ (about 7.62 mm) to allow for insertion of a five mm endoscope shaft therethrough. These dimensions may vary, as the dimension of the endoscope shaft to be received may vary. Angle a may range from about twenty degrees to about seventy degrees, or from about twenty-five degrees to about forty-five degrees. In the embodiment shown in FIG. 11D, a is about thirty degrees. When providing multiple endoscope ports 570p, the multiple ports may each be provided at the same angle α and simply located at different angles (i.e., “clock” positions) about the circumference of the handle 570h. Alternatively, one or more ports 570p may be formed at different angles a relative to the longitudinal axis of the handle. This may also cause ports 570p to have varying radial distances from the central axis of lumen 570a, as shown in FIG. 11E. With the angles that are used, the endoscope shaft 332 bypasses the inside surface of handle 590h without contacting it, so that handle 590h does not have to be modified from previously described embodiments.

By inserting endoscope 330 through port 570p in the manner exemplified in FIG. 11C, the surgeon can view the anatomy by viewing through the tubes 570n and 370t. Thus, for example, in a situation like shown in FIG. 9D, the surgeon would be able to view the fascia 127f through endoscope 330 and ascertain whether or not the dilator 570 has successfully passed through the fascia.

The transparent tube 310t and 570n can be extruded parts (e.g., polycarbonate) and the opaque components 590h, 590t 570t and 570h can be molded from polycarbonate.

In one particular embodiment dilator 570 has an inside diameter of about 0.505″ formed by annulus or lumen 570a, and an outside diameter of the non-tapered portion is about 0.995″, with a length of the overall dilator 570 being about 8.7″. In another particular embodiment, the inside diameter and length were the same, but the outside diameter of the non-tapered portion 570n was about 1.060″. In still another embodiment, the inside diameter is the same, but the length of the dilator 570 is about 16.16″ and the outside diameter of the non-tapered portion 570n is about 1.588″. Thus, the inside diameter of dilator 570 at the distal end 570d closely matches the outside diameter of tube 534 being only slightly larger (e.g., about 0.005″±about 0.002″) to allow free sliding of the dilator 570 over the guide 530, but fitting closely to prevent this interface from grabbing tissues as the dilator 570 is advanced over guide 530. The distal end of dilator 570, where the tapered portion begins has an outside diameter of slightly greater than the annulus diameter, e.g., about 0.6″ to about 0.7″ and tapers to the cross-sectional dimension of the non-tapered section 570n, which may, for example, have an outside diameter of about 0.8 inches to about 1.7 inches.

FIG. 12 illustrates an embodiment of a conduit 600 that can be inserted through large cannula 310L to extend distally far past the distal end of large cannula 310L, for delivery of one or more tools and or implants therethrough, to a surgical target location, such as in the abdominal cavity, in the thoracic cavity, in an internal organ or other internal location in the body where implantation of one or more devices or performance of one or more surgical procedures not requiring an implant is to be accomplished. The location can actually be quite shallow, relative to skin lying directly over it such as a location along the fascia or ribs. However, the location is “far” in the sense that it located away from the opening through the skin by a relatively large distance, a distance that is significantly greater than the length of the large cannula 310L, as noted above. Of course, the location can, alternatively, be located deep within the body of the subject. The length of conduit 600 is typically at least about 1.5 times the length of large cannula 310L, and may be at least 2 times, at least 2.25 times, at least 2.5 times or at least 3 times the length of large cannula 310L The embodiment of FIG. 12 is formed of relatively rigid plastic. hi one embodiment this relatively rigid conduit 600 had a length of about 28.25 inches, an inside diameter of about 1.00 inches and an outside diameter of about 1.05 inches. In another embodiment this relatively rigid conduit 600 had a length of about 24.325 inches, an inside diameter of about 1.425 inches and an outside diameter of about 1.05 inches. Conduit 600 may include a chamfered or otherwise tapered distal end 600d so that it tapers towards the obturator 630 when assembled thereover, thereby reducing the risk of snagging tissue as the tools are inserted into the abdominal cavity, and generally helping to keep fluids and other tissues out of the conduit 600 as it is being advanced. Further optionally, the tapered distal end 600d may compress against the distal tip of the obturator 630 and/or form an interference fit therewith, preventing the distal tip of the obturator 630 from passing therethrough so that the obturator 630 be used to push against the conduit 600 via this contact to drive the conduit into the abdominal cavity and prevent the distal end of the conduit 600 from compressing or buckling toward the proximal end of the conduit 600. This fit between the distal end 600d and distal tip of the obturator 630 can also effectively seal the contact between the tapered distal end 600d and the distal end part/distal tip of the obturator 630, thereby preventing fluid inflow and tissue ingress into conduit 600 as it is advanced.

A flared or funnel portion 602 may be provided, either integrally with or attached to the proximal end portion of conduit 600. A seal 604 such as an o-ring may be provided to seat with the proximal end portion of the obturator 630 or proximal end of a tool. Further, a grasping tab 606 may be provided that can be pulled by the user to remove a perforated strip from the funnel portion 602 to expose slot 608. In instances where funnel portion 602 and the proximal end portion of conduit 600 are flexible, this allows deformation of the funnel portion 602 and proximal end portion of the conduit along slot 608 to allow a shaft handle or tube that extends transversely from a tool (e.g., light post of an endoscope, handle 412t of tool 400, etc.) to slide therealong, thereby reducing the effective length of the tool 400, endoscope 330 or other tool that needs to be provided to enable a distal end thereof to extend distally of the conduit 600. In embodiments where funnel portion 602 (and optionally, the proximal end portion of conduit 600) are rigid, the funnel portion 602 and adjoining proximal end portion of conduit 600 can be provided as half pieces that are hinged together, wherein a pair of opposing separations are formed between the halves (one in the location of and replacing slot 608 and one at a location about 180 degrees from there) to allow separation of the funnel portion 602 and proximal end portion.

FIGS. 13A-13C illustrate another embodiment of a conduit 600 in which at least a distal end portion thereof is flexible. In this embodiment the main tube of the conduit is formed of an elastomer, such as silicone, and a coil 610, such as a stainless steel coil, Nitinol coil, or the like, is encapsulated in the elastomer along at least the distal end portion of the conduit 600. Note that the chamfered or tapered distal end 600d is not reinforced with the coil 610. At least a 4″ length of the conduit 600 extending proximally from the unreinforced distal end 600d is reinforced with coil 610. In other embodiments, a least a quarter or at least a third or at least half of the length of the conduit 600 extending proximally from the unreinforced distal end 600d is reinforced with coil 610. In the example shown in FIG. 13A and the sectional view of FIG. 13C, coil 610 reinforces more than half of the entire length of the main body tube of conduit 600, extending proximally from the unreinforced distal end 600d. In still other embodiments, coil 610 may extend proximally from unreinforced distal end 600d and support the entire length of the tube up to the distal end of slot 608. In embodiments where slot 608 is not present, coil 608 may reinforce the entire length of the tube of conduit 600, but typically not the tapered distal end 600d or funnel portion 602. Portions of the main tube of conduit 600 that are proximal of the proximal end of coil 610 maybe made of an alternative material, such as a rigid polymer, so that this portion of the conduit is not flexible. Alternatively, portions of the main body of conduit 600 that are proximal of the proximal end of coil 610 may be flexible. Further alternatively, the main body of the conduit 600 can have no coil reinforcement but instead have reinforcements running longitudinally to allow bending but prevent stretching and/or bucking.

The reinforcement provided by coil 610 helps preserve the substantially circular cross section of the conduit 600 as it bends along a portion supported by coil 610, and coil 610 serves to prevent kinking along a supported portion as it is bent. In one particular embodiment a conduit of the type described with regard to FIGS. 13A-13C had a length of about 28.25 inches, an inside diameter of about 1.00 inch and an outside diameter of about 1.060 inches. In another particular embodiment, a conduit of the type described with regard to FIGS. 13A-13C had a length of about 24.325 inches, an inside diameter of about 1.425 inches and an outside diameter of about 1.505 inches.

In at least one embodiment where the funnel portion 602 is flexible, a notch 608n may be molded into the funnel portion 602 and proximal portion of tube 600 to produce a thinner portion along the line formed by notch 608n to facilitate a controlled tear of the material over a predefined length that is defined by the length of notch 608n. In the enlarged partial views of FIGS. 13D and 13E, notch 608n is formed as a triangular-shaped (in cross-section) notch and the thinner material portion can be seen at 608t in FIG. 13E.

At least the inside surfaces of conduit 600 may be coated with a lubricious coating such as a hydrophilic coating or other lubricious coating to reduce friction between an implant, device or tool inserted therethrough as it is delivered toward the surgical target location. In at least one embodiment, the lubricious coating comprises LUBRILAST™ (AST Products, Inc., Billerica, Mass.), e.g., see U.S. Pat. No. 6,238,799, which is hereby incorporated herein, in its entirety, by reference thereto. Additionally, at least a portion of the outside of conduit 600 may also be coated with a lubricious coating, which may be the same as the inside coating, for example.

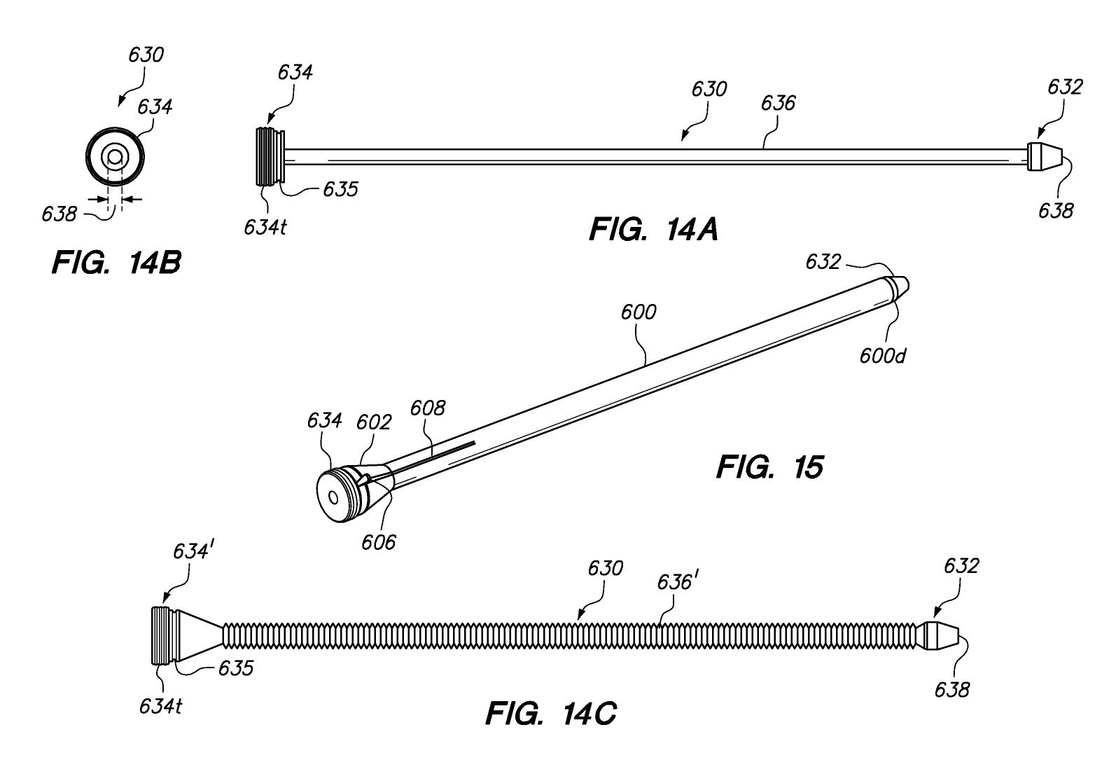

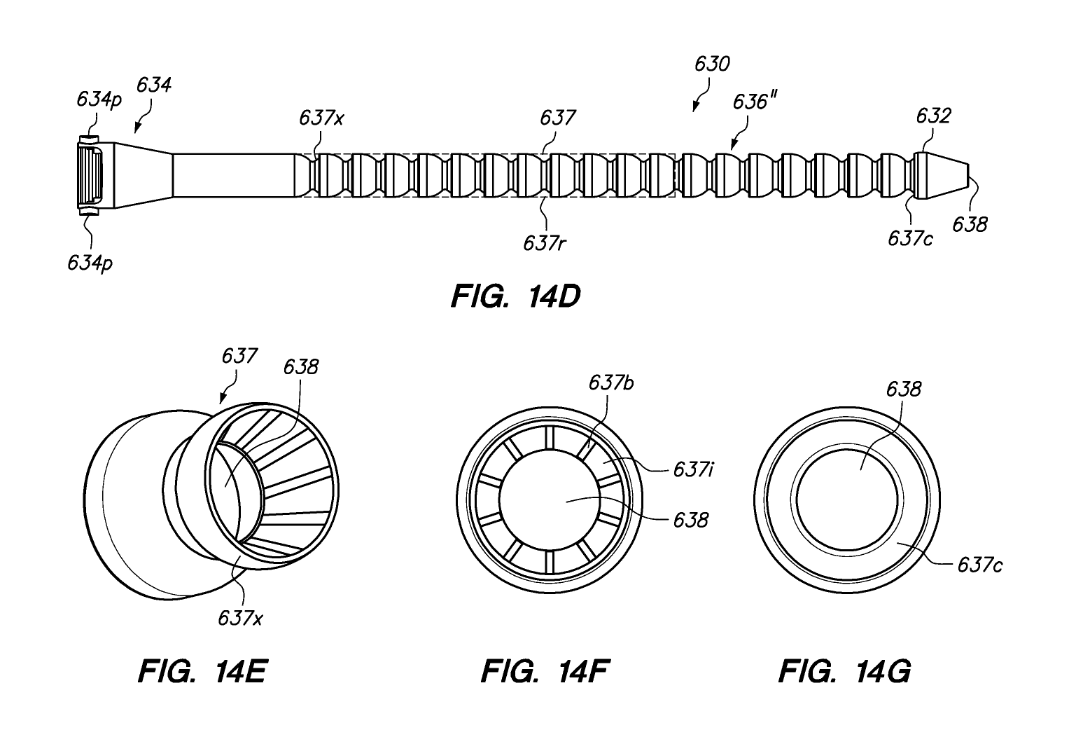

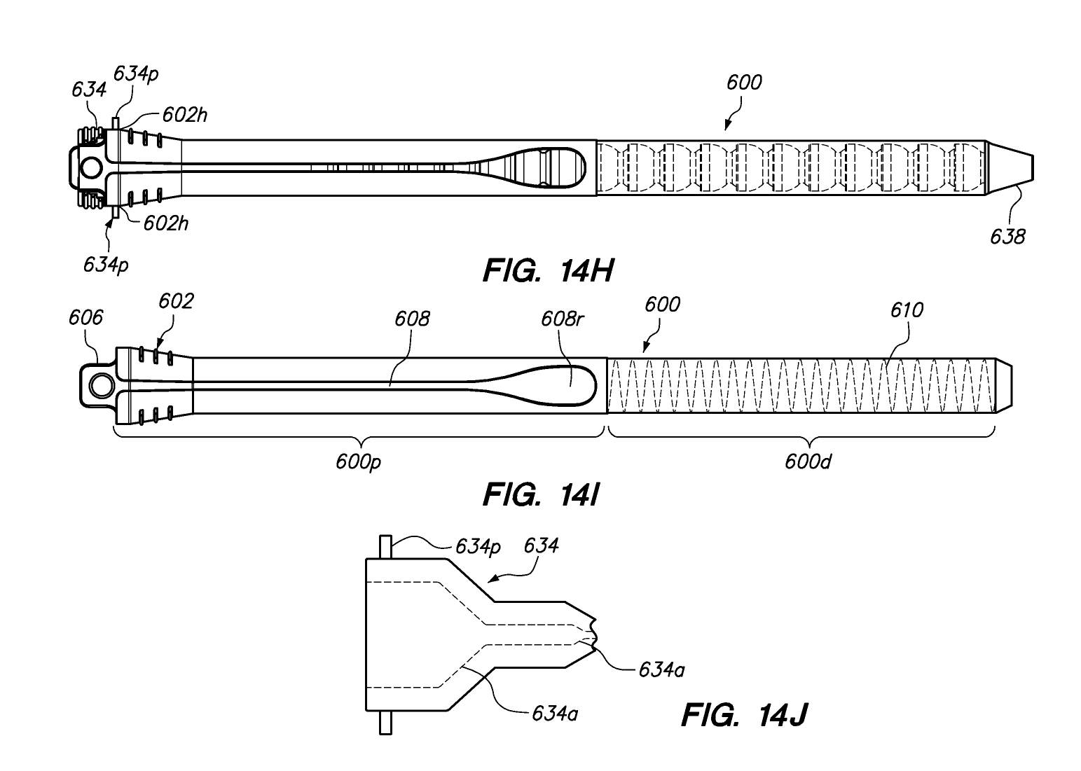

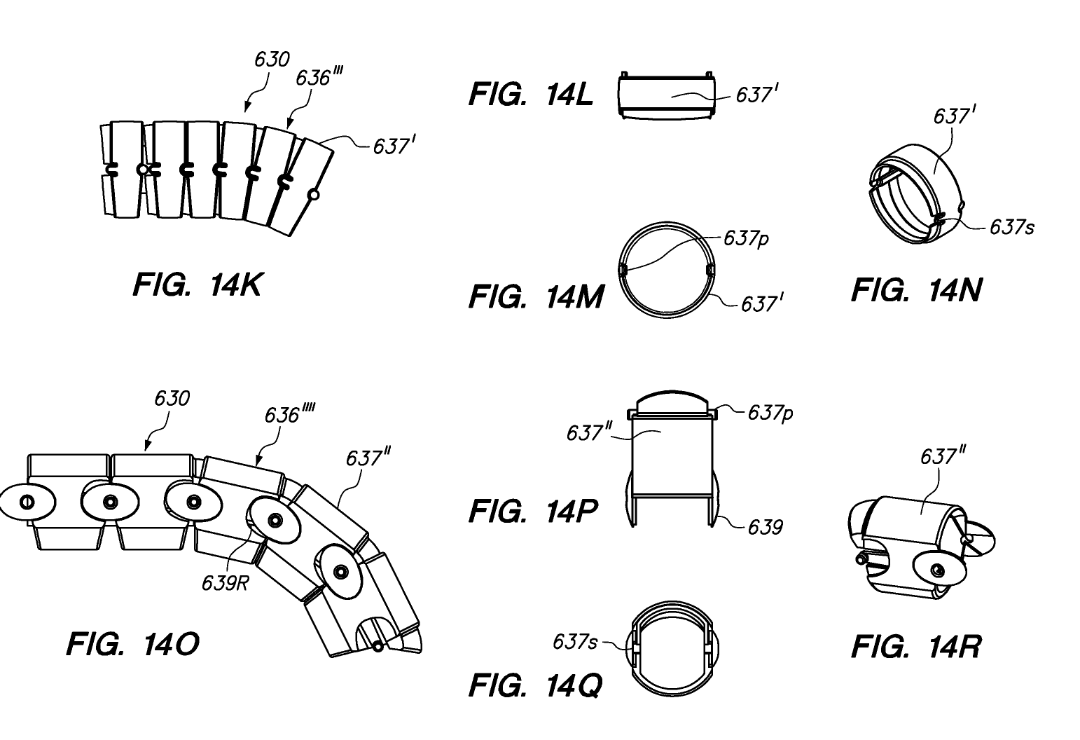

FIGS. 14A-14B illustrate a plan view and a proximal end view of an obturator 630 that is configured to be placed in conduit 600 and used to deliver conduit 600 through large cannula 310L and over guide 530 to deliver a distal end portion of conduit 600 far distally of the large cannula 310L. Obturator 630 has a length slightly greater than the length of conduit 600 so that when the tapered portion of distal tip 632 contacts chamfered end 600d, the handle 634 at the proximal end of obturator 630 extends slightly proximally of the proximal end of conduit 600 or the proximal end of funnel portion 602 when provided at the proximal end of conduit 600. Handle 634 and distal tip 632 are typically rigid and may be injection molded from hard plastic. Shaft 636 is relatively flexible and may be formed of extruded PEBAX® (polyether bock amides) or similar lubricious polymer extrusion that facilitates it sliding over guide 530 or may have a corrugated geometry or an interrupted linked geometry to allow flexibility.

A textured surface 634t such as grooves or the like may be provided on handle 634 to enhance grip by a user, as well as interfacing with seal 604. In one particular embodiment obturator 630 had an overall length of about 29.64″, an inside diameter 638 (see proximal end view of FIG. 14B) of about 0.505″, an outside diameter of shaft 636 of about 0.565″, an outside diameter of distal tip 632 of about 0.995″ and an outside diameter of handle of about 1.880″. In another particular embodiment, obturator 630 had an overall length of about 26.307″, an inside diameter 638 (see proximal end view of FIG. 14B) of about 0.505″, an outside diameter of shaft 636 of about 0.565″, an outside diameter of distal tip 632 of about 1.375″ and an outside diameter of handle of about 1.950″.

A textured surface 634t such as grooves or the like may be provided on handle 634 to enhance grip by a user. Additionally, a groove 635 may be provided that is configured and dimensioned to receive the molded o-ring 604 so that o-ring 604 seats in groove 635. In one particular embodiment obturator 630 had an overall length of about 29.64″, an inside diameter 638 (see proximal end view of FIG. 14B) of about 0.506″ (for use with a guide 530 having an outside diameter of about 0.505″), an outside diameter of shaft 636 of about 0.565″, and an outside diameter of distal tip 632 (non-tapered portion) of about 0.995″ and an outside diameter of handle of about 1.880″.

FIG. 14C illustrates an alternative embodiment of obturator 630 in which shaft 636′is made of corrugated tubing. In one example, the corrugated tubing is fluorinated ethylene polypropylene (FEP) tubing, although alternative polymer materials may be used, e.g., polyethylene nylon, polypropylene, perfluoroalkoxy (PFA) copolymer, etc. Corrugated tubing shaft 636′ allows the conduit 600, when installed over the obturator 630, to take tight bends without kinking. The relatively large diameter of the obturator shaft 636,636′ also prohibits the conduit 600 from collapsing while the obturator 630 is installed in the conduit 600.

The obturator tip 632 may be an injection molded part and is provided with a central lumen annulus 638 configured and dimensioned to slide over the guide 530, while providing a close fit with the guide 530 to prevent tissues or other obstructions from entering between the obturator tip 632 and guide 530, as the obturator 630 having the conduit 600 assembled therewith is passed over the guide to deliver the distal end of the conduit 600 to the surgical target location.

Further alternatively, the obturator handle 634′ may be funnel-shaped or otherwise tapered to follow the tapered contour of the tapered portion 602 of conduit 600. The obturator handle 634,634′ may also be made of injection molded plastic. By providing the handle 634′ with a tapered section, this further enhances the ability of handle 634′ to prohibit the tapered portion 602 (when provided as a flexible component) from collapsing and inadvertently decoupling from the obturator 630. In one embodiment, obturator 630 had an outside diameter of shaft 636′ of about 1.380″ and obturator 630 had a length of about 24.438″, measured from the distal surface of boss 634p to the proximal end of the tapered surface of tip 632. The angle of an outer surface of the tapered distal tip 632 to the central longitudinal axis of the obturator 630 is in the range from about thirteen degrees to about nineteen degrees, making the angle of the cone formed by tip 632 twice that, or about twenty-six degrees to about thirty eight degrees. The obturator tip 638 may comprise radiopaque material to facilitate viewing it under fluoroscopy.

FIG. 14D illustrates an alternative embodiment of obturator 630 in which shaft 636″ is made of rigid inks 637. Rigid links 637 may be formed of glass-filled (10%, by weight) polycarbonate for example. Alternatively, links 637 can be made from polycarbonate, acrylonitrile butadiene styrene (ABS)-polycarbonate blend, glass-filled Nylon, Nylon (polyamides), polyethylene, ABS, polyether block amides (PEBA , polyetheretherketones (PEEK), liquid crystal polymers (LCP), stainless steel or other biocompatible metals, etc. Each rigid link 637 has a concave inner surface 637c formed in one end portion thereof and a convex outer surface 637x formed on an opposite end portion thereof In the preferred embodiment shown, the link 637 has the convex outer surface 637x formed on the distal end portion of the link 637 and concave inner surface 637c is formed in the proximal end portion of the link 637. However, this arrangement could be reversed, so that link 637 has the convex outer surface 637x formed on the proximal end portion of the link 637 and concave inner surface 637c is formed in the distal end portion of the link 637, as long as all links 637 are arranged in the same way (i.e., so that surfaces 637x are all either proximal or distal, and surfaces 637c are all in the opposite end portion).

Optionally, only the distal portion of obturator need be flexible and formed by links 637. Accordingly, a proximal portion can be alternatively be formed as a rigid extension 637r of handle portion 634 and may comprise at least a quarter, at least a third or about half of the length of the obturator, with the remaining distal portion be flexibly formed by links 637. Further alternatively, the proximal portion may be formed with a fewer number of links that are substantially longer than the links 637 in the distal portion, since the proximal portion does not need to be as flexible (or may not need to be flexible at all) and this could reduce costs of manufacturing, as well as reduce the potential amount of elongation under tension. Further alternatively links as shown in FIG. 14D can be fused together in the proximal portion so that they do not articulate with one another.

Links 637 snap together to form a series of connected links 637 as shown in FIG. 14D. The snap fittings are loose enough to allow the links 637 to freely rotate relative to one another, about the longitudinal axis of the obturator 630, as well as to pivot tend) relative to one another in any direction, 360 degrees about the longitudinal axis. However, the snap fittings maintain the connections between the links even under tensile forces at least up to twenty-two pounds, and in some embodiments up to about ninety-seven pounds. Likewise, the snap fitting connections maintain the connections between the links even under bending forces typically experienced during the uses described herein. Advantageously, since the links are relatively rigid , they do not stretch under tension or shorten under compression during use. Thus, the only change in length of obturator 630 of FIG. 14D during use (insertion into the body, as well as pulling the obturator out of the body) is due to the tolerances in the snap fittings between links 637, and this change is negligible for the purposes that the obturator is used, as described herein.

Surface 637x articulates with surface 637c to function like a ball joint, allowing the three-dimensional articulation ability described above. In the embodiment shown, the proximal end portion of link 637 includes a ribbed inner surface 637i having ribs 637b (see FIGS. 14E and 14F) that function to help direct the guide and keep it centered toward the central lumen annulus 638. Handle 634 is provided with ramped surfaces 634a that angle toward the central longitudinal axis of the handle and help guide the guide 530 therethrough, see FIG. 14J. Surface 637i (not considering ribs 637b, see FIG. 14F) can be concave, as shown, but need not be. FIG. 14O is an end view of link 637 (proximal end view for the embodiment shown) that shows the smooth surface provided by concave surface 637c that allows the convex surface 637x to articulate freely against. Note also, that in the embodiment of FIG. 14D, obturator tip 632 may be provided with an inner concave surface 637c (or outer convex surface 637x, depending upon the particular embodiment) to articulate with the distal-most link 637. Alternatively, tip 32 may be fixed to, or integral with the distal most link 637. Similarly, handle 634 may be provided with an outer convex surface 637x (or an inner concave surface 637c, depending upon the particular embodiment) to articulate with the proximal-most link 637. Alternatively, handle 634 may be fixed to, or integral with the proximal-most link 637. Handle 634 may further be provided with one or more pins (or bosses) 634p for temporarily securing a portion of the funnel 602, when portions of the funnel 602 are provided with through holes 602h that allow pins 634p to extend therethrough when the funnel portions are held on handle 634, as shown in FIG. 14H. The funnel portions can be peeled or pried away from pins 634p to allow obturator 630 to be withdrawn from conduit 600.

FIG. 14I shows the conduit 600 from FIG. 14H, without the obturator 630.

The distal portion 600d of conduit 600 is flexible (e.g., silicone, or the like) and reinforced with coil 610. Coil 610 is closed-wound or nearly closed-wound at the ends (e.g., the last two to five wraps, typically the last four wraps of each end) to allow the closed-wound wraps to be laser welded to each other to terminate the coil. To be closed-wound or nearly closed-wound, the coils must touch or be very close to each other to allow for the welding process. In between these closed-wound or nearly closed-wound coils, the coils are separated by gaps of about 0.012″ in one embodiment (although this may vary), as they are wound at about thirty-three wraps/inch with a 0/018″ diameter wire.

This construction facilitates the prevention of kinking and which also helps prevent buckling of the distal portion when under axial compression. Coil 610 may be made of stainless steel or other biocompatible spring wire or elastic material that is visible under fluoroscopy and will perform as described.

The proximal portion 600d of conduit 600 is rigid and includes funnel portion 602. In at least one embodiment rigid portion 602 is made from PEBAX. In at least one embodiment, rigid portion 602 is made from PEBAX having a hardness of 63A durometer. Slot 608 may be radiused 608R at its distal end for stress reduction to prevent cracking. Although the embodiment of FIG. 14I has only one slot 608, it may alternatively be provided with two or more slots 608 (e.g., a pair of oppositely located slots 608, or three or four circumferentially spaced slots or more). Conduit 600 may be provided with a lubricious coating (such as LUBRILAST™ of the like) to facilitate its passage through the large conduit 310L. Likewise, a lubricious coaling is provided over the interior of conduit 600 to facilitate insertion of obturator therein and withdrawal of obturator 630 therefrom. In one particular embodiment the main tube of obturator 600 had an outside diameter of about 1.595″, an inside diameter of about 1.425″ and a working length of about 22.65″ measured from the minimum diameter of the funnel portion 602 to the distal tip of the conduit 600, and a slit 608 length of about 13.3″.

Links 637 of obturator 630 allow the conduit 600, when installed over the obturator 630, to take tight bends without kinking. For example, for a conduit 600 having a working length of about 22.65″ and an inside diameter of about 1.425″, obturator 630, when installed in conduit 600 allows conduit 600 to be bent at a radius of curvature of at least about 2.5″ without kinking. The relatively large diameter of the links 637 and rigidity thereof, also prohibits the conduit 600 from collapsing while the obturator 630 is installed in the conduit 600. Although the conduit 600 is generally robust enough to prevent itself from kinking and collapsing, the links 637 may help the conduit 600 achieve a slightly tighter bend radius (about 10% smaller, for example). Links 637 only contact the inner wall of the conduit 600 at two point contacts per link or less. Many links 637 may not contact the conduit 600 at all. For example, in one embodiment, the inner wall of the conduit 600 has a diameter of about 1.425″ and the larges outside diameter of a link in this embodiment is about 1.259″. The small space between the obturator and the conduit is desirable because it minimizes tip shift between the obturator 630 and the conduit 600 during bending, but also provides enough room for the obturator to bend freely around the guide 530.

The obturator tip 632, handle 634 and links 637 may all be injection molded parts, e.g., injection-molded from polycarbonate or 10% glass-filled polycarbonate, or other alternative materials to 10% glass-filled polycarbonate that were listed above. Additionally, tip 632 may have 10% barium additive to make it radiopaque. The central lumen annulus 638 of obturator configured and dimensioned to slide over the guide 530, while providing a close fit with the guide 530 to prevent tissues or other obstructions from entering between the obturator tip 632 and guide 530, as the obturator 630 having the conduit 600 assembled therewith is passed over the guide to deliver the distal end of the conduit 600 to the surgical target location. Further alternatively, the obturator handle 634 may be funnel-shaped or otherwise tapered to follow the tapered contour of the tapered portion 602 of conduit 600. By providing the handle 634′ with a tapered section, this further enhances the ability of handle 634′ to prohibit the tapered portion 602 (when provided as a flexible component) from collapsing and inadvertently decoupling from the obturator 630.

Obturator 630 has a length slightly greater than the length of conduit 600 so that when the tapered portion of distal tip 632 contacts chamfered end 600d, the handle 634 at the proximal end of obturator 630 extends slightly proximally of the proximal end of conduit 600 or the proximal end of funnel portion 602 when provided at the proximal end of conduit 600. Like previous embodiments, a textured surface, such as grooves or the like may optionally be provided on handle 634 to enhance grip by a user. Further optionally, a groove may be provided that is configured and dimensioned to receive the molded o-ring 604 so that o-ring 604 seats in the groove.

FIG. 14K illustrates another alternative embodiment of obturator 630 in which shaft 636′″ is made of rigid links 637′. Rigid links 637′may be formed of thermosetting polymer and include slots 637s and projections 637p (see the side, end and perspective views of link 637′ in FIGS. 14L-14N, respectively) that snap together such that projections 637p of one link 637′ snap into slots 637s of an adjacent link and thereby allow relative rotation between projections 637p and slots 637s in only one plane. FIG. 14O shows another alternative embodiment of obturator 630, similar to the embodiment of FIG. 14K, in which shaft 636′″″ is made of rigid links 637″ that are longer than the links 637′ of FIG. 14K. Rigid links 637′ may be formed of thermosetting polymer and include slots 637s and projections 637p (see the side, end and perspective views of link 637′ in FIGS. 14P-14R, respectively) that snap together such that projections 637p of one link 637′ snap into slots 637s of an adjacent link and thereby allow relative rotation between projections 637p and slots 637s in only one plane. Disks 639 rotate and recesses 639R, whereby the boundaries of recesses 639R limit the amount of rotation of disks 639 relative thereto and thereby also limit the amount of relative rotation between links 637″ in the single plane.

In both the embodiments of FIGS. 14K and 14O, the rigid links 637′, 637″ are allowed to pivot relative to one another and the longitudinal axis of obturator 630, but can only pivot in one plane. Thus, obturator 630 can only bend left and right in a single plane and links 637′, 637″ are only able to articulate two-dimensionally. The restriction on articulation can provide an advantage as obturator 630 can then be used to help steer conduit 600 and even reposition guide 530, if needed, as torquing on obturator 630 (about the longitudinal axis thereof) while the obturator is bent will redirect the distal tip 632 of the obturator.

Links 637′, 637″ are loosely connected to allow free pivoting in the single plane, but connected with less loose tolerances out of the single plane to prevent pivoting in all directions out of the plane. Like the embodiment of FIG. 14D, since the links 637′, 637″are relatively rigid, they do not stretch under tension or shorten under compression during use. Thus, the only change in length of obturator 630 of FIG. 14H during use (insertion into the body, as well as pulling the obturator out of the body) is due to the tolerances in the joint connections between links 637′, and this change is negligible for the purposes that the obturator is used, as described herein.

Links 637′, 637″ allow the conduit 600, when installed over the obturator 630, to take tight bends in the plane of pivoting, without kinking. The relatively large size of the links 637′, 637″ and rigidity thereof, also prohibits the conduit 600 from collapsing while the obturator 630 is installed in the conduit 600.

The obturator tip 632, handle 634 and links 637′, 637″ may all be injection molded parts

The central lumen annulus 638 of obturator 630 is configured and dimensioned to slide over the guide 530, while providing a close fit with the guide 530 to prevent tissues or other obstructions from entering between the obturator tip 632 and guide 530, as the obturator 630 having the conduit 600 assembled therewith is passed over the guide to deliver the distal end of the conduit 600 to the surgical target location. Further alternatively, the obturator handle 634 may be funnel-shaped or otherwise tapered to follow the tapered contour of the tapered portion 602 of conduit 600. By providing the handle 634 with a tapered section, this further enhances the ability of handle 634 to prohibit the tapered portion 602 (when provided as a flexible component) from collapsing and inadvertently decoupling from the obturator 630.

Obturator 630 has a length slightly greater than the length of conduit 600 so that when the tapered portion of distal tip 632 contacts chamfered end 600d, the handle 634 at the proximal end of obturator 630 extends slightly proximally of the proximal end of conduit 600 or the proximal end of funnel portion 602 when provided at the proximal end of conduit 600. Like previous embodiments, a textured surface, such as grooves or the like may optionally be provided on handle 634 to enhance grip by a user. Further optionally, a groove may be provided that is configured and dimensioned to receive the molded o-ring 604 so that o-ring 604 seats in the groove.

FIG. 15 illustrates an embodiment of obturator 630 having been inserted into conduit 600. When the obturator embodiment of FIG. 14A is used, preferably, the contact between obturator 630 and conduit 600 occurs only between the distal tip 632 (tapered portion) and the chamfered end 600d, and between the funnel portion 602/seal 604 and the handle 634. This maximizes the ability of conduit 600 to make bends of the smallest possible bend radii, without kinking or distortion. However, the other embodiments of obturator typically do contact the conduit 600 at locations intermediate of the distal tip 632 and handle 634.

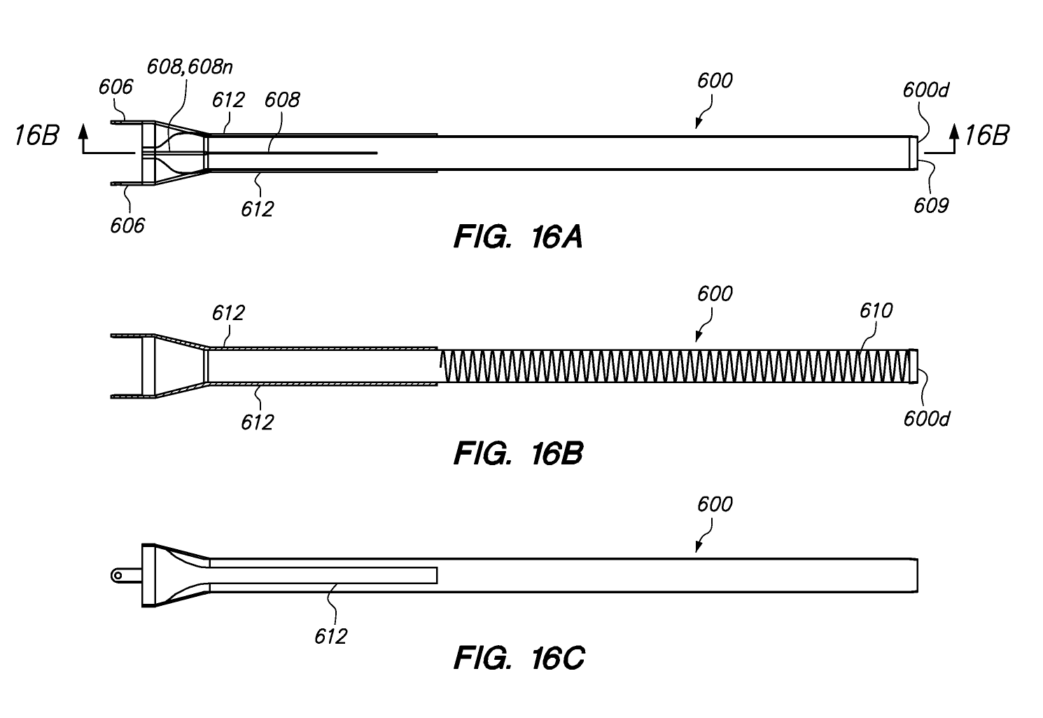

FIGS. 16A-16C illustrate an alternative embodiment of conduit 600 according to the present invention. Like the embodiment of FIGS. 13A-13E, the main tube of the conduit 600 is flexible and is formed of an elastomer, such as silicone, and a coil 610, such as a stainless steel coil, Nitinol coil, or the like, is encapsulated in the elastomer along at least the distal end portion of the conduit 600. Also like the embodiment of FIGS. 13A-13E, the chamfered or tapered distal end 600d is not reinforced with the coil 610. At least a 4″ length of the conduit 600 extending proximally from the unreinforced distal end 600d is reinforced with coil 610. In other embodiments, a least a quarter or at least a third or at least half of the length of the conduit 600 extending proximally from the unreinforced distal end 600d is reinforced with coil 610. In the example shown in FIGS. 16A-16C, coil 610 reinforces more than half of the entire length of the main body tube of conduit 600, and extends proximally from the unreinforced distal end 600d to a location distally adjacent the distal ends of stiffening members 612. The proximal end portion of the main tube of conduit 600 that is proximal of the proximal end of coil 610 is reinforced by one or more stiffening member 612 (two stiffening members 612, as shown, although one, or more that two stiffening members 612 may be employed). Stiffening members 612 are attached to the outer surfaces of proximal end portion (such as by adhesive bonding thereto and/or mechanical fixation) or embedded in proximal end portion of conduit 600 to maintain a smooth, continuous surface interiorly where the lumen 609 is formed, so as to provide a smooth, continuous surface along which an implant and/or tools can be delivered while reducing friction to the extent possible. Likewise, as noted above, coil 610 is embedded so that it does not form a part of the inner surface that defines the lumen 609.

Stiffening members 612 may be thin strips of polymer, such as polycarbonate, Nylon, ABS, PEBAX, polyethylene, or the like that, when installed as shown, increase the column strength of the proximal end portion of conduit 600 to resist buckling, as well as longitudinal stretching of the proximal end portion under longitudinal forces that would cause buckling or stretching in the same proximal end portion when unreinforced by members 612. Stiffening members 612 may flare out at the proximal end portions thereof overlying the funnel portion 602 of conduit 600 to provide even more rigidification of the funnel portion, not only longitudinally, but also circumferentially. Slots and/or notches 608,608n may be provided to run longitudinally along conduit 600 between the stiffening members 612 to facilitate splitting the proximal end portion open in a manner described previously. Note that in this example, tabs 606 extend longitudinally and proximally from the proximal ends of stiffening members 612.

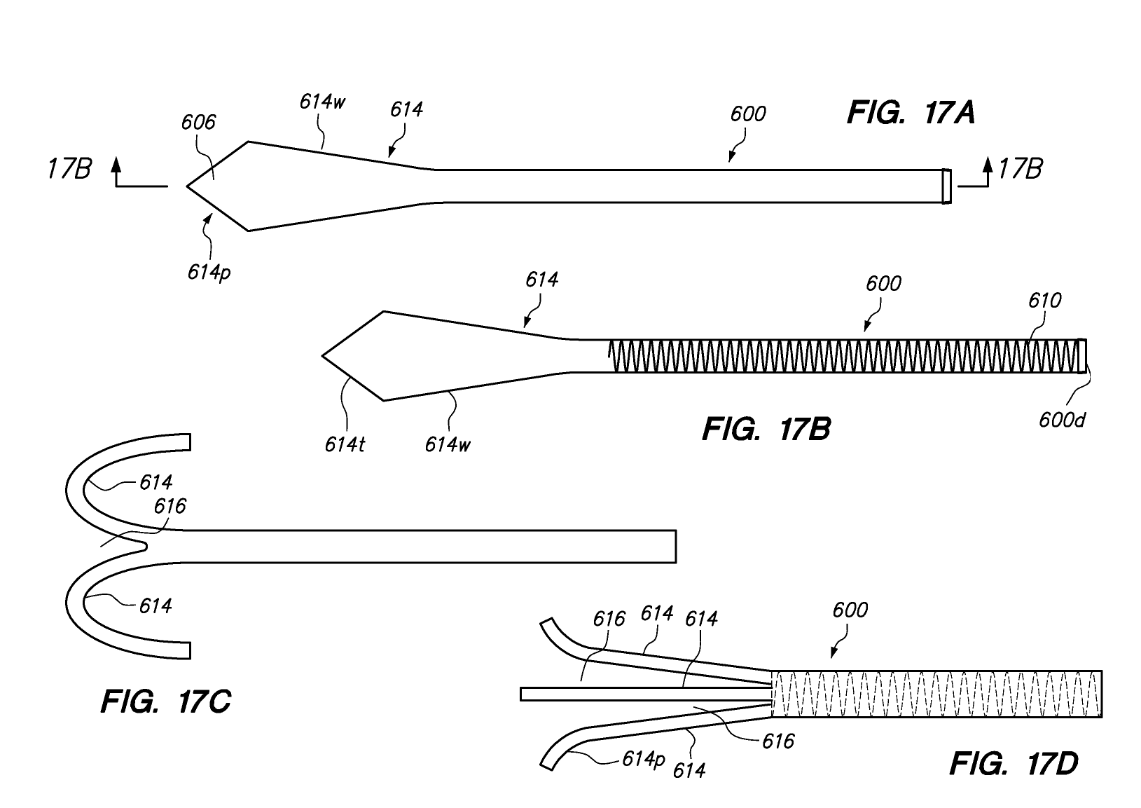

FIGS. 17A-17D illustrate alternative embodiments of conduit 600 according to the present invention. Like the embodiment of FIGS. 13A-13E, the main tube of the conduit 600 is flexible and is formed of an elastomer, such as silicone, and a coil 610, such as a stainless steel coil, Nitinol coil, or the like, is encapsulated in the elastomer along at least the distal end portion of the conduit 600. Also like the embodiment of FIGS. 13A-13E, the chamfered or tapered distal end 600d is not reinforced with the coil 610. At least a 4″ length of the conduit 600 extending proximally from the unreinforced distal end 600d is reinforced with coil 610. In other embodiments, a least a quarter or at least a third or at least half of the length of the conduit 600 extending proximally from the unreinforced distal end 600d is reinforced with coil 610. In the examples shown in FIGS. 17A-17D, coil 610 reinforces more than half of the entire length of the main body tube of conduit 600, and extends proximally from the unreinforced distal end 600d to a location distally adjacent the distal ends of “petals” 614 that open away from the opening into the distal portion of the conduit 600. The proximal end portion of the main tube of conduit 600 that is proximal of the proximal end of coil 610 is formed by petals 614 (two petals 614 in the embodiment shown in FIGS. 17A-17C, although more than two petals 614 may be employed to form the proximal end portion of conduit 600, e.g., see FIG. 17D). Petals 614 are thin, broad and elongated leaf-like structures that are flexible and are typically formed of the same material as the main tubular portion of conduit 600. These thin, flexible elongate members (petals) 614 are separated from one another along the lengths thereof by longitudinally extending spaces 616, and are connected/integral at their distal ends with the tubular portion of conduit 600. Petals 614 may flare or taper from their distal ends to form wider portions 614W. It is preferred to have the petals narrower at the distal ends to create more overall strength and rigidity on the proximal end, yet influence reliable bending on the distal end. With narrow distal ends, the petals bend at substantially the same locations every time and do so easier than would be the case if they were not narrowed.

Although petals 614 are not typically physically connected to one another along the lengths thereof, they can be held together by the hand of a user as a tool or implant is passed therethrough. Petals 614 can be subsequently bent/flexed apart as illustrated in FIG. 17C to reduce the overall length of conduit 600 when needed, or to increase the effective diameter of the annulus/lumen of the conduit at the proximal portion. The proximal-most portions of petals 614T may optionally be tapered to narrow back down to a narrow width proximal end to facilitate grasping by a user, whereby the proximal ends of the petals 614 function as tabs 606. Further optionally, the proximal ends 614p of petals 614 may be additionally or alternatively preshaped to flare radially outwardly as shown in FIG. 17D, to facilitate both grasping by the user and introduction of implants/tools into conduit 600.

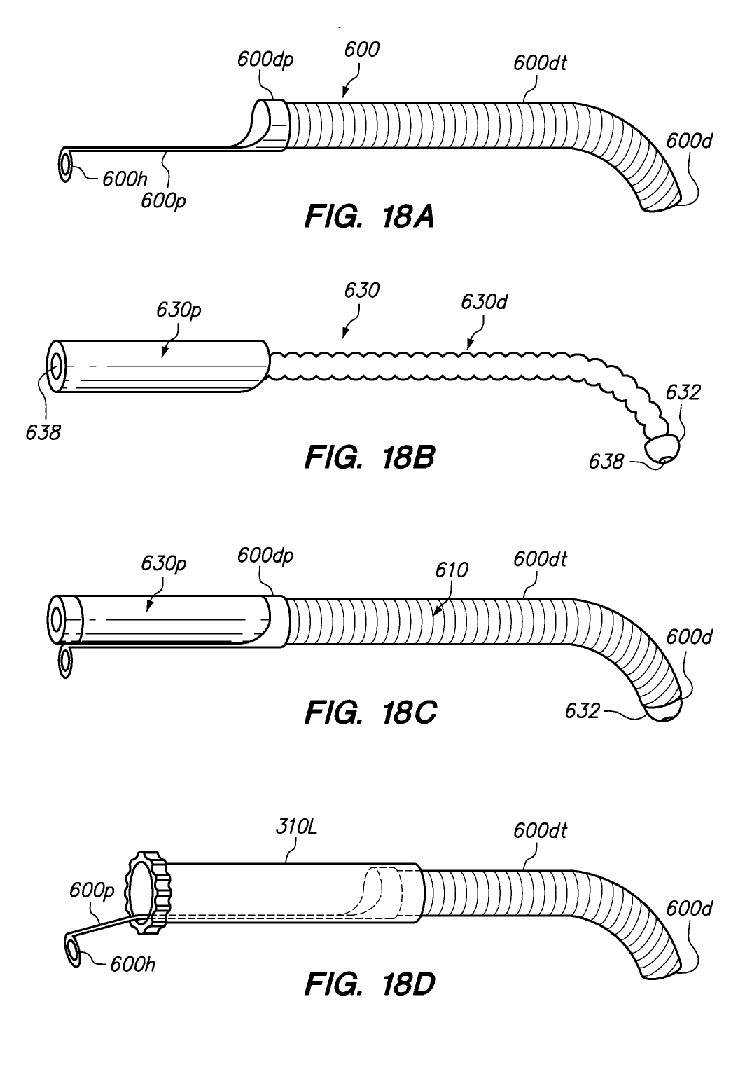

FIGS. 18A-18D illustrate alternative embodiments of conduit 600 and obturator 630 according to the present invention. Like the embodiment of FIGS. 13A-13E, the main tube of the conduit 600 is flexible and is formed of an elastomer, such as silicone, and a coil 610, such as a stainless steel coil, Nitinol coil, or the like, is encapsulated in the elastomer along at least the part of the distal end portion 600dt of the conduit 600. Also like the embodiment of FIGS. 13A-13E, the chamfered or tapered distal end 600d is not reinforced with the coil 610. At least a four inch length of the conduit 600 extending proximally from the unreinforced distal end 600d is reinforced with coil 610. In the example shown in FIGS. 18A, 18C and 18D, coil 610 reinforces substantially all of the tubular, distal end portion 600dt of conduit 600 except for the distal tip 600d, as noted, and a proximal end portion 600dp of the distal end portion 600dt.

The proximal end portion of 600p of conduit 600 in this embodiment is not tubular, but is rather an elongated member or “control stick” that extends proximally from proximal end portion 600dp of tubular distal end portion 600dt. Both proximal end portion 600dp and proximal end portion/control stick 600p may be formed of a more rigid material that that the elastomer used to make the tubular distal portion 600dt, to improve resistance to bucking during delivery of the conduit 600 over guide 530, as well as to improve control characteristics of the control stick 600p by reducing whip and other undesirable effects that would occur with a more flexible control stick. By making the proximal end portion 600p of the conduit 600 to be non-tubular and only a slender, rigid shaft or slick, this greatly reduces the amount of friction between the conduit 600 and large cannula 310L, so that if the operator needs to rotate or otherwise position the conduit 600 relative to the large cannula 310L, this action is easier to accomplish and is more accurately controlled by simply manipulating (rotating and/or pushing or pulling on) the proximal end of control stick 600p that extends proximally of the outer conduit 310L as illustrated in FIG. 18D.

Control stick 600p may include a handle 600h such as a ring or other structure located at a proximal end thereof and configured to facilitate grasping and manipulation by a user. There is also less of a pathway that an implant or tool needs to be inserted through conduit 600. For example, large cannula 310L can be formed of a more rigid material and can be made to reduce friction, such as by making it of polytetrafluoroethylene, expanded polytetrafluoroethylene or some other lubricious material, or at least coating the inner walls of the cannula 310L with the same. By providing the proximal opening of tubular distal portion with an angle in a direction from where proximal end 600dp meets control stick 600p to an opposite site of the proximal end 600dp, this also facilitates insertion of an implant into the tubular portion 600dt, when proximal end 600dp is contained within large cannula 310L as illustrated in FIG. 18D.

An embodiment of an obturator 630 configured for use with the embodiment of the conduit 600 shown in FIG. 18A is shown in FIG. 18B. The distal end portion 630d may be configured essentially the same as that described above with regard to FIG. 14C (or alternatively, FIG. 14A) for example. The proximal end portion is rigid and is configured to mate against the proximal end 600dp of distal end portion 600dt of conduit 600 when distal lip 600d is engaged with the distal tip 632 of obturator 630, as shown in FIG. 18C. Thus, when conduit 600 is assembled over obturator 630 as shown in FIG. 18C, obturator 630 helps prevent conduit from buckling, as well as from its walls collapsing inwardly, while still allowing distal portion 600dt to flex and bend as it is advanced over the guide 530 toward a surgical target location. The rigid proximal portion 630p of obturator 630 can be made of or coated with the same material that cannula 310L is made of or coated with, or made from or coated with a different material which is designed to have very low friction relative to the inner walls defining the annulus of cannula 310L. This facilitates advancement of conduit 600 by reducing friction at the proximal end.

Once conduit 600 has been delivered to or near the desired surgical target location, obturator 630 can be removed, as illustrated in FIG. 18D, while maintaining conduit 600 and cannula 310L in place. At this stage, cannula 600 can be further repositioned, tweaked, etc., if necessary, by manipulation of control stick 600p/handle 600h from a location outside of the patient. Implants and or tools can be inserted through cannula 310L and conduit 600 to deliver at least distal end portions thereof to the surgical target location distal of distal end 600d.

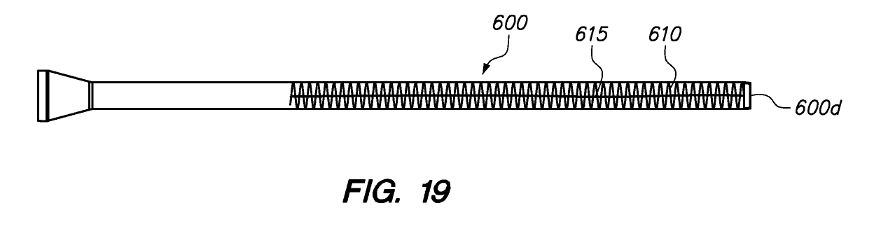

FIG. 19 illustrates an optional feature that may be provided with conduit 600 to resist stretching of the conduit 600 and/or to resist axial compression of the conduit 600. One or more resistive members 615 may be provided longitudinally along the main body of conduit 600. In the example shown in FIG. 19, one metallic wire extends along the entire length of coil 610 and is fixed (such as by soldering, welding, etc.) to at least two different coils of the coil 610 to prevent elongation thereof and also to fortify the resistance to buckling. Alternatively the one or more resistive members 615 may be provided along only a portion of the length of tube 600 and/or coil 610. Multiple resistive members 615 may be provided along various different longitudinal locations an/or various radial positions along the tube 600. Resistive member(s) need not connect to a coil 610, but can be embedded in or molded into a tube 600 that is not reinforced by coil 610. Further alternatively, resistive member(s) 615 may be made of flexible material, such as suture material or other polymer, in which case, it/they will prevent elongation of the tube, but will not necessarily fortify against buckling.

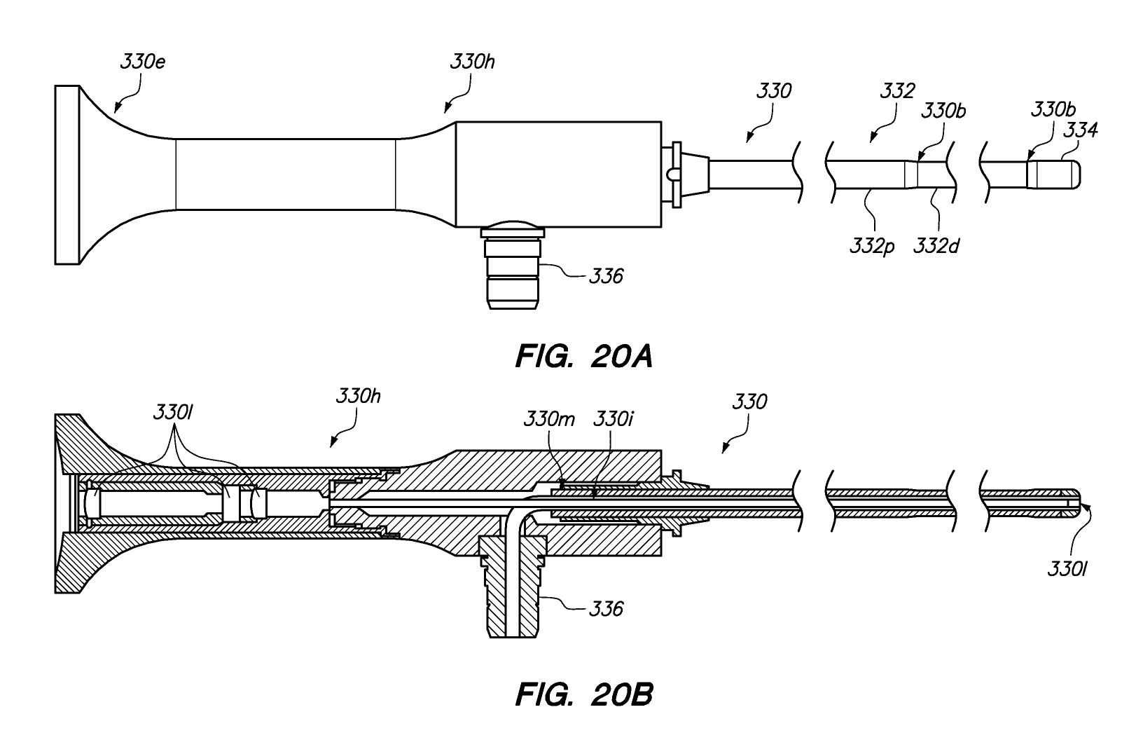

FIG. 20A is a partial view of an endoscope 330 that may be inserted into tube 534 of guide 530 and also may be inserted into conduit 600 or conduit 310L, in each instance, to provide visualization during performance of one or more steps of a procedure as described herein. FIG. 20B shows a longitudinal sectional view of FIG. 20A. The elongated shaft 332 is only partially shown in FIGS. 20A and 20B, so as to be able to show the views in a larger scale while still allowing them to fit on the page. The proximal portion 332p of shaft 332 is rigid, while the proximal portion 332d is flexible. The lengths of each portion 332p and 332d may vary. In one embodiment the length of rigid portion was about sixteen inches and the length of the distal portion 332d plus tip 334 was about twenty-seven inches.

Light post 336 is configured in the proximal handle portion 330h of the endoscope and, as noted previously, endoscope 330 can be inserted into conduit in a manner that light post 336 extends out of and slides along slot 608. An eye cup 330e is provided at the proximal end of the endoscope. Bevels 330b may be provided at the junctures of proximal with distal portions 332p, 332d and distal portion with distal tip 330d, 334. The maximum diameter of the elongated shaft 332 (including tip 334) in one embodiment is less than or equal to about five millimeters. In the same embodiment, the working length of the elongated shaft 332 (including tip 334) is about 42 inches to about 44 inches. The flexibility of distal flexible portion allows the guide 530 to bend, and therefore allows the endoscope 330 to be located in the guide 530 even when the guide is being inserted into the patient as it does not restrict the ability of the guide 530 to be steered or to bend, and it provides imaging to the surgeon so that the surgeon can see where the guide is being driven too. Additionally, the rigid portion 332p provides some stiffening support to the guide 530 to facilitate pushing the tube 530 into the patient.

Illumination fibers 330m extend through the main lumen of endoscope 330 and are connectable at a proximal end thereof to a light source (not shown) via light post 36 to deliver light out the distal tip 334 of endoscope 330. Lenses 330L are provided in the main lumen at the location of the distal tip 334 and proximal portion of the handle 330h to provide an image of the light reflected off of the environment as the illumination light exits the tip 334, reflects off objects and is reflected back into tip 334. Imaging fiber(s) connect the distal lens 330L with the proximal lens 330L arrangement in the handle 330h. A camera (not shown) may be connected to the endoscope for providing the ability to display images on a computer screen, provide image prints, etc.

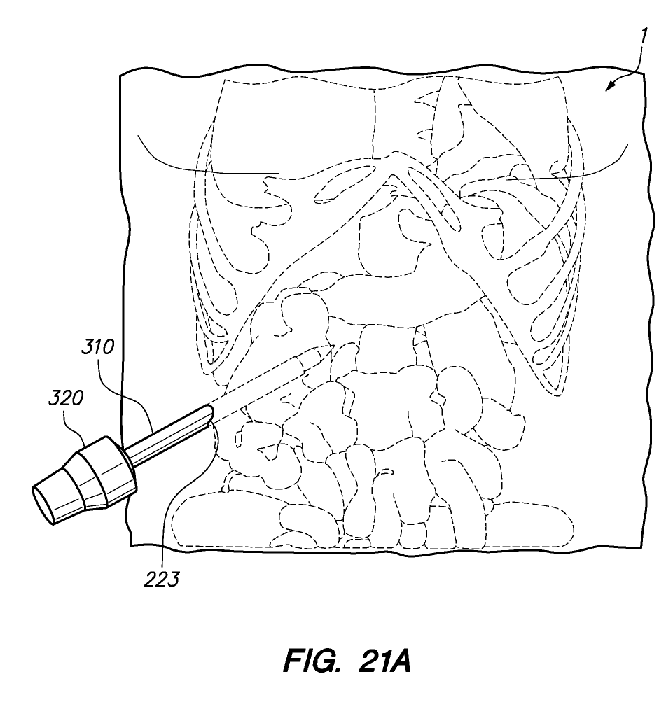

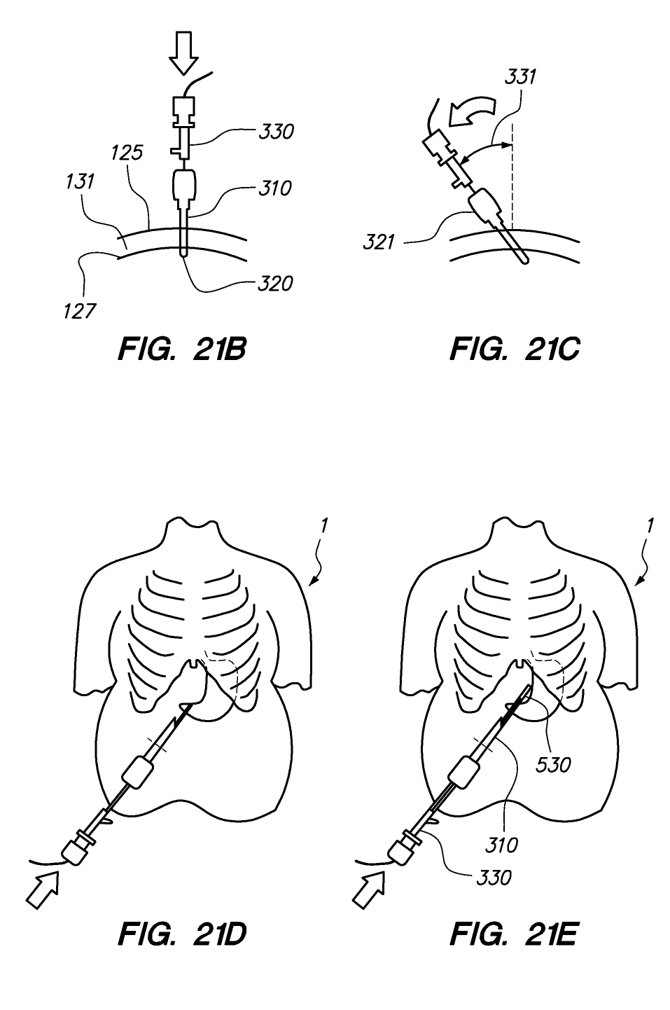

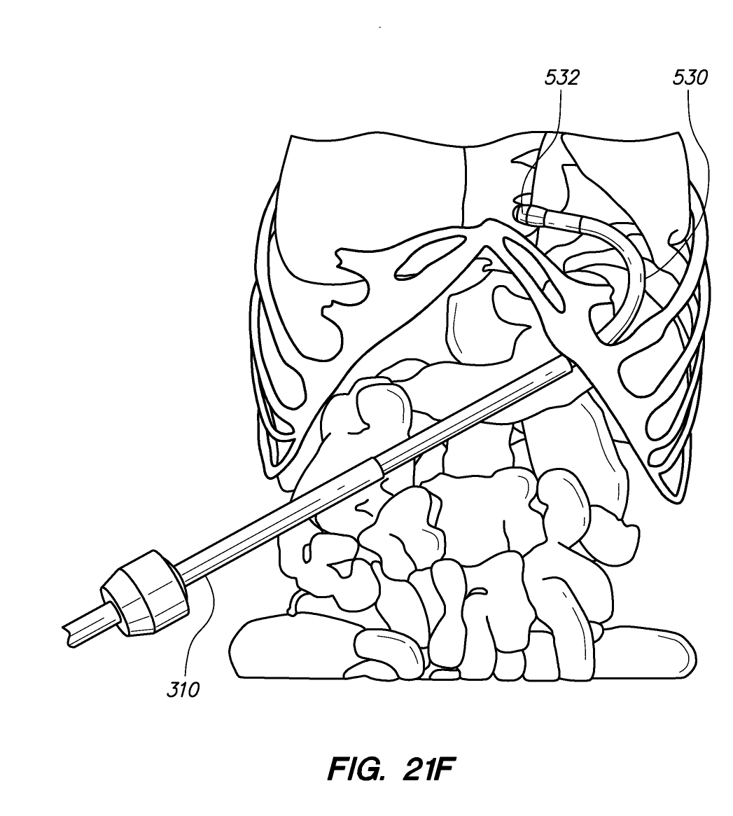

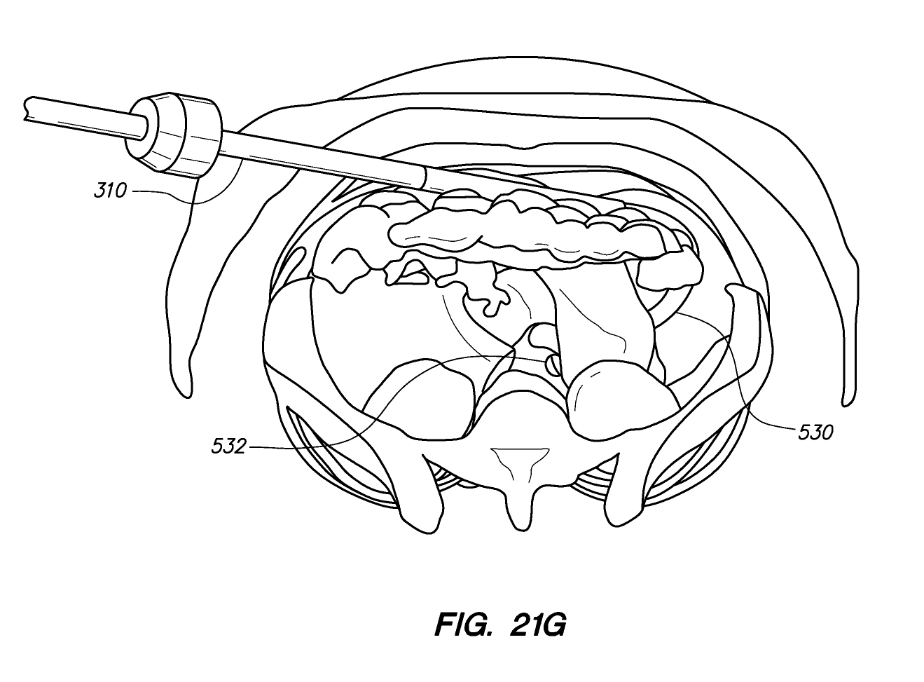

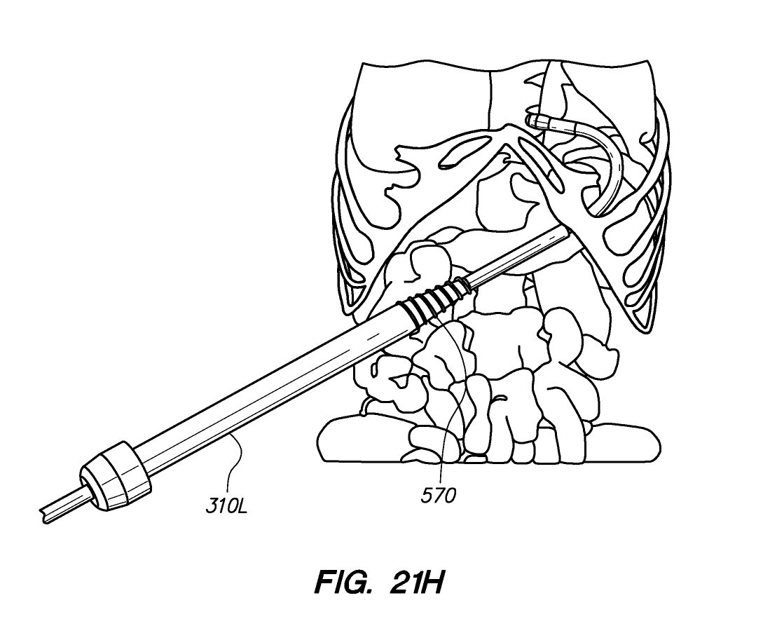

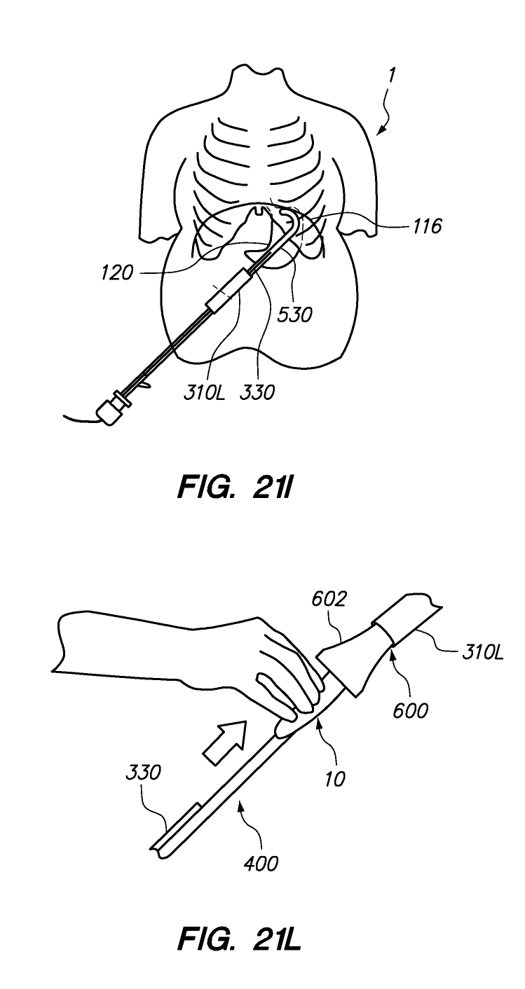

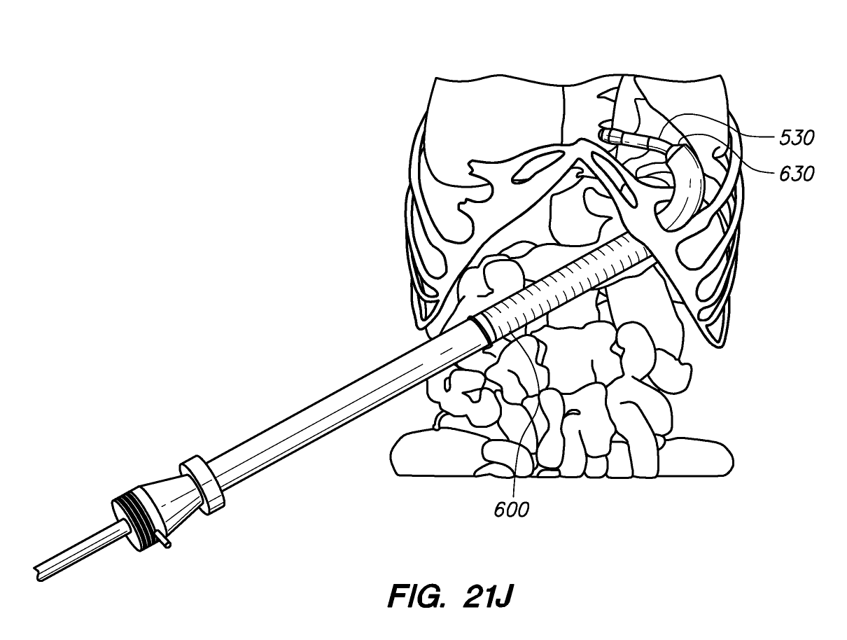

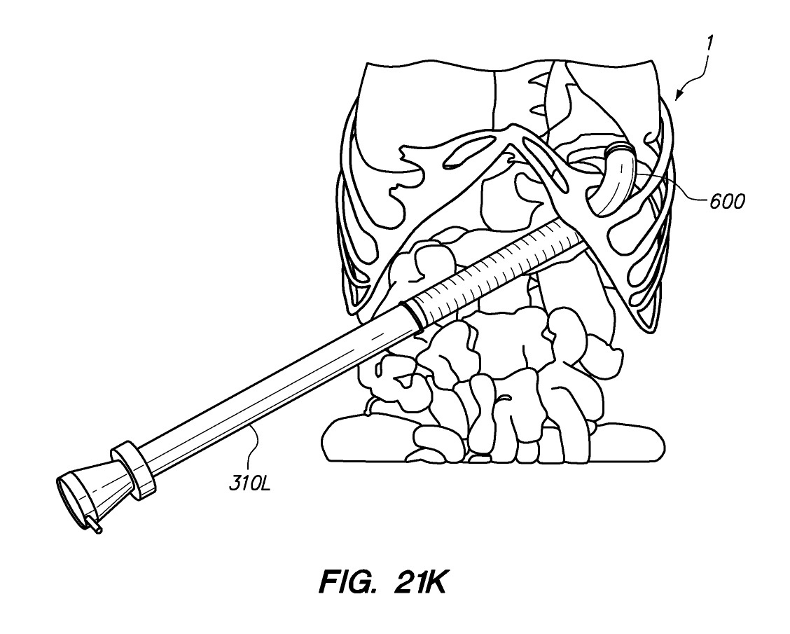

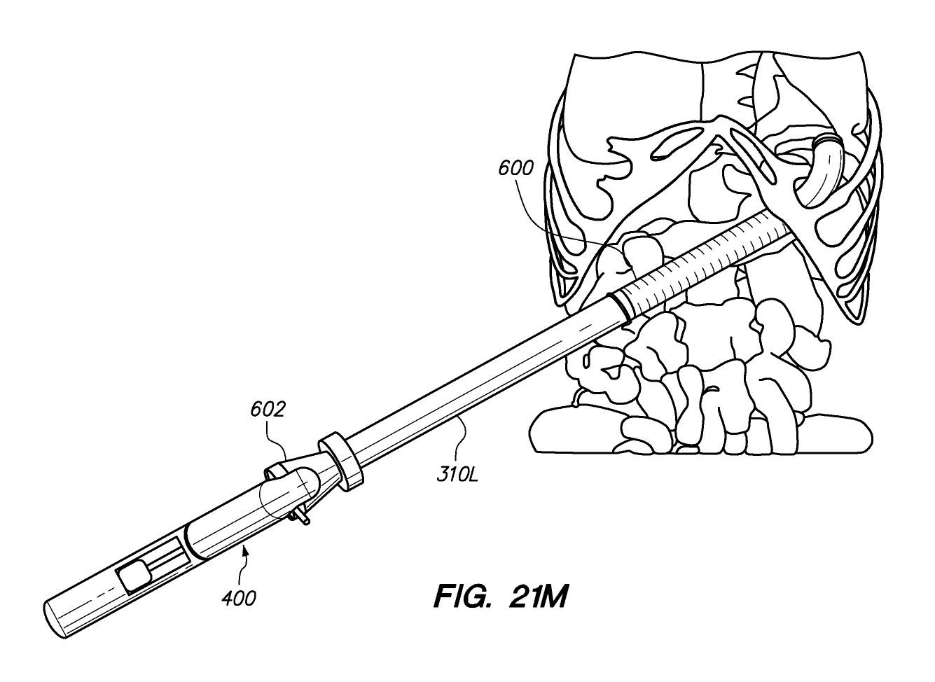

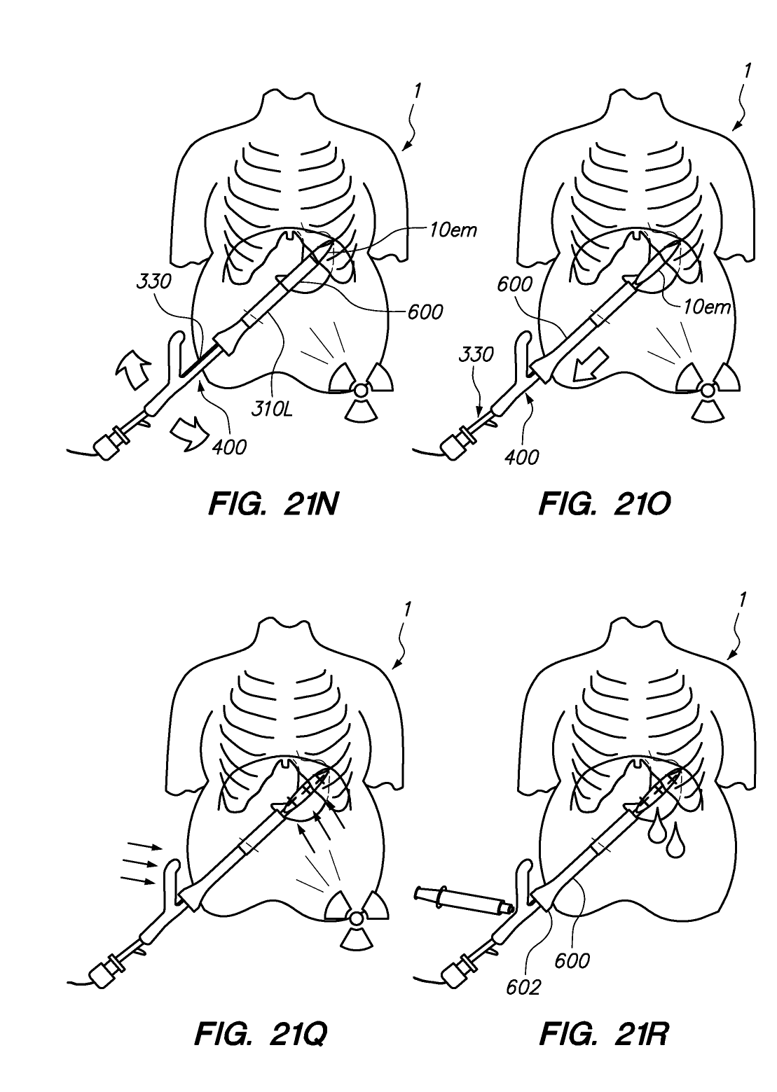

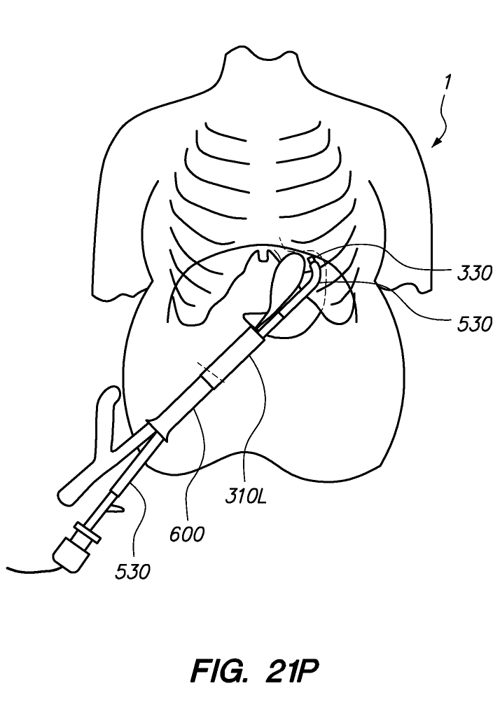

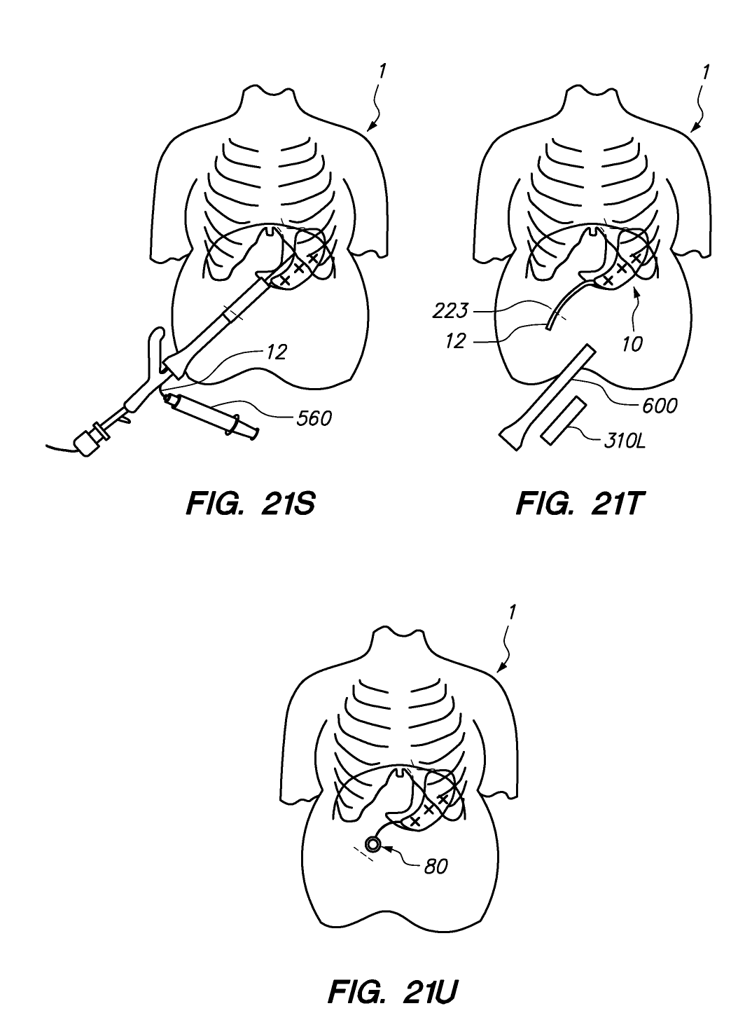

FIGS. 21A-21U illustrate an example and variations thereof of a procedure for percutaneously implanting an extra-gastric, paragastric device 10 according to the present invention. As already previously noted, the guide 530, obturator 630, conduit 600, introducer 310L, dilator 570 and endoscope 330 are not limited to the type of procedure described with regard to FIGS. 21A-21U, but this procedure is described in detail to facilitate a detailed understanding of the use of these devices, whether for the particular procedure described, or for other procedures in the body of a patient. After preparing the patient 1 for surgery, an incision 223 is made and a trocar/cannula 320/310 (e.g., a standard 15 cm length trocar/cannula) and 10 mm endoscope (shaft has 10 mm outside diameter) 330 are inserted into the incision and advanced under visualization by endoscope 330 (see FIGS. 21B-21C). Optionally, a small amount of insufflations gas may be inputted to help place the trocar/cannula in the desired layer(s) of tissues. In this embodiment, incision 223 is made at a predetermined distance inferior of the xyphoid process and a predetermined distance to the right of midline of the patient 1, see FIG. 21A. For example, the distance below the xyphoid process may be about 15 cm and the distance to the right of midline may be about 6 cm, although these distances may vary. Initially, the trocar 320, cannula 310 and endoscope 330 are inserted into incision 223 at a substantially perpendicular orientation to the surface of the skin 125, as schematically illustrated in FIG. 21B. Once the sharpened tip of the trocar 320 has passed through the fascia 127f/abdominal muscle 127 and it and the distal tip of the cannula 310 have entered the abdominal cavity, the trajectory of the cannula 310, trocar 320 and endoscope 330 is flattened relative to the skin of the patient surrounding the incision 223, as schematically illustrated in FIG. 21C (and which orientation is also illustrated at FIG. 21A) to form an angle 331 relative to the original, perpendicular orientation of greater than about 60 degrees, typically greater than about 80 degrees, and, in some embodiments, 90 degrees or more. A delivery tract is thus formed as described above, and endoscope 330 is inserted distally to view along the tract up to the location of the intra-abdominal fat or possibly as far as the location of the stomach 120, as shown in FIG. 21 D. The trocar 320 and endoscope 330 are then removed. Guide 530 is next inserted into the tract and a smaller endoscope 330 (e.g., endoscope shaft having about 2 mm to about 5 mm outside diameter, which may be the endoscope 330 described above with regard to FIGS. 20A-20B, for example) is introduced into guide 530. Guide 530 and endoscope 330 are manipulated in a manner as described above to establish a pathway into a space between the fascia and the bowel, see FIG. 21E. This procedures allows users to use a small amount of CO₂, if desired, to help get the guide 530 past the falciform and through the correct layers of tissues. If a flexible endoscope 330 is used, or an endoscope 330 that is flexible at least along a distal portion of the endoscope shaft 332d, alternatively to the rigid endoscope 330 shown in FIG. 21D, then viewing can be extended up to and along the diaphragm 116, for example, as illustrated in FIG. 21F. FIG. 21G illustrates a sectional view, where it can be readily observed that the tip 532 of the guide 530 also traverses around the stomach and dives down into the abdominal cavity as it is guided by the curvature of the diaphragm.

The cannula 310 and smaller endoscope 330 are then removed while leaving the guide 530 in place. Dilator 570 is next screwed and/or pushed through opening 223 and the opening through the fascia to enlarge the opening through the fascia/abdominal muscle 127f/127, to install a large cannula 310L, see FIG. 21H. During this procedure, a dilator 570 that includes at least one endoscope port 570p and which has a transparent tube 570n may be alternatively used with an introducer 310L that has a transparent tube 310t and an endoscope 330 can be inserted like shown in FIG. 11C to provide a view for the surgeon to observe the dilation procedure as it is performed. Once large cannula 310L is installed through the enlarged opening in the fascia, dilator 570 is removed, the smaller endoscope 330 can be reinserted into guide 530, which now extends through the large cannula 310L, see FIG. 21I. Guide 530 is stiffened by endoscope 330 (when a rigid endoscope 330 is used, or an endoscope like in FIGS. 20A-20B, where at least a proximal portion 332p of the endoscope shaft is rigid) which acts as a stylet as the guide 530 and endoscope 330 are advanced to establish the delivery tract to the diaphragm, between the fascia and bowel, and to view the diaphragm 116. Guide 530 is then advanced further, such that the distal portion does not contain endoscope 330 (when a rigid endoscope is used) so that it is floppy and follows around the curvature of the diaphragm 116 as illustrated in FIG. 21I. When endoscope 330 is flexible, or has at least a flexible distal portion 332p of the shaft, it can be inserted into the distal portion of guide 530 and follow with it along the bending trajectory that follows along the curvature of the diaphragm. Endoscope 330 can be used to view the advancement of guide 530 as well as to check the areas surrounding the delivery tract leading to the diaphragm 116. As noted, a flexible endoscope 330 may alternatively be inserted so that it remains within the flexible distal end portion of guide 530 as it is advanced along the diaphragm, so that this travel can be visualized via endoscope 330. T is alternative is described in further detail below. Otherwise, when a rigid endoscope 330 is used, the flexible distal end portion of guide 530 can be tracked under fluoroscopy when one or more radiopaque markers are included on the flexible distal end portion of guide 530.

Endoscope 330 is next removed, and a conduit 600 and obturator 630 are inserted into the abdominal cavity, being guided over guide 530 as illustrated in FIG. 21J. Once the distal end of the conduit 600 has been advanced to a position adjacent the diaphragm 116 (when a rigid conduit 600 is used), or adjacent to the target implantation site after following around the curvature of the diaphragm 116 when a flexible conduit 600 as used as illustrated in FIG. 21J, guide 530 and obturator 630 are removed, leaving conduit 600 in position for guiding delivery of device 10, as illustrated in FIG. 21K. Alternative to use of an rigid conduit 600, a flexible conduit 600 and flexible obturator are preferably used, as shown in FIGS. 21J-21K. At least the distal end portion of each of conduit 600 and obturator 630 is flexible. The flexible distal end portions are configured to follow the flexible distal end portion of the guide 530 so that the distal end portion of the conduit can be delivered along the diaphragm 116 close to or flush with (or even extending slightly distally of) the distal end of guide 530, as described in further detail below.

A delivery and attachment tool 400 having received the smaller endoscope (e.g., 5 mm endoscope or 2.7 mm endoscope) 330 therein and having had device 10 mounted thereon, where device 10 is in a compact configuration, is next operated to insert the device 10 and tool 400 into the conduit 600 as illustrated in FIG. 21L.

At FIG. 21M device 10 is advanced into the abdominal cavity by advancing tool 400 relative to conduit 600 until the distal end portion 10em of the device 10 is located at or extends distally of the distal end of conduit 600, as shown in FIG. 21N. This location of the device 10 can be determined by one or more of monitoring the amount of the tool 400 that remains proximal of the proximal end of conduit 600, as the length of the tool 400 with device 10 mounted thereon relative to the length of conduit 600 may be known or predetermined; visual monitoring via endoscope 330; and/or visual monitoring by fluoroscopy. At this time, the position of the portion 10em of device 10 relative to the anatomy can also be adjusted, if needed, using tool 400 and/or conduit 600 to adjust the position of the device 10 monitoring movements of the device 10 using fluoroscopic visualization. Further repositioning of the device 10 can be performed at this time as well, using tool 400 under fluoroscopic guidance.

Conduit 600 is next retracted relative to tool 400 to fully expose the compacted expandable member 10em of device 10 as shown in FIG. 21O. This action can also be visually monitored under fluoroscopy. FIG. 21P shows an alternative embodiment, where tool 400 does not receive endoscope 330, and where, after expandable member 10em has been exposed out of the distal end of conduit 600, guide 530 having received endoscope 330 is inserted through conduit 600 to provide visualization of the device 10em at the target site. Endoscopic visualization via endoscope 330 is used to confirm that the attachment location is clear of bowel, e.g., that the tool 400 and portion of the device 10 to be attached are positioned so that a clear pathway to the attachment site exists, such that no bowel, excessive fat or other obstruction exists between the attachment tab and the attachment location, such as the abdominal wall, costal cartilage, or other internal body structure to which device 10 is to be attached. When a clear pathway has been confirmed, the operator manipulates tool 400 via the handles to leverage the attachment portion of device 10 against the attachment site so that the portion contacts the attachment site where it is to be anchored. The operator then actuates tool 400 to fire anchor divers and deploy anchors through the attachment portion of device 10 and attachment structure (e.g., fascia and/or other internal body structure), the drivers are retracted to leave the anchors in place, and sutures are cinched up against the attachment portion and attachment structure to anchor device 10 in place, with the attachment portion of device 10 and internal body structure attachment site sandwiched between the anchors or other fixation mechanism via sutures, as schematically represented in FIG. 21Q.

Next, a local anesthetic, such as Marcaine, or the like can be delivered to the target implantation site through a lumen in tool 400 as illustrated in FIG. 21R, such as through a lumen extending through tool 400 adjacent the lumen that endoscope 330 is received in. At FIG. 21S a source of pressurized fluid 560 is next connected to fill tube 12 and fillable member 10em is at least partially filled with the fluid.

Tool 400 is decoupled from device 10 and then removed from conduit 600. Conduit 600 and large cannula 310L may also be removed from the patient at this time, as schematically illustrated in FIG. 21T. Fill tube 12, extends proximally out of opening 223, as illustrated in FIG. 21T.

At FIG. 21U, fill tube 12 is cut to the appropriate length to join adjustment member 80 thereto and to reduce any excessive length of fill tube 12 that might otherwise exist. After securing adjustment member 80 to the fascia 127f/abdominal wall 127 to both anchor it as well as to close the opening through the fascia 127f, any adjustment of the volume of expandable member can be performed as needed, and then the patient can be closed, including closing of opening 223 to complete the procedure. As in other embodiments, adjustment member 80 can be installed attached to the abdominal wall 127/fascia 127f at a location other than the opening 223. In such cases, opening 223 is closed around the fill tube 12 extending therefrom, and the adjustment member is attached to the fascia 127f and or abdominal muscle 127 at another location, so that attachment member 80 does not need to perform the closure function for closing the opening 223. Further details of this and other procedures that can be performed with the devices of the present invention are described in Application Ser. No. 61/130,244, co-pending application Ser. No. (application Ser. No. not yet assigned, Attorney's Docket No. EXPL-008), and application Ser. No. (application Ser. No. not yet assigned, Attorney's Docket No. EXPL-012), each of which were incorporated herein above, in their entireties, by reference thereto.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A guide device configured to be inserted through an opening in the skin to form a delivery tract to a target location within the body of a patient, said guide device comprising:
an elongated tube, at least a portion of which is transparent;
a transparent distal tip closing a distal end of a main lumen of said elongated tube and having a blunt exterior surface;
wherein a proximal end of said main lumen of said elongated tube is open and communicates with said main lumen, said main lumen extending through said elongated tube; and
wherein a distal portion of said elongated tube is flexible.

2. The device of claim 11, wherein said distal tip comprises a lens.

3. The device of claim 1, wherein said lens comprises an exterior surface having a first curvature and an interior surface having a second curvature different from said first curvature.

4. The device of claim 3, wherein said interior surface is duckbill-shaped.

5. The device of claim 3, wherein said exterior surface is blunt and said tip is rigid.

6. The device of claim 1, further comprising a rigid tube configured to be slidably received within said elongated tube, a distal portion of said elongated tube being flexible, at least a portion of said rigid tube being slidable into said distal portion to temporarily rigidify said distal portion.

7. The device of claim 1, wherein said rigid tube comprises a central lumen configured to slidaby receive an endoscope therethrough.

8. The device of claim 6, further comprising an actuator extending externally of said elongated tube, said actuator being actuatable to slide said rigid tube relative to said elongated tube.

9. The device of claim 8, wherein said actuator is movable between an active configuration and an inactive configuration, said actuator being actuatable to slide said rigid tube relative to said elongated tube when in said active configuration, said actuator extending substantially flush with said elongated tube when in said inactive configuration.

10. The device of claim 1, wherein at least a distal portion of said elongated tube is transparent.

11. The device of claim 1, wherein a distal end portion of said elongated tube is pre-bent along a curvature, relative to a longitudinal axis of a straight main portion of said elongated tube, said pre-bent portion configured for steering said device.

12. The device of claim 9, further comprising an extension tube, a distal end portion of said extension tube being configured to be slide over a proximal end portion of said elongated tube and said actuator in said inactive configuration.

13. An assembly for providing access to a target location within the body of a patient, said assembly comprising:
a guide member comprising an elongated tube, at least a portion of which is transparent and at least a portion of which is rigid, or rigidified by receiving a stiffening member therein, and a transparent distal tip closing a distal end of a main lumen of said elongated tube and having a blunt exterior surface, a proximal end of said main lumen of said elongated tube being open;
a conduit having a length sufficient to extend from said target location, at a distal end of said conduit through an opening in the body such that a proximal end of said conduit extends out of the body, at least a portion of said conduit being flexible, said conduit configured to allow at least one device or tool to be delivered therethrough; and
an obturator having a length greater than an length of said conduit, said obturator being flexible and configured to be inserted through said conduit, a distal end portion of said obturator configured to extend distally of a distal end of said conduit and seat against said distal end, said obturator further comprising a central obturator lumen, said central obturator lumen configured to closely follow over said guide member, so that when inserted into said conduit said obturator and said conduit form an obturator and conduit assembly and when said obturator and conduit assembly is passed into the body over said guide member, said distal end portion of said obturator, together with said guide member substantially prevents bodily fluids and tissues from entering said conduit.

14. The assembly of claim 13, further comprising:
a dilator having a tapered distal end portion and a tubular proximal end portion, said dilator having a central dilator lumen, said central dilator lumen configured to closely follow over said guide member.

15. The assembly of claim 14, further comprising:
a blunt edged threadform spiraling around said tapered distal end portion of the dilator.

16. The assembly of claim 14, further comprising:
a cannula configured to be slid over said tubular proximal end portion of said dilator, to follow the dilator as it is drawn in through an opening as the Bator is torqued, said cannula having a central cannula lumen having a diameter greater than an outside diameter of said conduit.

17. The assembly of claim 16, wherein said cannula comprises threading on a distal end portion thereof.
